(19) United States
(12) Patent Application Publication
(10) Pub. No.: US 2026/0001892 A1
(43) Pub. Date: Jan. 1, 2026

(54) COMPOUND FOR REGULATING AND CONTROLLING ACTIVITY OF 15-PGDH AND PREPARATION METHOD THEREFOR

(71) Applicant: SCINNOHUB PHARMACEUTICAL CO., LTD., Chengdu, Sichuan Province (CN)

(72) Inventors: Sen JI, Chengdu, Sichuan Province (CN); Hao WANG, Chengdu, Sichuan Province (CN); Wenqing ZHANG, Chengdu, Sichuan Province (CN); Xiao WANG, Chengdu, Sichuan Province (CN); Nan LI, Chengdu, Sichuan Province (CN); Jing WANG, Chengdu, Sichuan Province (CN); Yuanqing TANG, Chengdu, Sichuan Province (CN); Xiaodong ZHANG, Chengdu, Sichuan Province (CN); Jun TANG, Chengdu, Sichuan Province (CN)

(21) Appl. No.: 18/992,307

(22) PCT Filed: Jul. 21, 2023
(86) PCT No.: PCT/CN2023/108559

§ 371 Date: Jan. 8, 2025

(30) Foreign Application Priority Data
Jul. 22, 2022 (CN) 202210871173.5

(51) Int. Cl. C07D 495/04; A61K 31/4985; A61K 31/506; A61K 31/5377; C07D 519/00

(52) U.S. Cl. CPC C07D 495/04; A61K 31/4985; A61K 31/506; A61K 31/5377; C07D 519/00

(57) ABSTRACT

Provided are a compound of formula (I) having an effect of regulating and controlling the activity of 15-PGDH, a stereoisomer, a tautomer or a form of a mixture thereof, or a pharmaceutically acceptable salt thereof, or a solvate thereof, or a prodrug thereof, a pharmaceutical composition comprising same, and a preparation method therefor and the use thereof as a 15-PGDH inhibitor.

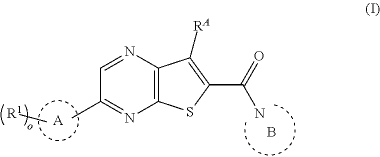

### TECHNICAL FIELD

The present application relates to a compound for regulating the activity of 15-PGDH and a preparation method thereof, specifically relates to a compound that can be used as a medicament for regulating the activity of 15-PGDH, and a pharmaceutically acceptable salt thereof, a composition comprising the compound or the salt thereof, and use thereof in the preparation of a medicament, belonging to the field of pharmaceutical chemistry.

### BACKGROUND

15-hydroxyprostaglandin dehydrogenase (15-PGDH) belongs to an evolutionarily conserved superfamily of short-chain dehydrogenases/reductases (SDRs), and has been designated as SDR36C1 according to the recently approved nomenclature for human enzymes. Based on current research results, the majority of the in vivo activity can be attributed to HPGD gene-encoded type I 15-PGDH. 15-PGDH plays an important role in the inactivation of active prostaglandins (PGD2, PGE1, PGE2, PGF2α, PGI2, etc.), hydroxyeicosatetraenoic acids (HETEs), and inflammation-resolving lipid mediators (RvD1, RvD2, RvE1, MaR1, LXA4, etc.) (hereinafter referred to generally as 15-PGDH substrates) (e.g., by catalyzing an oxidation reaction of hydroxy at position 15 of PGF2α into 15-keto-PGF2α). These prostaglandins (PGD2, PGE1, PGE2, PGF2α, PGI2, etc.), hydroxyeicosatetraenoic acids, and inflammation-resolving lipid mediators (RvD1, RvD2, RvE1, MaR1, LXA4, etc.) exert their functions through specific receptors on target cells. Among others, prostaglandins, such as PGE1, PGE2, PGF2α, PGI2, etc., are often used to assess the activity of 15-PGDH. For example, the activity of PGDH is assessed by measuring ketone metabolites of hydroxy at position 15 of PGF2α (Journal of Clinical Endocrinology and Metabolism, Vol 84, No. 1, 291-299).

Receptors of 15-PGDH substrates are widely and differentially distributed in vivo, and the diversity of expression distributions, receptor types, and signaling together create a diversity of effects in vivo. For example, PGE1 acts on blood vessels and platelets to exhibit an effect of increasing blood flow by vasodilatory effects and inhibition of platelet aggregation, and is therefore commonly used for treating diseases such as chronic arterial occlusion (thromboangiitis obliterans (TAO) or occlusive arteriosclerosis obliterans (ASO)), skin ulcers; PGF2α has uterine constriction and intraocular pressure lowering effects, and derivatives thereof have been used as therapeutic agents for glaucoma; and PGD2 inhibits inflammation by enhancing the barrier function of the pulmonary blood vessels. In addition, PGE2 has vasodilatory effects, and also has several functions including a variety of effects involving blood pressure, pain, bone formation and cell growth, stem cell differentiation, and anti-fibrotic and anti-inflammatory effects, etc. PGI2 has an inhibitory effect on platelet activation and a relaxing effect on vascular smooth muscle, and its derivatives are used as therapeutic agents for chronic arterial occlusion and primary pulmonary hypertension. Inflammation-resolving lipid mediators (RvD1, RvD2, RvE1, MaR1, LXA4, etc.) inhibit migration/activation of neutrophils and accelerate apoptosis of neutrophils. In addition, it is indispensable in the process of increasing the phagocytic activity of macrophages to effectively remove apoptotic neutrophils/tissue debris remaining at the site of inflammation. These functions can promote inflammation and maintain homeostasis within the organism. These inflammation-resolving lipid mediators have been reported to show medicinal efficacy in various types of pathology models (e.g., mouse pulmonary inflammation model, colitis model, and liver injury model).

Recent studies indicate that 15-PGDH inhibitors and 15-PGDH agonists may have therapeutic values. A recent study indicates that the expression of 15-PGDH in protection against thrombin-mediated cell death is increased. It is well known that the 15-PGDH causes the inactivation of prostaglandin E2 (PGE2), and the prostaglandin E2 is a downstream product of COX-2 metabolism. Studies have shown that PGE2 is beneficial in a variety of biological processes, such as maintaining hair density, promoting skin wound healing and bone formation.

15-PGDH as an important enzyme in the inactivation of 15-PGDH substrates involves a wide range of in vivo roles, and 15-PGDH inhibitors may be used to prevent or treat diseases associated with 15-PGDH and/or 15-PGDH substrates and/or when there is a need to increase the level of 15-PGDH substrates in the body of a subject.

As mentioned above, some substrates of 15-PGDH have anti-fibrotic, anti-inflammatory, blood flow improvement, growth-promoting, stem cell increase-promoting, smooth muscle contraction/relaxation-promoting, immunosuppression and bone metabolism-affecting effects, etc. Thus, 15-PGDH inhibitors may effectively treat or prevent fibrosis (e.g., pulmonary fibrosis (idiopathic pulmonary fibrosis, etc.), hepatic fibrosis, renal fibrosis, myocardial fibrosis, scleroderma, and myelofibrosis), inflammatory diseases (e.g., chronic obstructive pulmonary disease (COPD), acute lung injuries, sepsis, lung disease and asthma, inflammatory bowel disease (e.g., ulcerative colitis and Crohn's disease), peptic ulcers (e.g., NSAID-induced ulcers), autoinflammatory diseases (e.g., Behçet's disease), vasculitis syndromes, acute liver injury, acute kidney injury, nonalcoholic steatohepatitis (NASH), atopic dermatitis, psoriasis, interstitial cystitis, prostatitis syndrome (e.g., chronic prostatitis/chronic pelvic pain syndrome)), and cardiovascular disease (e.g., pulmonary arterial hypertension, angina pectoris, myocardial infarction, heart failure, ischemic heart disease, chronic kidney disease, renal failure, cerebral apoplexy, and peripheral circulatory disorders), trauma (e.g., diabetic ulcers, burns, pressure ulcers, acute mucosal injuries (including Stevens-Johnson syndrome; and mucosal injuries associated with alkylating agents, inhibitors of DNA synthesis, inhibitors of DNA gyrase, or antimetabolites and other anticancer chemotherapeutic agents, mucosal injuries associated with cellular or humoral immunotherapy or radiation therapy, or mucosal injuries associated with graft-versus-host disease, such as mucositis or stomatitis)), autoimmune diseases (e.g., multiple sclerosis or rheumatoid arthritis), graft-versus-host disease (GVHD), hair growth disorder, osteoporosis, ear diseases (e.g., hearing loss, tinnitus, vertigo, and dysequilibrium), eye diseases (e.g., glaucoma and dry eye), diabetes mellitus, underactive bladder, neutropenia, neurological diseases caused by transplantation of stem cells, bone marrows or organs (e.g., psychoneurosis, neuropathies, neurotoxic diseases, neuropathic pain, and neurodegenerative diseases), and muscle regenerative diseases (e.g., muscular dystrophy, myodystrophy, and muscle injuries); in addition, 15-PGDH inhibitors may also be used to promote cervical ripening.

Compounds and pharmaceutically acceptable salts thereof and the like provided in the present application further meet the requirement of small molecules for inhibiting the activity of 15-PGDH.

### SUMMARY OF THE INVENTION

One aspect of the present application provides a compound represented by formula (I), a stereoisomer, tautomer or mixture form thereof, or a pharmaceutically acceptable salt thereof, or a solvate (e.g., a hydrate) thereof, or a prodrug thereof:

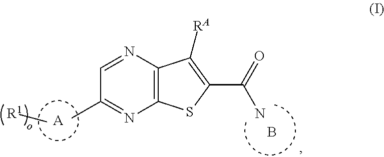

ring A is selected from an aromatic ring, an aromatic heterocycle, an unsaturated aliphatic heterocycle, a fused ring consisted of an aromatic ring and an unsaturated aliphatic heterocycle, and a fused ring consisted of an aromatic heterocycle and an unsaturated aliphatic heterocycle;
ring B is a 3-12 membered saturated aliphatic heterocycle;
R^{A} is selected from hydrogen, deuterium, tritium, hydroxy, halogen, cyano, C₁-C₆ alkyl, C₁-C₆ alkoxy, C₁-C₆ haloalkyl, and 3-8 membered cycloalkyl;
o is selected from 0, 1, 2, and 3;
R¹ is each independently selected from deuterium, tritium, nitro, hydroxy, mercapto, halogen, cyano, =O, imino, an amine group, an ester group, an aldehyde group, carboxyl, amido, C₁-C₆ alkyl, C₁-C₆ halogenated alkyl, C₁-C₆ alkoxy, C₁-C₆ halogenated alkoxy, 3-8 membered cycloalkyl, and 3-8 membered saturated aliphatic heterocyclyl;
wherein the aromatic heterocycle, the saturated aliphatic heterocycle, the unsaturated aliphatic heterocycle, the aliphatic heterocyclyl, and the fused ring each independently comprise 1-3 heteroatoms which are independently selected from N, O, and S, and ring B comprises at least 1 nitrogen atom;
the ring B and R¹ are optionally substituted by one or more independently selected from deuterium, tritium, nitro, hydroxy, —NH₂, mercapto, halogen, cyano, an ester group, carboxyl, amido, =O, ═NH, C₁-C₆ alkyl, C₁-C₆ alkoxy, 3-8 membered cycloalkyl, 6-10 membered aryl, 5-10 membered aliphatic heterocyclyl, and 5-10 membered heteroaryl;
further, the ring B is preferably a monocyclic ring or a bicyclic ring.

In some embodiments, the ring B is a 5-10 membered saturated aliphatic heterocycle comprising at least 1 nitrogen atom, e.g., a 5-8 membered or 5-7 membered saturated aliphatic heterocycle.

Further, in certain embodiments of the present application, the ring B is selected from

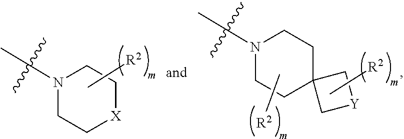

wherein X is selected from a covalent bond, O, S, NH, (CH₂)ₙ, and SO₂; Y is selected from a covalent bond, S, NH, (CH₂)ₙ, and SO₂;
m is selected from 0, 1, 2, and 3; R² is each independently selected from deuterium, tritium, nitro, hydroxy, mercapto, cyano, halogen, an amine group, an ester group, an aldehyde group, carboxyl, amido, C₁-C₆ alkyl, C₁-C₆ haloalkyl, C₁-C₆ alkoxy, 3-8 membered cycloalkyl, 6-10 membered aryl, and 5-10 membered heteroaryl; and n is selected from 0, 1, 2, and 3.

Further, in certain specific embodiments, the ring B is selected from

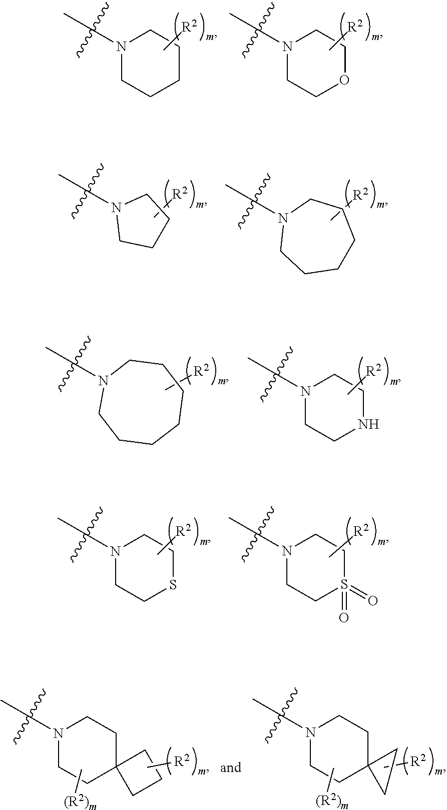

wherein m and R² are the same as defined above in the present application.

In some preferred embodiments, the ring B is selected from

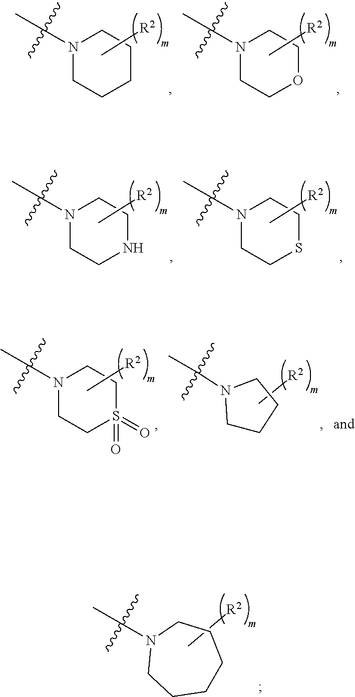

preferably

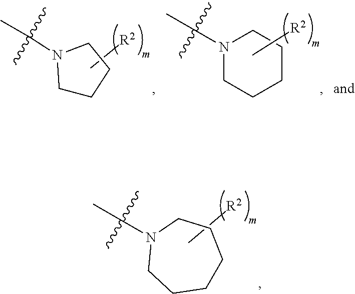

wherein m and R² are the same as defined above in the present application.

Further, in certain specific embodiments of the present application, the R² is each independently selected from deuterium, tritium, nitro, hydroxy, mercapto, cyano, fluoro, chloro, bromo, an amine group, an ester group, an aldehyde group, carboxyl, amido, methyl, ethyl, n-propyl, isopropyl, methoxy, ethoxy, n-propoxy, isopropoxy, trifluoromethyl, trifluoroethyl, trichloromethyl, trichloroethyl, cyclobutyl, cyclopropyl, phenyl, and pyridyl.

In some embodiments, the R² is each independently selected from deuterium, tritium, hydroxy, fluoro, chloro, bromo, an amine group, methyl, and ethyl; preferably deuterium and tritium.

In some embodiments, m is selected from 0, 1, and 2, e.g., 0 or 1, preferably 0.

In some embodiments, the R^{A} is selected from hydrogen, deuterium, tritium, hydroxy, fluoro, chloro, bromo, cyano, C₁-C₅ alkyl (e.g., C₁-C₄ alkyl, C₁-C₃ alkyl), C₁-C₅ alkoxy (e.g., C₁-C₄ alkoxy, C₁-C₃ alkoxy), C₁-C₅ haloalkyl (e.g., C₁-C₄ haloalkyl, C₁-C₃ haloalkyl; e.g., C₁-C₅ fluoroalkyl, C₁-C₅ chloroalkyl, C₁-C₅ bromoalkyl), and 3-6 membered cycloalkyl (e.g., 3-5 membered cycloalkyl, 3-4 membered cycloalkyl).

In some embodiments, the R^{A} is selected from hydrogen, deuterium, tritium, hydroxy, fluoro, chloro, bromo, cyano, cyclopropyl, cyclobutyl, methyl, ethyl, propyl, butyl, pentyl, methoxy, ethoxy, propoxy, C₁-C₃ fluoroalkyl (e.g., trifluoromethyl, trifluoroethyl), C₁-C₃ chloroalkyl (e.g., trichloromethyl, trichloroethyl), and C₁-C₃ bromoalkyl (e.g., tribromomethyl, tribromoethyl); preferably hydrogen, deuterium, tritium, fluoro, chloro, bromo, cyano, cyclopropyl, cyclobutyl, methyl, ethyl, propyl, butyl, methoxy, ethoxy, and C₁-C₃ fluoroalkyl (e.g., trifluoromethyl, trifluoroethyl). In certain specific examples, the R^{A} is selected from cyclopropyl. In certain specific examples, the R^{A} is selected from bromo. In certain specific examples, the R^{A} is selected from trifluoromethyl. In certain specific examples, the R^{A} is selected from methoxy. In certain specific examples, the R^{A} is selected from hydrogen, deuterium, tritium, cyano, methyl, ethyl, and propyl.

Further, the R^{A} is selected from hydrogen, deuterium, tritium, hydroxy, halogen, cyano, cyclopropyl, cyclobutyl, methyl, ethyl, methoxy, ethoxy, and trifluoromethyl.

Further, the ring A is selected from a 6-10 membered aromatic ring, a 5-10 membered aromatic heterocycle, a 3-8 membered unsaturated aliphatic heterocycle, a 7-12 membered (e.g., 8-10 membered) fused ring consisted of an aromatic ring and an unsaturated aliphatic heterocycle, and a 7-12 membered fused ring consisted of an aromatic heterocycle and an unsaturated aliphatic heterocycle; and preferably, the aromatic ring and the aromatic heterocycle are a monocyclic ring or a bicyclic ring, the unsaturated aliphatic heterocycle is a monocyclic ring, the fused ring is a bicyclic ring, and the aromatic heterocycle, the unsaturated aliphatic heterocycle, and the fused ring each independently comprise 1-3 heteroatoms which are independently selected from N, O, and S.

In some embodiments, the ring A is selected from a 6-10 membered aromatic ring (e.g., a 6 membered aromatic ring, a 10 membered aromatic ring), a 5-10 membered aromatic heterocycle (e.g., a 5 membered aromatic heterocycle, a 6 membered aromatic heterocycle, a 9 membered aromatic heterocycle, a 10 membered aromatic heterocycle; comprising 1-2 heteroatoms selected from N, O, or S), a 4-7 membered unsaturated aliphatic heterocycle (e.g., comprising 1-2 heteroatoms selected from N, O, or S), a 8-12 membered fused ring (e.g., comprising 1-2 heteroatoms selected from N, O, or S) consisted of an aromatic ring and an unsaturated aliphatic heterocycle, and a 8-12 membered fused ring (e.g., comprising 1-2 heteroatoms selected from N, O, or S) consisted of an aromatic heterocycle and an unsaturated aliphatic heterocycle.

In some embodiments, the ring Ais selected from a 6-10 membered aromatic ring, a 5-10 membered aromatic heterocycle, a 5-6 membered unsaturated aliphatic heterocycle, and a 9-10 membered fused ring consisted of an aromatic ring and an unsaturated aliphatic heterocycle, and the aromatic heterocycle, the unsaturated aliphatic heterocycle, and the fused ring each independently comprise 1-2 heteroatoms selected from N, O, and S.

In some embodiments, the ring A is selected from

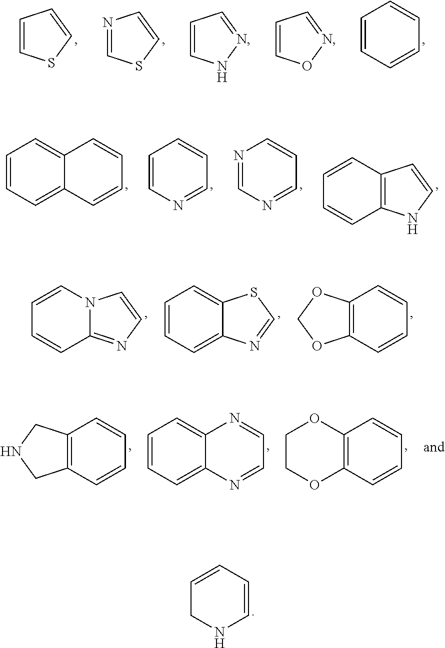

In some embodiments, o is selected from 0, 1, and 2, and R¹ is each independently selected from deuterium, tritium, hydroxy, halogen, cyano, =O, imino, an amine group, C₁-C₆ alkyl, C₁-C₆ halogenated alkyl, C₁-C₆ alkoxy, 4-8 membered cycloalkyl, and 4-8 membered saturated aliphatic heterocyclyl, and the R¹ is optionally substituted by C₁-C₆ alkyl.

In some embodiments, o is selected from 0, 1, and 2, and R¹ is each independently selected from deuterium, tritium, fluoro, chloro, bromo, cyano, =O, imino, an amine group, C₁-C₅ alkyl (e.g., C₁-C₄ alkyl, C₁-C₃ alkyl), C₁-C₅ halogenated alkyl (e.g., C₁-C₄ halogenated alkyl, C₁-C₃ halogenated alkyl), C₁-C₅ alkoxy (e.g., C₁-C₄ alkoxy, C₁-C₃ alkoxy), and 5-7 membered (e.g., 5-6 membered) saturated aliphatic heterocyclyl (e.g., comprising 1-2 heteroatoms selected from N, O, or S), and the imino, the amine group, and the saturated aliphatic heterocyclyl are optionally substituted by C₁-C₅ alkyl (e.g., C₁-C₄ alkyl, C₁-C₃ alkyl).

In some embodiments, o is selected from 0, 1, and 2, and R¹ is each independently selected from fluoro, chloro, bromo, cyano, =O, an amine group, methyl, ethyl, propyl, methoxy, ethoxy, propoxy, and 5-7 membered (e.g., 5-6 membered) saturated aliphatic heterocyclyl comprising 1-2 heteroatoms selected from N or 0, and the amine group is optionally substituted by methyl, ethyl, or propyl (e.g., methyl).

In some embodiments, o is selected from 0, 1, and 2, and R¹ is each independently selected from fluoro, chloro, bromo, cyano, =O, an amine group, methyl, ethyl, propyl, methoxy, ethoxy, propoxy, morpholinyl, thiomorpholinyl, piperidinyl, and piperazinyl (e.g., R¹ is each independently selected from fluoro, chloro, bromo, cyano, =O, an amine group, methyl, ethyl, propyl, methoxy, ethoxy, propoxy, and morpholinyl), and the amine group is optionally substituted by methyl, ethyl, or propyl (e.g., methyl).

In some embodiments, o is selected from 0, 1, and 2, and R¹ is each independently selected from fluoro, chloro, bromo, cyano, ═O, —NH₂, —NHCH₃, —N(CH₃)₂, —NHCH₂CH₃, —N(CH₂CH₃)₂, methyl, ethyl, propyl, methoxy, ethoxy, propoxy, and morpholinyl.

In an embodiment of the present application, the present application further provides a compound represented by formula (II), a stereoisomer, tautomer or mixture form thereof, or a pharmaceutical acceptable salt thereof, or a solvate (e.g., a hydrate) thereof, or a prodrug thereof,

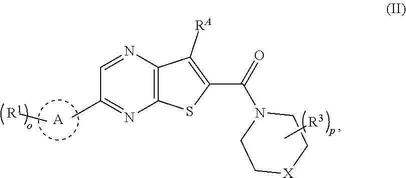

wherein ring A is selected from an aromatic ring, an aromatic heterocycle, an unsaturated aliphatic heterocycle, a fused ring consisted of an aromatic ring and an unsaturated aliphatic heterocycle, and a fused ring consisted of an aromatic heterocycle and an unsaturated aliphatic heterocycle;
R¹ is each independently selected from deuterium, tritium, nitro, hydroxy, mercapto, halogen, cyano, =O, imino, an amine group, an ester group, an aldehyde group, carboxyl, amido, C₁-C₆ alkyl, C₁-C₆ alkoxy, 3-8 membered cycloalkyl, and 3-8 membered saturated aliphatic heterocyclyl;
R^{A} is selected from hydrogen, deuterium, tritium, hydroxy, halogen, cyano, C₁-C₆ alkyl, C₁-C₆ alkoxy, C₁-C₆ haloalkyl, and 3-8 membered cycloalkyl;
o is selected from 0, 1, 2, and 3;
X is selected from a covalent bond, S, NH, CH₂, (CH₂)₂, or (CH₂)₃; R³ is each independently selected from deuterium, tritium, nitro, hydroxy, mercapto, cyano, halogen, an amine group, an ester group, an aldehyde group, carboxyl, amido, C₁-C₆ alkyl, C₁-C₆ haloalkyl, C₁-C₆ alkoxy, C₃-C₈ cycloalkyl, 6-10 membered aryl, and 5-10 membered heteroaryl; p is selected from 0 and 1; and
preferably, the R³ is each independently selected from deuterium, tritium, nitro, hydroxy, mercapto, cyano, fluoro, chloro, bromo, an amine group, an ester group, an aldehyde group, carboxyl, amido, methyl, ethyl, n-propyl, isopropyl, methoxy, ethoxy, n-propoxy, isopropoxy, trifluoromethyl, trifluoroethyl, trichloromethyl, trichloroethyl, cyclobutyl, cyclopropyl, phenyl, and pyridyl.

Further, in embodiments of the present application, the p is 0.

Further, in embodiments of the present application, the p is 0, and the X is CH₂.

In embodiments of the present application, the R^{A} is selected from hydrogen, deuterium, tritium, hydroxy, halogen, cyano, cyclopropyl, cyclobutyl, methyl, ethyl, methoxy, ethoxy, and trifluoromethyl. In certain specific embodiments, the R^{A} is selected from hydrogen, deuterium, tritium, cyano, cyclopropyl, trifluoromethyl, halogen, and methyl.

In some embodiments, the R^{A} is selected from hydrogen, deuterium, tritium, hydroxy, fluoro, chloro, bromo, cyano, cyclopropyl, cyclobutyl, methyl, ethyl, propyl, butyl, pentyl, methoxy, ethoxy, propoxy, C₁-C₃ fluoroalkyl (e.g., trifluoromethyl, trifluoroethyl), C₁-C₃ chloroalkyl (e.g., trichloromethyl, trichloroethyl), and C₁-C₃ bromoalkyl (e.g., tribromomethyl, tribromoethyl); preferably hydrogen, deuterium, tritium, fluoro, chloro, bromo, cyano, cyclopropyl, cyclobutyl, methyl, ethyl, propyl, butyl, methoxy, ethoxy, and C₁-C₃ fluoroalkyl (e.g., trifluoromethyl, trifluoroethyl). In certain specific embodiments, the R^{A} is selected from cyclopropyl. In certain specific embodiments, the R^{A} is selected from bromo. In certain specific embodiments, the R^{A} is selected from trifluoromethyl. In certain specific embodiments, the R^{A} is selected from methoxy. In certain specific embodiments, the R^{A} is selected from hydrogen, deuterium, tritium, cyano, methyl, ethyl, and propyl.

In embodiments of the present application, the ring A is selected from a 6-10 membered aromatic ring, a 5-10 membered aromatic heterocycle, a 3-8 membered unsaturated aliphatic heterocycle, a 7-12 membered fused ring consisted of an aromatic ring and an unsaturated aliphatic heterocycle, and a 7-12 membered fused ring consisted of an aromatic heterocycle and an unsaturated aliphatic heterocycle.

Further, the aromatic ring and the aromatic heterocycle are preferably a monocyclic ring or a bicyclic ring, the unsaturated aliphatic heterocycle is preferably a monocyclic ring, the fused ring is preferably a bicyclic ring, the aromatic heterocycle, the unsaturated aliphatic heterocycle, and the fused ring each independently comprise 1-3 heteroatoms, and the heteroatom is independently selected from N, O, and S.

In some embodiments, the ring A is selected from a 6-10 membered aromatic ring (e.g., a 6 membered aromatic ring, a 10 membered aromatic ring), a 5-10 membered aromatic heterocycle (e.g., a 5 membered aromatic heterocycle, a 6 membered aromatic heterocycle, a 9 membered aromatic heterocycle, a 10 membered aromatic heterocycle; comprising 1-2 heteroatoms selected from N, O, or S), a 4-7 membered unsaturated aliphatic heterocycle (e.g., comprising 1-2 heteroatoms selected from N, O, or S), a 8-12 membered fused ring (e.g., comprising 1-2 heteroatoms selected from N, O, or S) consisted of an aromatic ring and an unsaturated aliphatic heterocycle, and a 8-12 membered fused ring (e.g., comprising 1-2 heteroatoms selected from N, O, or S) consisted of an aromatic heterocycle and an unsaturated aliphatic heterocycle.

In some embodiments, the ring Ais selected from a 6-10 membered aromatic ring, a 5-10 membered aromatic heterocycle, a 5-6 membered unsaturated aliphatic heterocycle, and a 9-10 membered fused ring consisted of an aromatic ring and an unsaturated aliphatic heterocycle, and the aromatic heterocycle, the unsaturated aliphatic heterocycle, and the fused ring each independently comprise 1-2 heteroatoms selected from N, O, and S.

In certain embodiments of the present application, the ring A is selected from

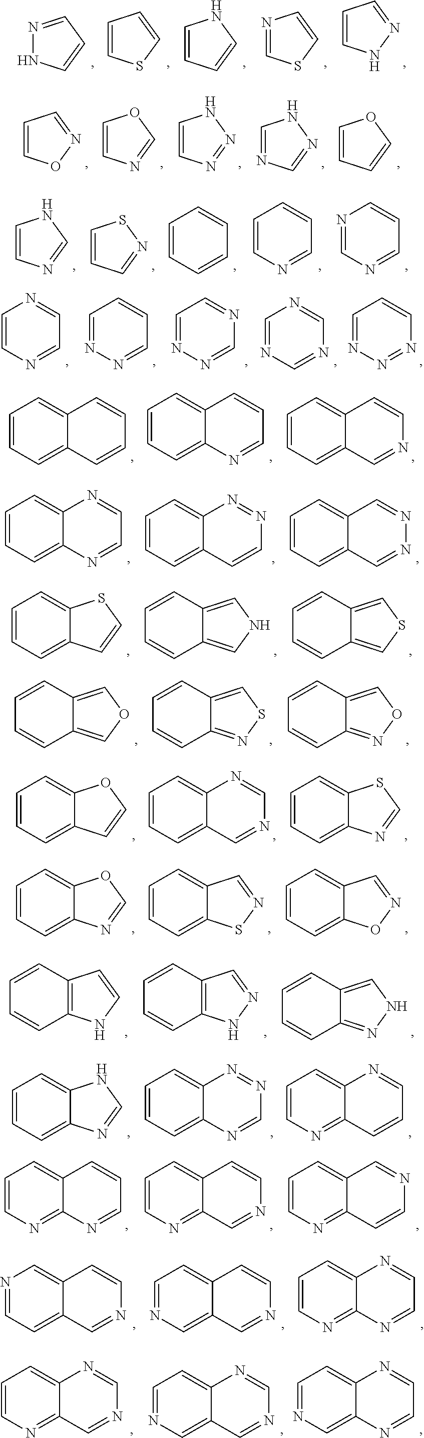

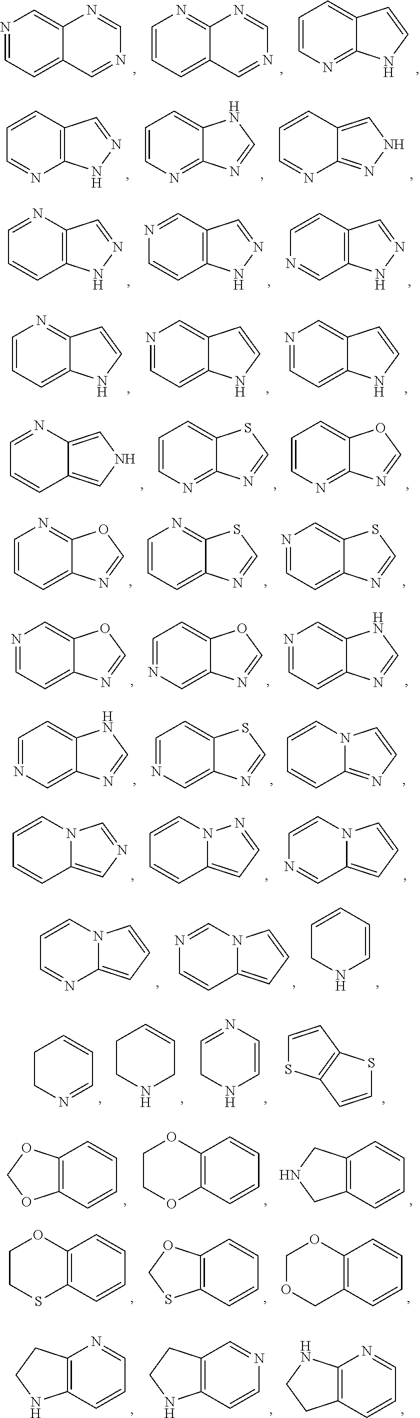

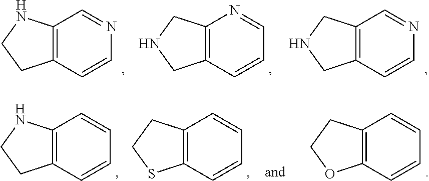

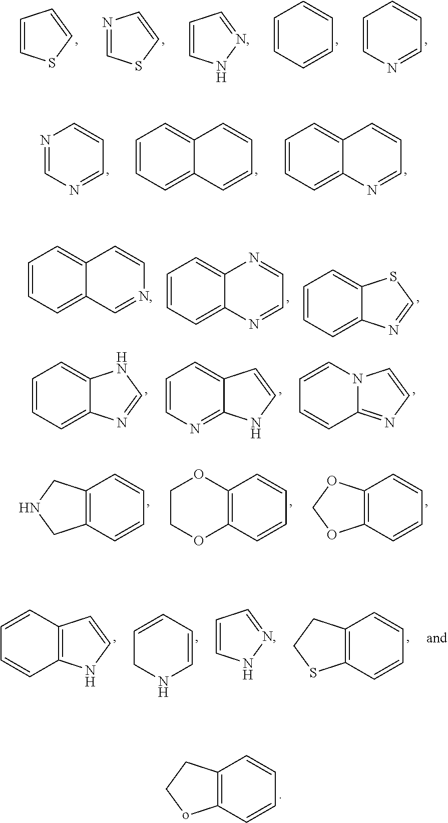

In certain specific embodiments of the present application, the ring A is preferably selected from

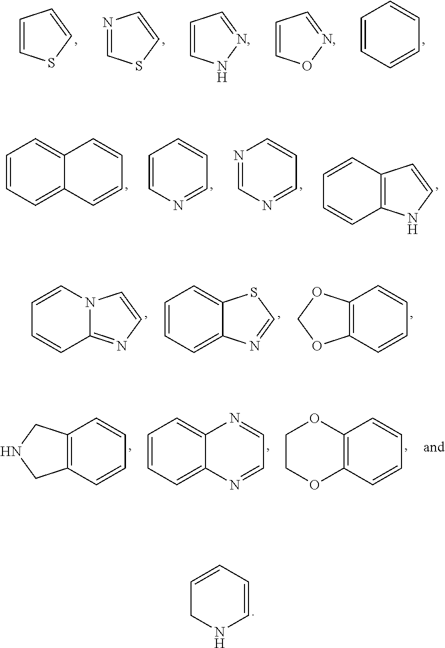

In certain embodiments of the present application, the R¹ according to the present application is each independently selected from deuterium, tritium, nitro, hydroxy, mercapto, halogen, cyano, ═O, imino, an amine group, an ester group, an aldehyde group, carboxyl, amido, cyclopropyl, cyclopropylmethyl, cyclobutyl, trifluoromethyl, cyclohexyl, cyclopentyl, methyl, ethyl, isopropyl, n-butyl, isobutyl, tert-butyl, n-pentyl, isopentyl, tert-pentyl, n-hexyl, morpholinyl, thiomorpholinyl, piperidinyl, piperazinyl, oxazolidinyl, isoxazolidinyl, thiazolidinyl, isothiazolidinyl, dioxolanyl, dioxanyl, methoxy, ethoxy, n-propoxy, isopropoxy, cyclopropoxy, cyclopropylmethoxy, n-butoxy, isobutoxy, tert-butoxy, n-pentyloxy, isopentyloxy, tert-pentyloxy, and n-hexyloxy, wherein the R¹ is optionally substituted by one or more independently selected from deuterium, tritium, nitro, hydroxy, —NH₂, mercapto, halogen, cyano, an ester group, carboxyl, amido, =O, =NH, C₁-C₆ alkyl, C₁-C₆ halogenated alkyl, C₁-C₆ alkoxy, C₁-C₆ halogenated alkyl, 3-8 membered cycloalkyl, 6-10 membered aryl, 5-10 membered aliphatic heterocyclyl, and 5-10 membered heteroaryl.

In certain specific embodiments of the present application, the R¹ according to the present application is each independently and preferably selected from deuterium, tritium, nitro, hydroxy, mercapto, cyano, =O, =NH, —NH₂, —N(CH₃)₂, —NHCH₃, ═NCH₃, an ester group, an aldehyde group, carboxyl, amido, cyclopropyl, cyclobutyl, cyclohexyl, cyclopentyl, methyl, ethyl, isopropyl, cyclopropylmethyl, trifluoromethyl, trifluoroethyl, trichloromethyl, trichloroethyl, morpholinyl, piperidinyl, N-methylpiperazinyl, p-methylpiperidinyl, piperazinyl, methoxy, ethoxy, isopropoxy, cyclopropoxy, cyclopropylmethoxy, halogen, and cyclopropoxy.

In some embodiments, the present application provides a compound represented by formula (I), a stereoisomer or mixture form thereof, or a pharmaceutically acceptable salt thereof, or a solvate (e.g., a hydrate) thereof, or a prodrug thereof,
wherein the ring A is selected from a 6-10 membered aromatic ring, a 5-10 membered aromatic heterocycle comprising 1-2 heteroatoms selected from N, O, or S, a 4-7 membered unsaturated aliphatic heterocycle comprising 1-2 heteroatoms selected from N, O, or S, a 8-12 membered fused ring (comprising 1-2 heteroatoms selected from N, O, or S) consisted of an aromatic ring and an unsaturated aliphatic heterocycle, and a 8-12 membered fused ring (comprising 1-2 heteroatoms selected from N, O, or S) consisted of an aromatic heterocycle and an unsaturated aliphatic heterocycle; preferably, the ring A is selected from a 6-10 membered aromatic ring, a 5-10 membered aromatic heterocycle, a 5-6 membered unsaturated aliphatic heterocycle, and a 9-10 membered fused ring consisted of an aromatic ring and an unsaturated aliphatic heterocycle, and the aromatic heterocycle, the unsaturated aliphatic heterocycle, and the fused ring each independently comprise 1-2 heteroatoms selected from N, O, and S;
the ring B is a 5-10 membered, e.g., 5-8 membered or 5-7 membered, saturated aliphatic heterocycle comprising at least 1 nitrogen atom; preferably, the ring B is selected from

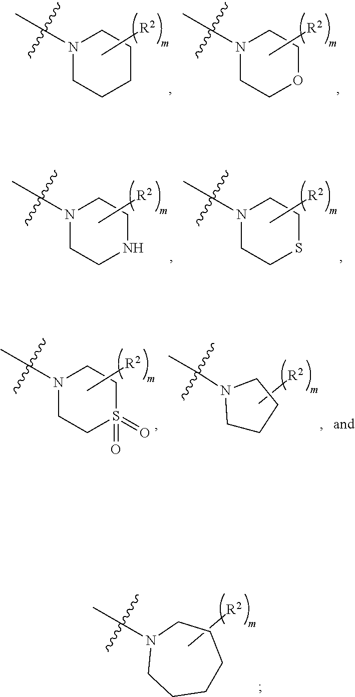

m is selected from 0, 1, 2, and 3; R² is each independently selected from deuterium, tritium, nitro, hydroxy, mercapto, cyano, halogen, an amine group, an ester group, an aldehyde group, carboxyl, amido, C₁-C₆ alkyl, C₁-C₆ haloalkyl, C₁-C₆ alkoxy, 3-8 membered cycloalkyl, 6-10 membered aryl, and 5-10 membered heteroaryl; the R^{A} is selected from hydrogen, deuterium, tritium, hydroxy, fluoro, chloro, bromo, cyano, C₁-C₅ alkyl, C₁-C₅ alkoxy, C₁-C₅ haloalkyl, and 3-6 membered cycloalkyl; preferably, the R^{A} is selected from hydrogen, deuterium, tritium, hydroxy, fluoro, chloro, bromo, cyano, cyclopropyl, cyclobutyl, methyl, ethyl, propyl, butyl, pentyl, methoxy, ethoxy, propoxy, C₁-C₃ fluoroalkyl, C₁-C₃ chloroalkyl, and C₁-C₃ bromoalkyl;
is selected from 0, 1, and 2, and R¹ is each independently selected from deuterium, tritium, hydroxy, halogen, cyano, =O, imino, an amine group, C₁-C₆ alkyl, C₁-C₆ halogenated alkyl, C₁-C₆ alkoxy, 4-8 membered cycloalkyl, and 4-8 membered saturated aliphatic heterocyclyl, wherein the R¹ is optionally substituted by C₁-C₆ alkyl; and preferably, o is selected from 0, 1, and 2, and R¹ is each independently selected from deuterium, tritium, fluoro, chloro, bromo, cyano, =O, imino, an amine group, C₁-C₅ alkyl, C₁-C₅ halogenated alkyl, C₁-C₅ alkoxy, and 5-7 membered saturated aliphatic heterocyclyl, wherein the imino and the amine group are optionally substituted by C₁-C₅ alkyl.

In some embodiments, the present application provides a compound represented by formula (I), a stereoisomer or mixture form thereof, or a pharmaceutically acceptable salt thereof, or a solvate (e.g., a hydrate) thereof, or a prodrug thereof, wherein the ring A is selected from

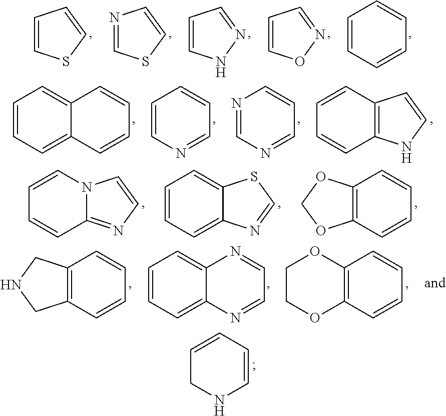

the ring B is selected from

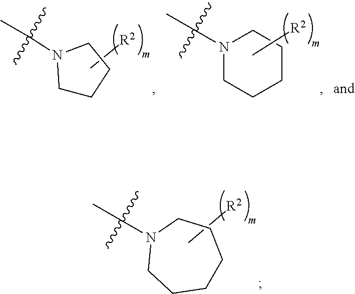

m is selected from 0 and 1;
R² is each independently selected from deuterium, tritium, hydroxy, fluoro, chloro, bromo, an amine group, methyl, and ethyl; preferably deuterium and tritium;
the R^{A} is selected from hydrogen, deuterium, tritium, fluoro, chloro, bromo, cyano, cyclopropyl, cyclobutyl, methyl, ethyl, propyl, butyl, methoxy, ethoxy, and C₁-C₃ fluoroalkyl (e.g., trifluoromethyl, trifluoroethyl); preferably hydrogen, bromo, cyano, cyclopropyl, methyl, ethyl, propyl, methoxy, and trifluoromethyl; o is selected from 0, 1, and 2, and R¹ is each independently selected from fluoro, chloro, bromo, cyano, =O, an amine group, methyl, ethyl, propyl, methoxy, ethoxy, propoxy, morpholinyl, thiomorpholinyl, piperidinyl, and piperazinyl, wherein the amine group is optionally substituted by methyl, ethyl, or propyl (e.g., methyl).

In some specific embodiments of the present application, the present application provides compounds shown below, stereoisomers, tautomers or mixture forms thereof, or pharmaceutically acceptable salts thereof, or solvates (e.g., hydrates) thereof, or prodrugs thereof:

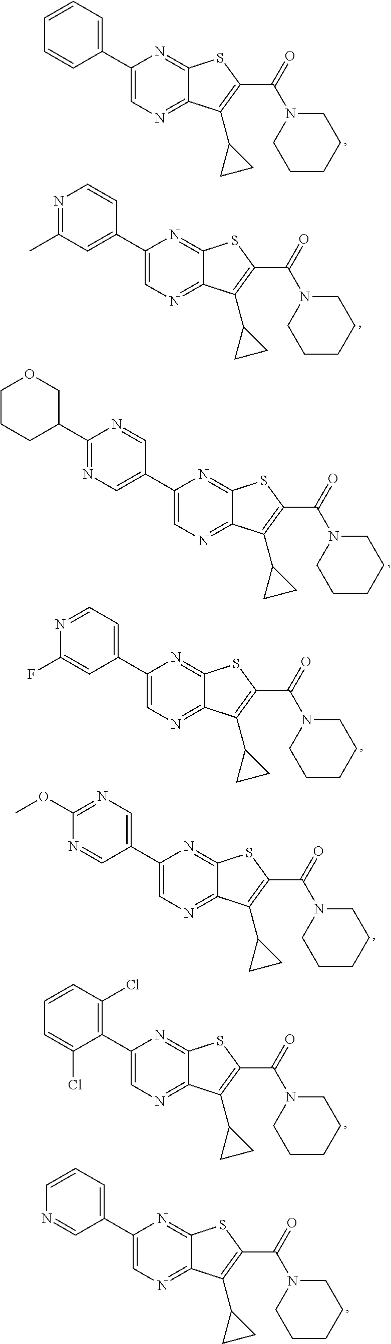

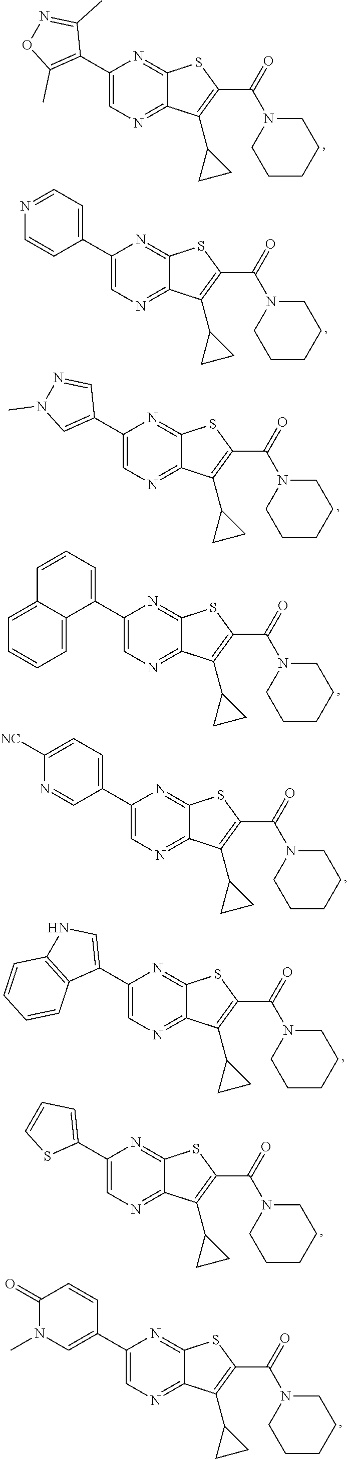

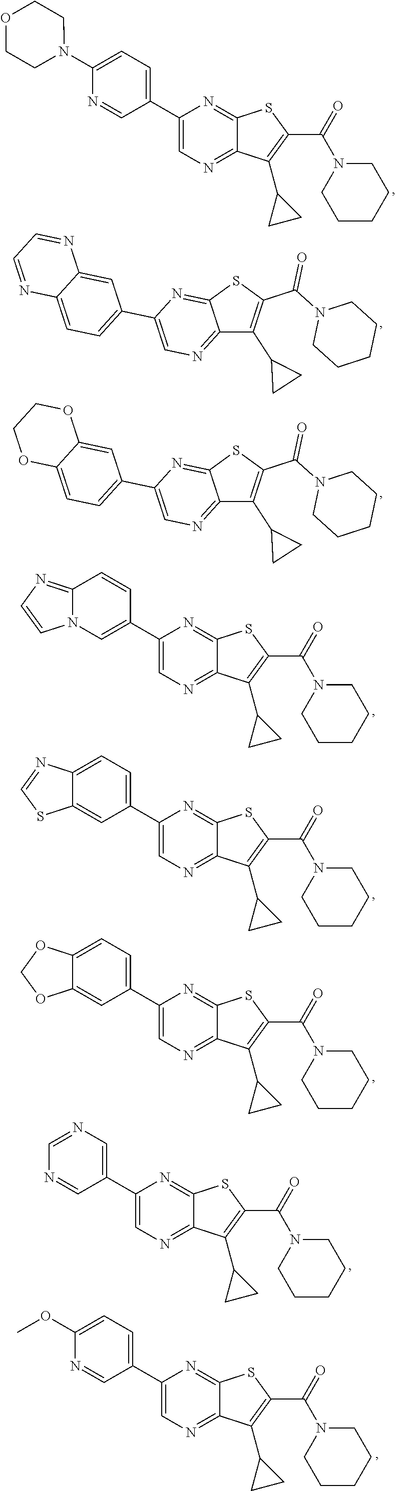

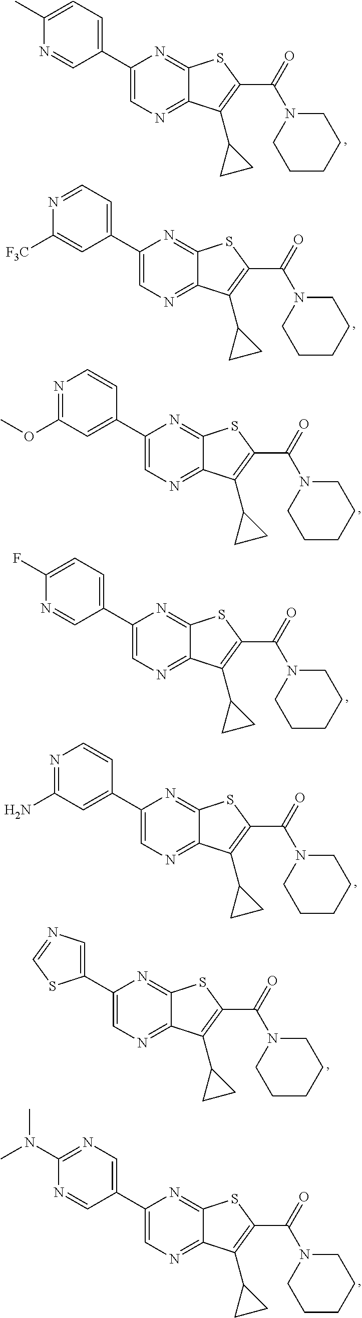

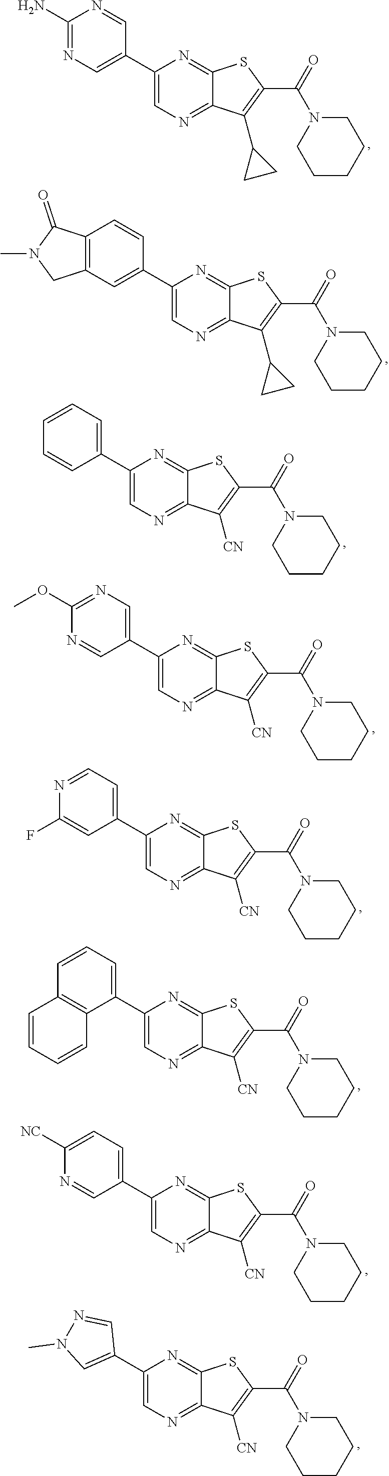

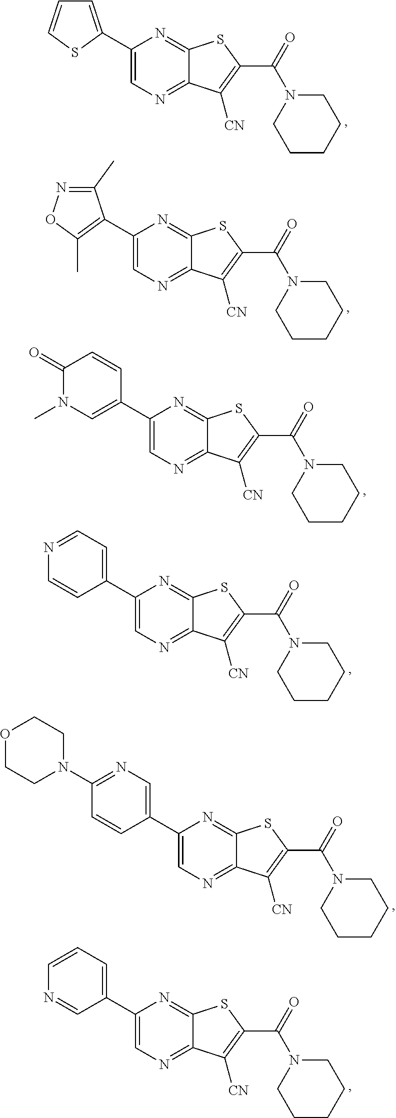 [SMILES: N#Cc1c(C(=O)N2CCCCC2)sc2nc(-c3cccs3)cnc12 ; Cc1noc(C)c1-c1cnc2c(C#N)c(C(=O)N3CCCCC3)sc2n1 ; Cn1cc(-c2cnc3c(C#N)c(C(=O)N4CCCCC4)sc3n2)ccc1=O ; N#Cc1c(C(=O)N2CCCCC2)sc2nc(-c3ccncc3)cnc12 ; N#Cc1c(C(=O)N2CCCCC2)sc2nc(-c3ccc(N4CCOCC4)nc3)cnc12 ; N#Cc1c(C(=O)N2CCCCC2)sc2nc(-c3cccnc3)cnc12]

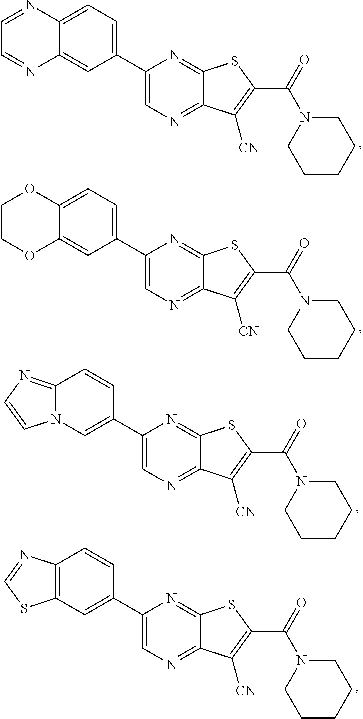 [SMILES: N#Cc1c(C(=O)N2CCCCC2)sc2nc(-c3ccc4nccnc4c3)cnc12 ; N#Cc1c(C(=O)N2CCCCC2)sc2nc(-c3ccc4c(c3)OCCO4)cnc12 ; N#Cc1c(C(=O)N2CCCCC2)sc2nc(-c3ccc4nccn4c3)cnc12 ; N#Cc1c(C(=O)N2CCCCC2)sc2nc(-c3ccc4ncsc4c3)cnc12]

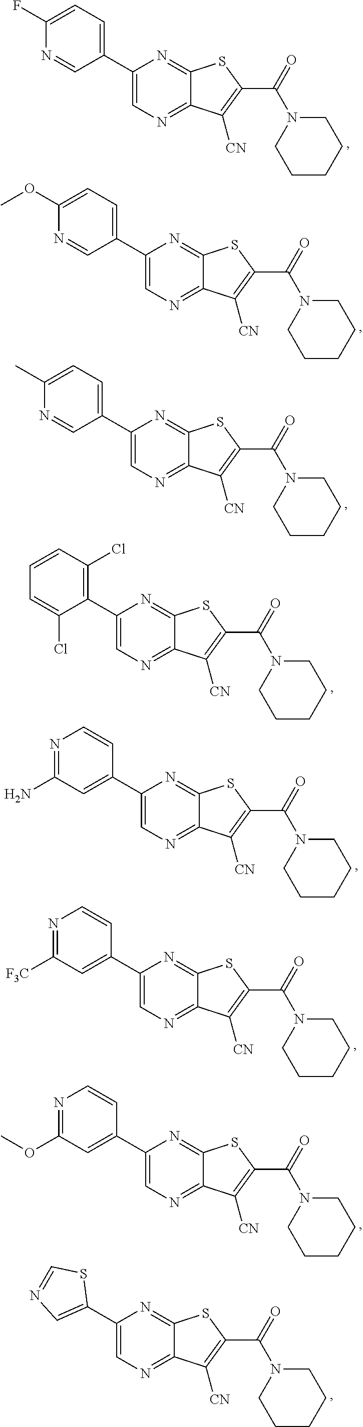

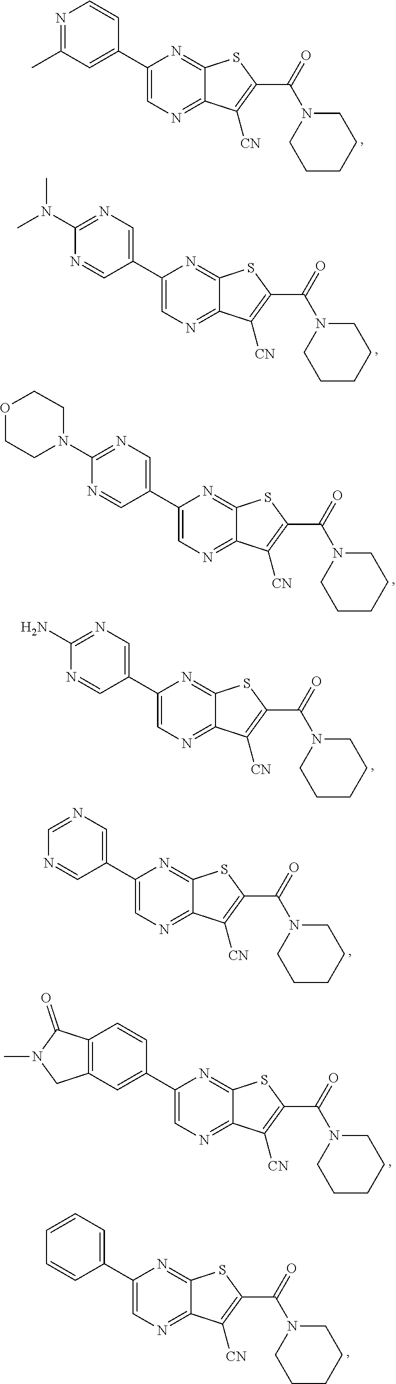

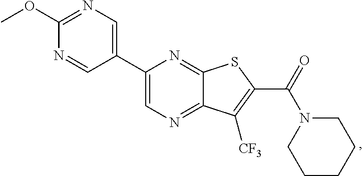 [SMILES: COc1ncc(-c2cnc3c(C(F)(F)F)c(C(=O)N4CCCCC4)sc3n2)cn1]

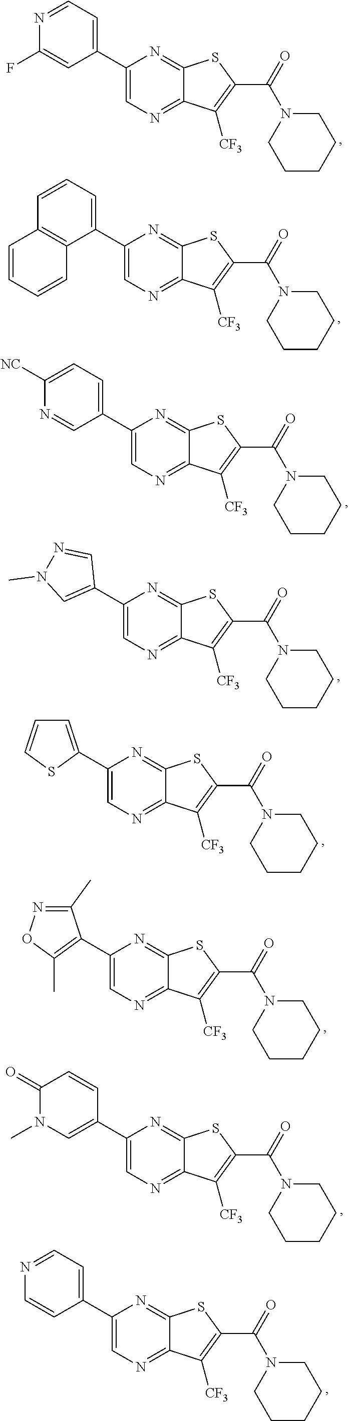

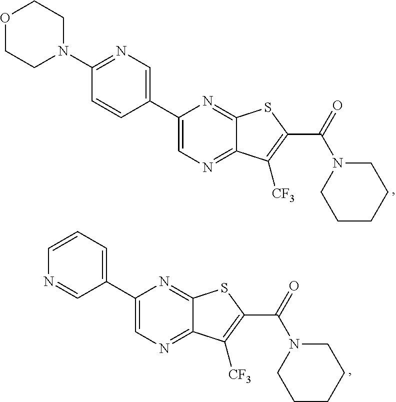 [SMILES: O=C(c1sc2nc(-c3ccc(N4CCOCC4)nc3)cnc2c1C(F)(F)F)N1CCCCC1 ; O=C(c1sc2nc(-c3cccnc3)cnc2c1C(F)(F)F)N1CCCCC1]

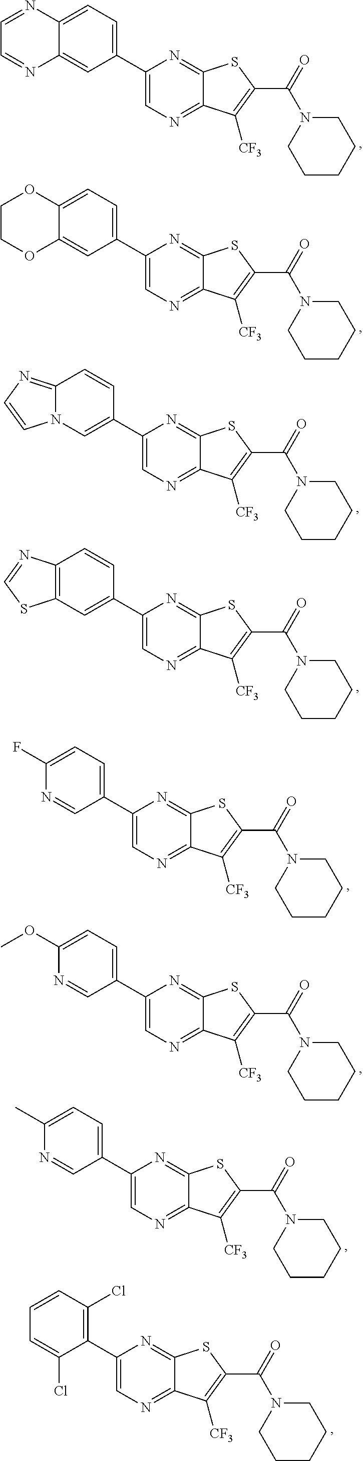

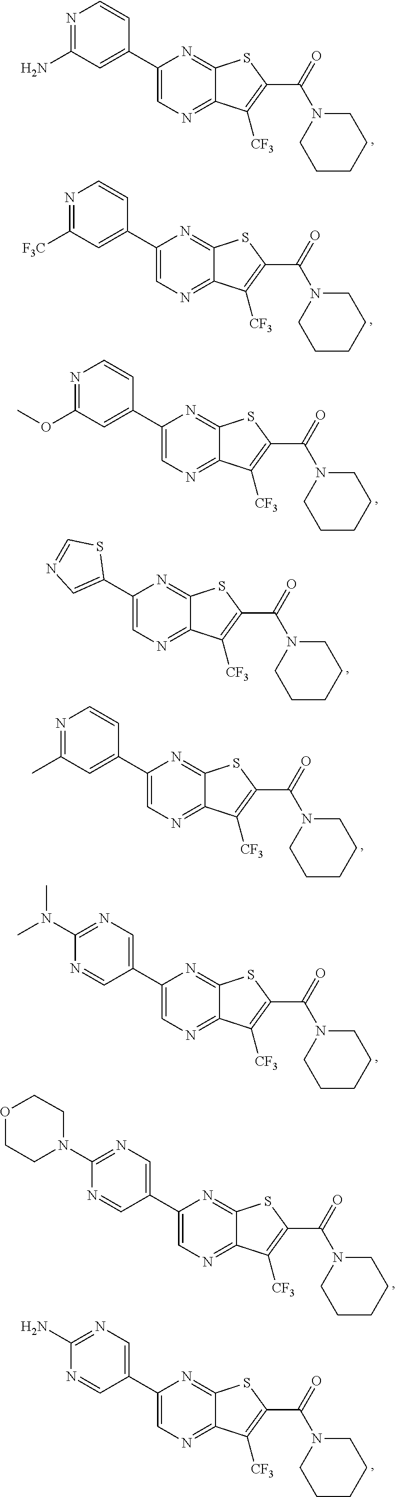

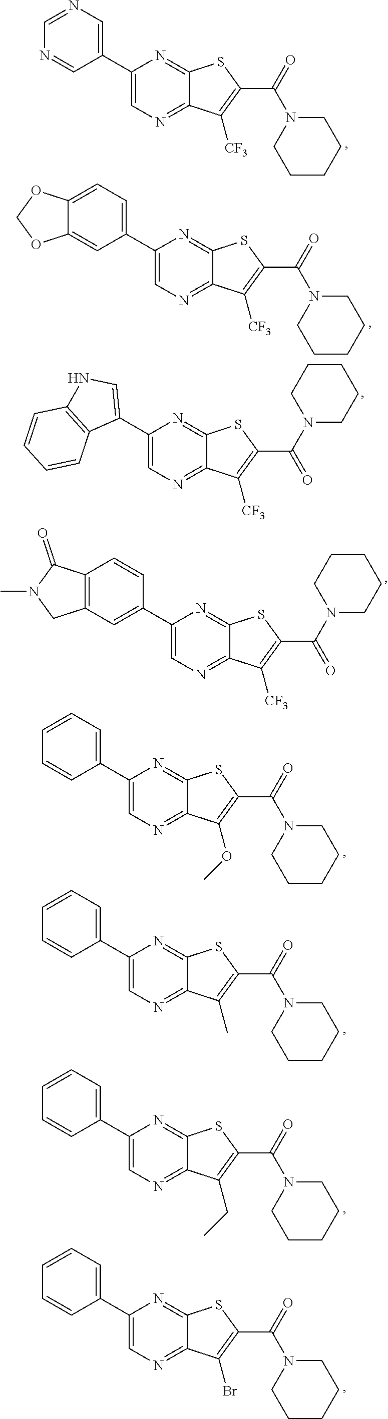

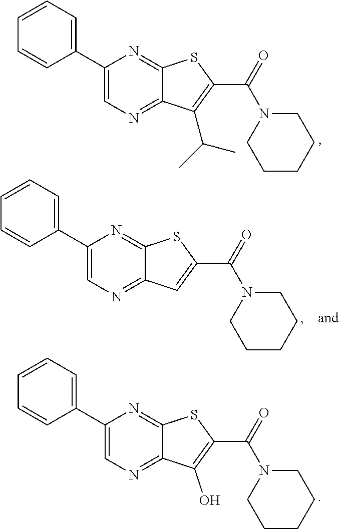 [SMILES: CC(C)c1c(C(=O)N2CCCCC2)sc2nc(-c3ccccc3)cnc12 ; O=C(c1cc2ncc(-c3ccccc3)nc2s1)N1CCCCC1 ; O=C(c1sc2nc(-c3ccccc3)cnc2c1O)N1CCCCC1]

The present application further covers embodiments that are obtained by any combination, deletion or conversion of the above embodiments.

Another aspect of the present application provides a pharmaceutical composition, comprising at least one of the above-mentioned compounds, the stereoisomers, tautomers or mixture forms thereof, or the pharmaceutically acceptable salts thereof, or the solvates thereof, or the prodrugs thereof, and at least one pharmaceutically acceptable excipient.

Another aspect of the present application provides use of the above-mentioned compounds, or the stereoisomers, tautomers or mixture forms thereof, or the pharmaceutically acceptable salts thereof, or the solvates thereof, or the prodrugs thereof, or the pharmaceutical composition in the preparation of a medicament. Wherein the medicament is a 15-PGDH inhibitor which can be used for treating a disease associated with an undesirably increased activity level of 15-PGDH. Alternatively, the present application provides the above-mentioned compounds, or the stereoisomers, tautomers or mixture forms thereof, or the pharmaceutically acceptable salts thereof, or the solvates thereof, or the prodrugs thereof, or the pharmaceutical composition for use as a medicament. Alternatively, the present application provides a method of treating or preventing a disease associated with 15-PGDH, comprising administering to a subject in need thereof the above-mentioned compounds, or the stereoisomers, tautomers or mixture forms thereof, or the pharmaceutically acceptable salts thereof, or the solvates thereof, or the prodrugs thereof, or the pharmaceutical composition. The disease associated with 15-PGDH herein refers to a disease or complication thereof for which a clinically beneficial effect, such as remission, amelioration, cessation of progression, alleviation, or no further deterioration, is achieved by inhibiting the activity of 15-PGDH.

In certain specific embodiments, the medicament, the inhibitor or the method is used for treating or preventing fibrosis, oral ulcer, gum disease, colitis, ulcerative colitis, gastroduodenal ulcer, inflammatory disease, vascular insufficiency, Raynaud's disease, Buerger's disease, neuropathy, pulmonary arterial hypertension, cardiovascular and renal disease, cardiovascular disease, trauma, skin damage, autoimmune disease, graft-versus-host disease, osteoporosis, ear disease, eye disease, neutropenia, diabetes mellitus, underactive bladder, or for promoting hair growth, pigmentation, tissue repair, tissue regeneration, implant in stem cell transplantation or bone marrow transplantation or organ transplantation, neurogenesis and neuronal cell death, muscle regeneration, and cervical ripening, or for enhancing resistance to the toxicity of radiation exposure, the toxicity of chemotherapy and the toxicity of immunosuppressant.

### Definition

Unless otherwise stated, the following terms used in the specification and claims have the following meanings. A particular term or phrase shall not be considered uncertain or unclear in the absence of a specific definition, but should be understood according to its ordinary meaning.

“Alkyl” refers to a saturated aliphatic hydrocarbon group. The alkyl moiety may be a linear or branched alkyl; C₁-C₆ alkyl used herein refers to a linear or branched alkyl comprising 1 to 6 (e.g., 1, 2, 3, 4, 5, 6, or a range value composed of any two of the preceding numerical values) carbon atoms. Typical alkyl includes, but is not limited to, methyl, ethyl, n-propyl, isopropyl, n-butyl, isobutyl, t-butyl, n-pentyl, isopentyl, tert-amyl, n-hexyl, etc.

“Alkoxy” refers to —O-alkyl; C₁-C₆ alkoxy used herein refers to a linear or branched alkoxy group comprising 1 to 6 (e.g., 1, 2, 3, 4, 5, 6, or a range value composed of any two of the preceding numerical values) carbon atoms. Typical alkoxy includes, but is not limited to, methoxy, ethoxy, n-propoxy, isopropoxy, n-butoxy, isobutoxy, t-butoxy, n-pentyloxy, isopentyloxy, tert-pentyloxy, n-hexyloxy, etc.

“Ring” refers to any cyclic covalently closed structure, including, for example, a carbocycle (e.g., an aromatic or alicyclic ring), or a heterocycle (e.g., an aromatic or aliphatic heterocycle). The carbocycle refers to a ring only consisted of carbon atoms, and the heterocycle refers to a closed structure formed by covalently bonding carbon atoms and heteroatoms. Depending on the number of the ring, the “ring” may be monocyclic, bicyclic, tricyclic or polycyclic. When the ring is a bicyclic, tricyclic or polycyclic ring, the relationship between individual rings may include a fused ring, a spirocycle, or a bridged cycle.

“Heteroatom” refers to any atom, other than a carbon atom, that can be covalently bonded to a carbon atom. Common heteroatoms include, but are not limited to, O, S, N, P, Si, etc.

The “membered” refers to the number of skeleton atoms constituting a ring. A typical 5 membered ring may include, but is not limited to, cyclopentane, pyrrole, imidazole, thiazole, furan, thiophene, and the like, and a typical 6 membered ring includes, but is not limited to, cyclohexane, pyridine, pyran, pyrazine, thiapyran, pyridazine, pyrimidines, benzene, etc.

“Alicyclic ring” or “alicyclic group” refers to a saturated or partially unsaturated non-aromatic cyclic group with a skeleton consisted of carbon atoms; the saturated carbocycle may be referred to as, e.g., a saturated alicyclic ring; the partially unsaturated carbocycle may be referred to as, e.g., an unsaturated alicyclic ring; an alicyclic ring may be consisted of 3-10 (e.g., 3, 4, 5, 6, 7, 8, 9 or 10) atoms, and may be a monocyclic ring or a polycyclic ring; for example, a 3-8 membered alicyclic group refers to an alicyclic group consisted of 3-8 skeleton atoms. A typical alicyclic structure includes, but is not limited to:

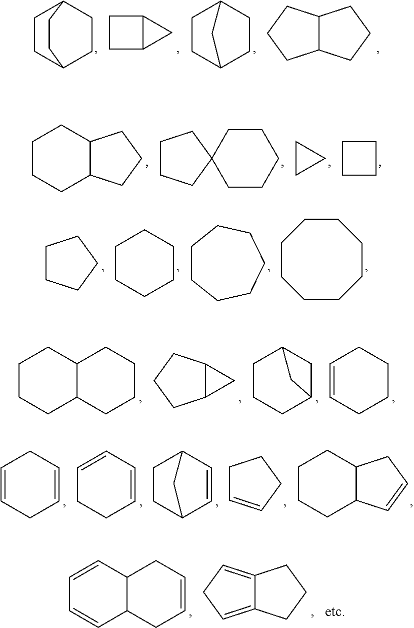

“Aliphatic heterocycle” or “aliphatic heterocyclic group” refers to a nonaromatic cyclic group formed by replacing carbon atom(s) in an alicyclic ring with one or more heteroatoms. The aliphatic heterocycle or aliphatic heterocyclic group may include a saturated aliphatic heterocycle and an unsaturated aliphatic heterocycle. For example, a 3-8 membered aliphatic heterocyclic group used in the present application refers to a nonaromatic cyclic group comprising one or more heteroatoms consisted of 3-8 skeleton atoms, and may be a saturated aliphatic heterocyclic group and an unsaturated aliphatic heterocyclic group.

“Saturated aliphatic heterocycle” or “saturated aliphatic heterocyclic group” means that the atoms in the aliphatic heterocycle that form the ring skeleton are all saturated. For example, a 3-12 membered saturated aliphatic heterocycle used in the present application refers to a nonaromatic cyclic group formed by 3-12 atoms constituting the ring skeleton, wherein the atoms constituting the ring skeleton comprise saturated carbon atoms and heteroatoms. A typical saturated aliphatic heterocycle includes, but is not limited to:

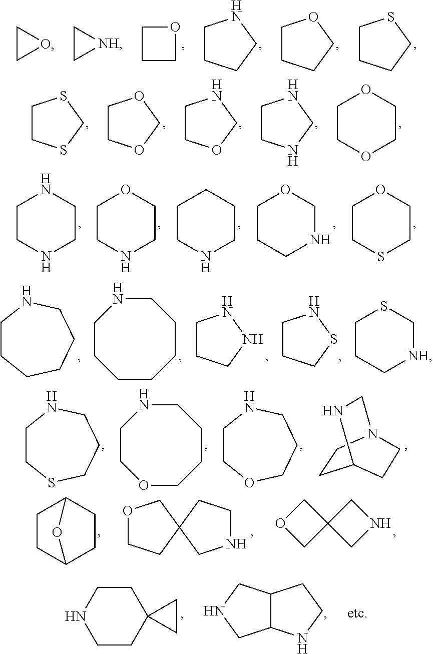

“Unsaturated aliphatic heterocycle” or “unsaturated aliphatic heterocyclyl” refers to a non-aromatic cyclic structure containing some unsaturated atoms as a ring skeleton in an aliphatic heterocycle. The “unsaturated aliphatic heterocycle” in the present application means that the skeleton of the aliphatic heterocycle contains unsaturated carbon atoms. A 3-8 membered unsaturated aliphatic heterocycle used in the present application refers to a nonaromatic cyclic group formed by 3-8 (e.g., 3, 4, 5, 6, 7 or 8) skeleton atoms, wherein the atoms constituting the ring skeleton include saturated carbon atoms, unsaturated carbon atoms, and heteroatoms, and a typical unsaturated aliphatic heterocycle includes, but is not limited to:

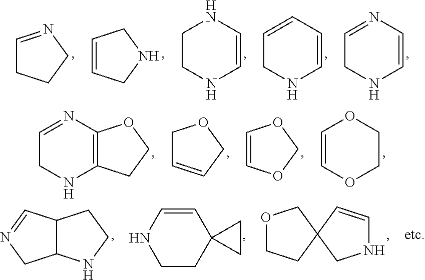

“Cycloalkyl” refers to a saturated aliphatic carbocyclic group, and may also be referred to as, for example, a saturated alicyclic ring. The cycloalkyl group may be a monocyclic ring, a spirocycle, a fused ring or a bridged cycle. A 3-8 membered cycloalkyl used in the application refers to a cyclic alkyl comprising 3-8 carbon atoms. A typical cycloalkyl includes, but is not limited to, cyclopropyl, cyclobutyl, cyclopentyl, cyclohexyl, bicyclo[2,1,1]hexyl, cycloheptyl, and the like.

“Aromatic ring” or “aryl” refers to a completely unsaturated carbocycle with a planar ring having a delocalized π-electron system and containing 4n+2π electrons, where n is an integer. The aromatic ring may consist of six, eight, ten, or more than ten carbon atoms, and may be a monocyclic ring, a bicyclic ring, a tricyclic ring, or a polycyclic ring. Common aromatic ring includes, but is not limited to, benzene ring, naphthalene ring, phenanthrene ring, anthracene ring, tetrabenzene, pyrene ring, pentabenzene, and the like. As used in the present application, a 6-10 membered aromatic ring or a 6-10 membered aryl group refers to an aromatic ring group consisting of 6-10 skeleton carbon atoms.

“Aromatic heterocycle” or “heteroaryl” refers to an aromatic cyclic structure formed by replacing carbon atoms in the aromatic ring with one or more heteroatoms, and a typical aromatic heterocycle or heteroaryl includes, but is not limited to:

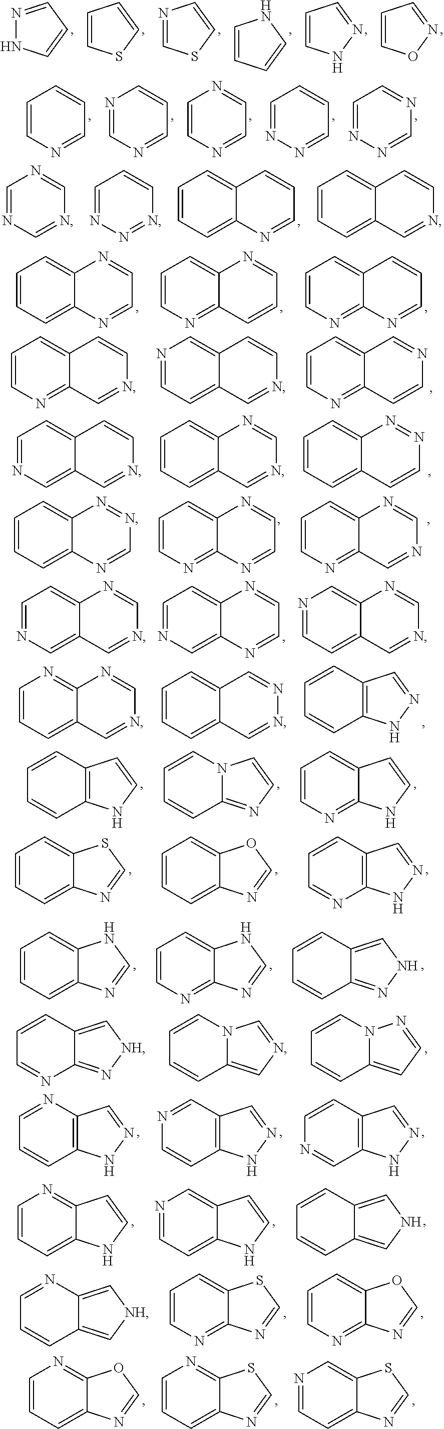

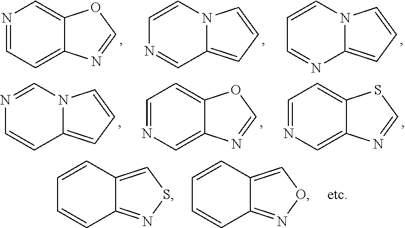

A 5-10 membered aromatic heterocycle or 5-10 membered heteroaryl used in the present application refers to an aromatic cyclic group comprising heteroatoms consisted of 5-10 (e.g., 5, 6, 7, 8, 9, or 10) skeleton atoms.

“Fused ring” refers to a cyclic structure formed by sharing two adjacent ring atoms between rings. The fused ring may be a bicyclic ring, a tricyclic ring, or a polycyclic ring.

“Fused ring consisted of an aromatic ring and an unsaturated aliphatic heterocycle” in the present application refers to a fused ring structure formed by sharing two adjacent ring atoms between the aromatic ring and the unsaturated aliphatic heterocycle; and “fused ring consisted of an aromatic heterocycle and an unsaturated aliphatic heterocycle” refers to a fused ring structure formed by sharing two adjacent ring atoms between the aromatic heterocycle and the unsaturated aliphatic heterocycle. “7-12 membered fused ring consisted of an aromatic heterocycle and an unsaturated aliphatic heterocycle” in the present application refers to a fused ring structure having 7-12 skeleton ring atoms formed by sharing two adjacent ring atoms between the unsaturated aliphatic heterocycle and the aromatic heterocycle. “7-12 membered fused ring consisted of an aromatic ring and an unsaturated aliphatic heterocycle” in the present application refers to a fused ring structure having 7-12 skeleton ring atoms formed by sharing two adjacent ring atoms between the unsaturated aliphatic heterocycle and the aromatic ring.

A common fused ring consisted of an aromatic ring and an unsaturated aliphatic heterocycle includes, but is not limited to:

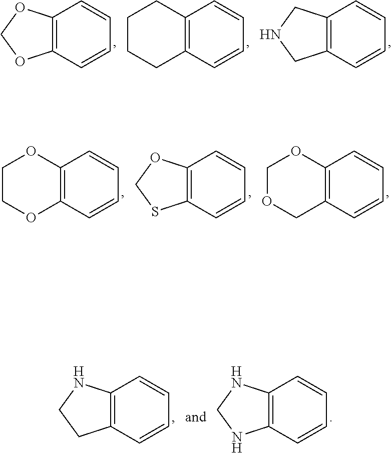

A common fused ring consisted of an aromatic heterocycle and an unsaturated aliphatic heterocycle includes, but is not limited to:

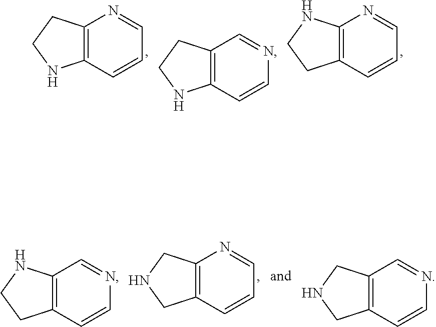

The “halogen” or “halo” refers to fluorine, chlorine, bromine or iodine.

“Haloalkyl” means that at least one hydrogen in an alkyl group is replaced by a halogen atom, and a C₁-C₆ haloalkyl, as used in this application, means a linear or branched alkyl consisting of 1-6 carbon atoms and at least one hydrogen in the alkyl is arbitrarily replaced by a halogen atom.

“Amine group” or “amine” means having a chemical structure of —NR^{U}R^{V}, wherein R^{U}, R^{V} are each independently selected from hydrogen, deuterium, tritium, alkyl, cycloalkyl.

“Imino” or “imine” means having a chemical structure of =NR^{W}, wherein R^{W} is selected from hydrogen, deuterium, tritium, alkyl, cycloalkyl.

“Amide” or “amido” means having a chemical structure of —C(O)NR^{X}R^{Y} or —NR^{X}C(O)R^{Y}, wherein R^{X}, R^{Y} are each independently selected from hydrogen, deuterium, tritium, alkyl, cycloalkyl, and common amido includes, but is not limited to —CONH₂, —CONHCH₃, —CON(CH₃)₂, —NHCOH, —NHCOCH₃, —N(CH₃)COCH₃.

“Ester group” means having a chemical structure of a formula of —COOR⁰, wherein R⁰ is selected from alkyl, cycloalkyl, heterocycloalkyl, aryl, heteroaryl.

“Substituted” means that one or more hydrogen atoms in a group are substituted independently by a corresponding number of substituents. It goes without saying that, the substituents are only in their possible chemical positions, and those of skills in the art are able to determine (either experimentally or theoretically) possible or impossible substitutions without undue effort. For example, an amino or hydroxyl with a free hydrogen may be unstable when binds to a carbon atom with an unsaturated (e.g. olefinic) bond. Each is independently selected from alkyl, cycloalkyl, aryl, heteroaryl, heterocycloalkyl, hydroxy, alkoxy, alkylthio, aryloxy, nitro, acyl, halogen, haloalkyl, amino, and the like.

“Inhibitor” refers to a substance reducing the activity of an enzyme.

“Optional” or “optionally” means that the event or circumstance subsequently described may, but not necessarily, occur, and the description includes a situation when the event or circumstance does or does not occur. For example, “optionally substituted” includes substituted or unsubstituted, e.g., “a heterocyclic group optionally substituted by an alkyl” means that the alkyl may, but not necessarily, be present, and the description includes a situation in which the heterocyclic group is substituted by the alkyl and a situation in which the heterocyclic group is not substituted by the alkyl.

“Pharmaceutical composition” indicates a mixture comprising one or more of the compounds described herein, or a physiologically/pharmaceutically acceptable salt or prodrug thereof, with other chemical components, as well as other components such as physiologically/pharmaceutically acceptable carriers and vehicles. The pharmaceutical composition is intended to facilitate administration to an organism and facilitate absorption of an active ingredient to exert the biological activity.

“Pharmaceutically acceptable” refers to those compounds, materials, compositions and/or dosage forms that are suitable for use in contact with human and animal tissues within a range of sound medical judgment, without undue toxicity, irritation, allergic response or other problems or complications, commensurate with a reasonable benefit/risk ratio.

As pharmaceutically acceptable salts, mention may be made of, e.g., metal salts, ammonium salts, salts formed with organic bases, salts formed with inorganic acids, salts formed with organic acids, salts formed with basic or acidic amino acids, and the like.

“Tautomer” or “tautomeric form” refers to structural isomers of different energies that can be interconverted through low-energy barriers. For example, proton tautomer (also known as proton transfer tautomer) includes tautomerism via proton migration, such as keto-enol and imine-enamine isomerization. Specific example of proton tautomer is imidazole moiety, wherein the proton can migrate between the two ring nitrogens. Valence tautomers include interconversion by recombination of some bonding electrons. Non-limiting examples of tautomers include, but are not limited to,

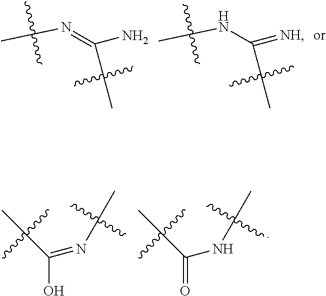 [SMILES: */N=C(\*)N ; *NC(*)=N ; */N=C(\*)O ; *NC(*)=O]

“Stereoisomer” refers to an isomer resulting from a different spatial arrangement of atoms in a molecule.

“Enantiomer” refers to isomerism caused by different spatial configurations of the atoms of compounds having the same molecular formula and functional groups, and said compounds form stereoisomers that are mirror images of each other and cannot overlap.

“Diastereoisomer” refers to isomerism caused by different spatial configurations of the atoms of compounds having the same molecular formula and functional groups, and said compounds are stereoisomers that do not exhibit a physical or mirror image relationship with each other.

Unless otherwise indicated, the terms “comprise, comprises and comprising” or their equivalents (contain, contains, containing, include, includes, including) used herein are open-ended mode expressions, and mean that other unspecified elements, components and steps may also be covered, in addition to the elements, components and steps listed.

Unless otherwise indicated, all numbers used herein to denote amounts of ingredients, measurements, or reaction conditions should be understood to be modified in all cases by the term “about”. When associated with a percentage, the term “about” may indicate, for example, ±1%, preferably ±0.5%, more preferably ±0.1%.

Unless otherwise specified clearly in the context, singular terms herein cover plural referents, and vice versa. Similarly, unless otherwise specified clearly in the context, the word “or” herein is intended to include “and”.

Apparently, according to the above contents of the application, in accordance with the ordinary technical knowledge and means in the field, under the premise of not departing from the above basic technical concepts of the application, a variety of other forms of modifications, substitutions or changes can also be made.

The abbreviations in the application have the meanings indicated below:

| | |
|---|---|
| 0-rt represents a reaction temperature from 0° C. to room | DMF represents N,N-dimethylformamide; |
| temperature; | |
| Pd(dppf)Cl₂ represents [1,1′- | THF represents tetrahydrofuran; |
| bis(diphenylphosphino)ferrocene]dichloropalladium; | |
| MeOH represents methanol; | KOH represents potassium hydroxide; |
| HATU represents 2-(7-azabenzotriazol-1-yl)-N,N,N′,N′- | DIPEA or DIEA represents N,N- |
| tetramethyluronium hexafluorophosphate; | diisopropylethylamine; |
| K₂CO₃ represents potassium carbonate; | Na₂CO₃ represents sodium carbonate; |
| tBuONO represents tert-butyl nitrite; | MeONa represents sodium methoxide; |
| I₂ represents iodine; | DCM represents dichloromethane; |
| PhN(Tf)₂ represents N- | NaSMe represents sodium thiomethoxide; |
| phenylbis(trifluoromethanesulphonimide); | |
| K₄Fe(CN)₆•3H₂O represents potassium | CuI represents cuprous iodide; |
| hexacyanoferrate(II) trihydrate; | |
| ACN represents acetonitrile; | Na₂CO₃ represents sodium carbonate; |
| dioxane/H₂O represents dioxane aqueous solution; | KOAc represents potassium acetate; |
| XphosPdG3 represents methanesulfonato(2- | Xphos represents 2-dicyclohexylphosphino- |
| dicyclohexylphosphino-2′,4′,6′-tri-isopropyl-1,1′- | 2,4,6-triisopropylbiphenyl. |
| biphenyl)(2′-amino-1,1′-biphenyl-2-yl)palladium (II); | |

### EMBODIMENTS OF THE INVENTION

The methods of synthesizing the compounds and intermediates of the present application are described below by way of example. The following examples are only intended to serve as examples of the present application, and should not be taken as a limitation to the scope of the present application. Unless otherwise indicated, the raw materials and reagents involved in the present application are all available commercially, and the specific source does not affect the implementation of the technical solution of the present application.

### Preparation Example 1: Preparation of (7-cyclopropyl-3-methoxythieno[2,3-b]pyrazin-6-yl)(piperidin-1-yl)methanone

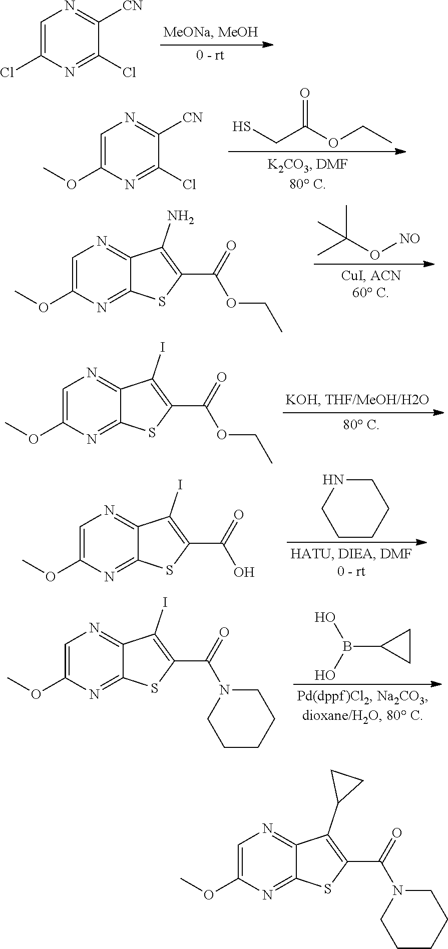

### Step 1: Preparation of 3-chloro-5-methoxypyrazine-2-carbonitrile

3,5-dichloropyrazine-2-carbonitrile (20.0 g) was weighed and dissolved in methanol (100 mL), into which sodium methoxide (6.8 g) was added at 0° C. to react at 0° C. for 3 h, then heated to room temperature, and stirred for 1 h. When TLC showed that the raw materials were completely consumed, it was concentrated under reduced pressure, quenched by adding water, extracted with ethyl acetate for twice, dried over anhydrous sodium sulfate, and purified by a silica gel column chromatography to obtain the title compound (9.0 g). MS (ESI) m/z (M+H)⁺=170.0.

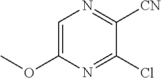

### Step 2: Preparation of ethyl 7-amino-3-methoxythieno[2,3-b]pyrazine-6-carboxylate

3-chloro-5-methoxypyrazine-2-carbonitrile (9.0 g) was weighed and dissolved in N,N-dimethylformamide (120 mL), into which potassium carbonate (16 g) and ethyl mercaptoacetate (7.0 mL) were added to react overnight at 80° C. When TLC showed that the raw materials were completely consumed, the reaction was quenched by adding water, extracted with ethyl acetate for twice, washed with saturated brine for twice, dried over anhydrous sodium sulfate, and purified by a silica gel column chromatography to obtain the title compound (8.0 g). MS (ESI) m/z (M+H)⁺=254.0.

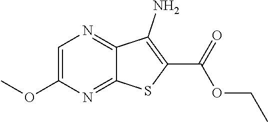 [SMILES: CCOC(=O)c1sc2nc(OC)cnc2c1N]

### Step 3: Preparation of ethyl 7-iodo-3-methoxythieno[2,3-b]pyrazine-6-carboxylate

Ethyl 7-amino-3-methoxythieno[2,3-b]pyrazine-6-carboxylate (9.0 g) and cuprous iodide (12 g) were weighed and dissolved in 120 mL of acetonitrile, into which tert-butyl nitrite (7.58 mL) was added dropwise at 60° C. to react at 60° C. for 6 h. When TLC monitored that the reaction was completed, it was diluted with EA, filtered with diatomite, quenched by adding water, extracted with ethyl acetate for twice, dried over anhydrous sodium sulfate, and purified by a silica gel column chromatography to obtain the title compound (6.47 g). MS (ESI) m/z (M+H)⁺=365.0.

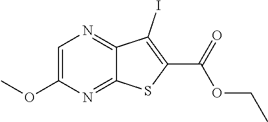 [SMILES: CCOC(=O)c1sc2nc(OC)cnc2c1I]

### Step 4: Preparation of 7-iodo-3-methoxythieno[2,3-b]pyrazine-6-carboxylic acid

Ethyl 7-iodo-3-methoxythieno[2,3-b]pyrazine-6-carboxylate (6.47 g) was weighed and dissolved in a mixed solvent of tetrahydrofuran (30 mL), methanol (10 mL) and water (10 mL), into which potassium hydroxide (3.0 g) was added to react at 80° C. for 3 h. When LCMS showed that the raw materials were completely consumed, the reaction was quenched by adding water, adjusted to an acidic pH value with 2 M hydrochloric acid, extracted with ethyl acetate for three times, dried over anhydrous sodium sulfate, and concentrated under reduced pressure to obtain the title compound (3.6 g). MS (ESI) m/z (M+H)⁺=336.9.

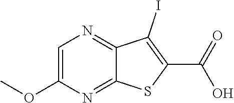

### Step 5: Preparation of (7-iodo-3-methoxythieno[2,3-b]pyrazin-6-yl)(piperidin-1-yl)methanone

7-iodo-3-methoxythieno[2,3-b]pyrazine-6-carboxylic acid (3.6 g) was weighed and dissolved in N,N-dimethylformamide (50 mL), into which N,N-diisopropylethylamine (3.72 mL), hexahydropyridine (1.65 mL) and 2-(7-azabenzotriazol-1-yl)-N,N,N′,N′-tetramethyluronium hexafluorophosphate (6.1 g) were sequentially added at 0° C., and then heated to room temperature to react for 3 h. When TLC showed that the raw materials were completely consumed, the reaction was quenched by adding water, extracted with ethyl acetate for twice, washed with saturated brine for twice, dried over anhydrous sodium sulfate, and purified by a silica gel column chromatography to obtain the title compound (2.5 g). MS (ESI) m/z (M+H)⁺=404.0.

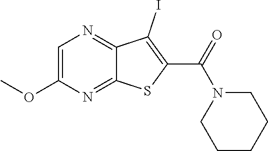 [SMILES: COc1cnc2c(I)c(C(=O)N3CCCCC3)sc2n1]

### Step 6: Preparation of (7-cyclopropyl-3-methoxythieno[2,3-b]pyrazin-6-yl)(piperidin-1-yl)methanone

(7-iodo-3-methoxythieno[2,3-b]pyrazin-6-yl)(piperidin-1-yl)methanone (1.1 g), cyclopropylboronic acid (0.46 g), sodium carbonate (8 mg) and [1,1′-bis(diphenylphosphino)ferrocene]dichloropalladium (100 mg) were weighed, into which 1,4-dioxane (15 mL) and water (5 mL) were added, and after argon replacement for three times, a reaction was carried out at 80° C. for 8 h. When LCMS monitored that the raw materials were completely reacted, the reaction was quenched by adding water, extracted with ethyl acetate for twice, dried over anhydrous sodium sulfate, and purified by a silica gel column to obtain the title compound (0.62 g). MS (ESI) m/z (M+H)⁺=318.0.

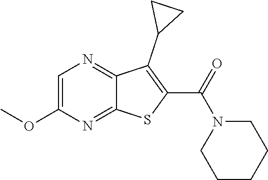 [SMILES: COc1cnc2c(C3CC3)c(C(=O)N3CCCCC3)sc2n1]

### Preparation Example 2: Preparation of 3-methoxy-6-(piperidine-1-carbonyl)thieno[2,3-b]pyrazine-7-carbonitrile

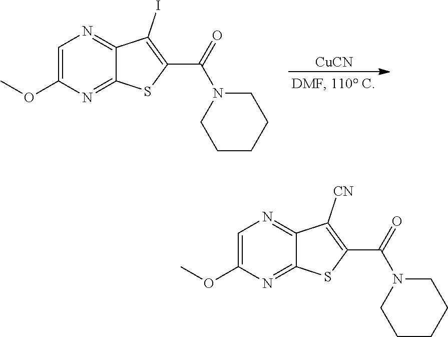 [SMILES: COc1cnc2c(I)c(C(=O)N3CCCCC3)sc2n1 ; "CuCN" ; COc1cnc2c(C#N)c(C(=O)N3CCCCC3)sc2n1]

(7-iodo-3-methoxythieno[2,3-b]pyrazin-6-yl)(piperidin-1-yl)methanone (3.6 g) obtained in Step 5 of Preparation Example 1 and cuprous cyanide (2.4 g) were weighed, into which N,N-dimethylformamide (40 mL) was added, and after argon replacement for three times, a reaction was carried out at 110° C. for 6 h. When LCMS monitored that the raw materials were completely reacted, evaporation was performed under reduced pressure to remove the solvent, and a saturated sodium sulfide solution was added thereto, stirred at room temperature for 30 min, and filtered. Filter cake was washed with ethyl acetate. Filtrates were combined, concentrated, and purified by a silica gel column to obtain the title compound (2.2 g). MS (ESI) m/z (M+H)⁺=303.1.

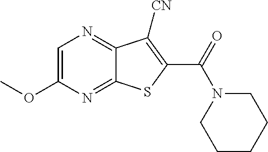 [SMILES: COc1cnc2c(C#N)c(C(=O)N3CCCCC3)sc2n1]

### Preparation Example 3: Preparation of (3-methoxy-7-(trifluoromethyl)thieno[2,3-b]pyrazin-6-yl)(piperidin-1-yl)methanone

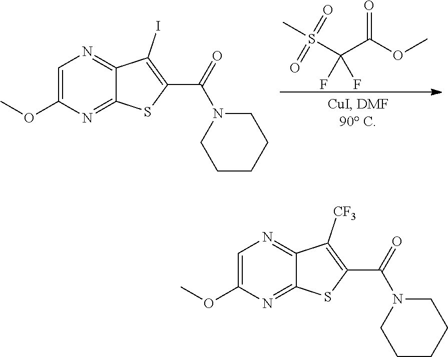

(7-iodo-3-methoxythieno[2,3-b]pyrazin-6-yl)(piperidin-1-yl)methanone (1.0 g) was weighed and dissolved in N,N-dimethylformamide (10 mL), into which methyl 2,2-difluoro-2-(fluorosulfonyl)acetate (0.95 mL) and cuprous iodide (95 mg) were added, and after argon replacement for three times, a reaction was carried out at 90° C. for 4 h. The reaction was quenched by adding water, extracted with ethyl acetate for twice, dried over anhydrous sodium sulfate, and purified by a silica gel column chromatography to obtain the title compound (845 mg). MS (ESI) m/z (M+H)⁺=346.0.

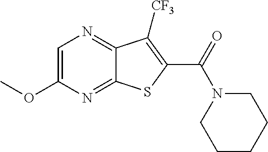 [SMILES: COc1cnc2c(C(F)(F)F)c(C(=O)N3CCCCC3)sc2n1]

### Example 1: Preparation of (7-cyclopropyl-3-phenylthieno[2,3-b]pyrazin-6-yl)(piperidin-1-yl)methanone

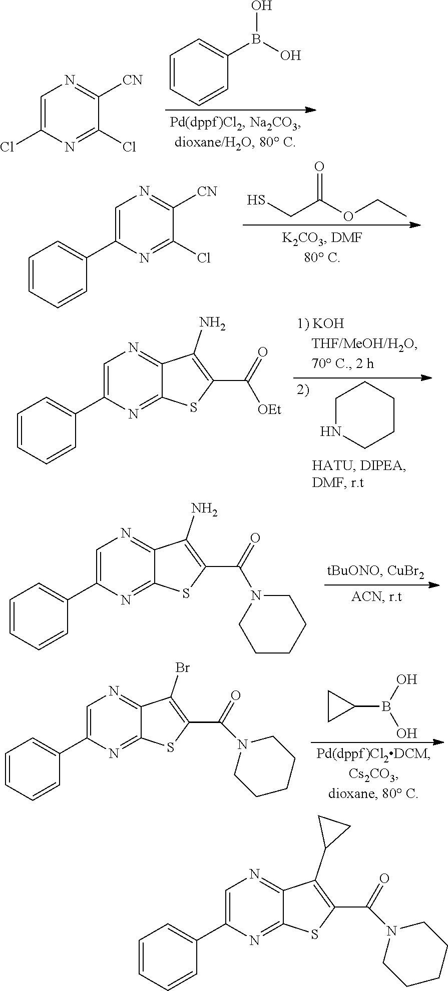

### Step 1: Preparation of 3-chloro-5-phenylpyrazine-2-carbonitrile

3,5-dichloropyrazine-2-carbonitrile (2.5 g), phenylboronic acid (1.95 g), sodium carbonate (1.84 g) and [1,1′-bis(diphenylphosphino)ferrocene]dichloropalladium (0.53 g) were weighed and dissolved in a mixed solvent of 1,4-dioxane (20 mL) and water (5 mL), and after nitrogen replacement for three times, a reaction was carried out at 80° C. for 2 h. TLC monitored that the raw materials were completely reacted. After being cooled to room temperature, it was filtered. Filtrate was added with water and extracted with ethyl acetate. Organic phases were combined, dried over anhydrous sodium sulfate, and evaporated under reduced pressure to remove the solvent. A residue was purified by a silica gel column to obtain a crude product of the title compound (3.1 g). MS (ESI) m/z (M+H)⁺=216.0.

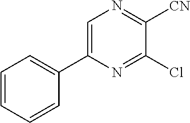 [SMILES: N#Cc1ncc(-c2ccccc2)nc1Cl]

### Step 2: Preparation of ethyl 7-amino-3-phenylthieno[2,3-b]pyrazine-6-carboxylate

3-chloro-5-phenylpyrazine-2-carbonitrile (80 mg) was weighed and dissolved in N,N-dimethylformamide (2 mL), into which potassium carbonate (120 mg) and ethyl mercaptoacetate (54 μL) were added to react overnight at 80° C. LC-MS monitored that the reaction was completed. It was cooled to room temperature, quenched by adding water, and extracted with ethyl acetate for three times. Organic phases were combined, dried over anhydrous sodium sulfate, concentrated under reduced pressure, and purified by a silica gel column to obtain the title compound (90 mg). MS (ESI) m/z (M+H)⁺=300.1.

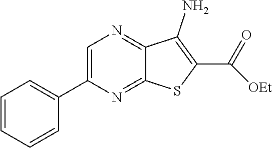 [SMILES: CCOC(=O)c1sc2nc(-c3ccccc3)cnc2c1N]

### Step 3: Preparation of (7-amino-3-phenylthieno[2,3-b]pyrazin-6-yl)(piperidin-1-yl)methanone

(1) Ethyl 7-amino-3-phenylthieno[2,3-b]pyrazine-6-carboxylate (90 mg) was weighed and dissolved in tetrahydrofuran (2 mL), methanol (0.6 mL) and water (0.6 mL), into which potassium hydroxide (51 mg) was added to react at 70° C. for 2 h. LCMS monitored that the reaction was completed. It was cooled to room temperature, water was added thereto, and extraction was performed with ethyl acetate for three times to discard an organic phase. An aqueous phase was adjusted to pH 2 with 2 M diluted hydrochloric acid, and then extracted with ethyl acetate. Organic phases were combined, dried over anhydrous sodium sulfate, and evaporated under reduced pressure to remove the solvent to obtain a crude product of 7-amino-3-phenylthieno[2,3-b]pyrazine-6-carboxylic acid.

(2) The crude product was dissolved in N,N-dimethylformamide (2 mL), into which diisopropylethylamine (100 μL), piperidine (37 μL) and 2-(7-azabenzotriazol-1-yl)-N,N,N′,N′-tetramethyluronium hexafluorophosphate (170 mg) were sequentially added under an ice water bath, then transferred to room temperature, and stirred for 2 h. LCMS monitored that the reaction was completed. The reaction was quenched by adding water, and extracted with ethyl acetate for three times. Organic phases were combined, dried over anhydrous sodium sulfate, and concentrated under reduced pressure. A residue was purified by a reverse phase preparative chromatography to obtain the title compound (9.5 mg). MS (ESI) m/z (M+H)⁺=339.1.

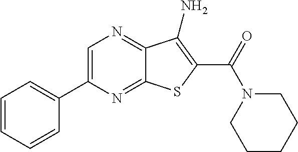 [SMILES: Nc1c(C(=O)N2CCCCC2)sc2nc(-c3ccccc3)cnc12]

### Step 4: Preparation of (7-bromo-3-phenylthieno[2,3-b]pyrazin-6-yl)(piperidin-1-yl)methanone

(7-amino-3-phenylthieno[2,3-b]pyrazin-6-yl)(piperidin-1-yl)methanone (130 mg) was weighed and dissolved in dry acetonitrile (3 mL), into which copper bromide (100 mg) was added under an ice water bath with the protection of nitrogen, and then tert-butyl nitrite (57 μL) was added dropwise, and transferred to room temperature to react for 1 h. TLC monitored that the reaction was completed. The reaction was quenched by adding a saturated sodium bicarbonate solution, and filtered to remove insoluble substances. Filtrate was extracted with ethyl acetate. Organic phases were combined and dried over anhydrous sodium sulfate. Evaporation was performed under reduced pressure to remove the solvent, purification was performed by a silica gel column to obtain a crude product, and then the crude product was separated by Prep-HPLC to obtain the title compound (30 mg). MS (ESI) m/z (M+H)⁺=402.0.

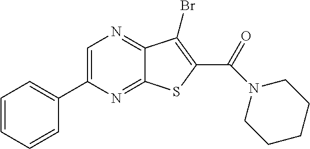 [SMILES: O=C(c1sc2nc(-c3ccccc3)cnc2c1Br)N1CCCCC1]

### Step 5: Preparation of (7-cyclopropyl-3-phenylthieno[2,3-b]pyrazin-6-yl)(piperidin-1-yl)methanone

(7-bromo-3-phenylthieno[2,3-b]pyrazin-6-yl)(piperidin-1-yl)methanone (30 mg), cyclopropyl boronic acid (8 mg), sodium carbonate (16 mg) and [1,1′-bis(diphenylphosphino)ferrocene]dichloropalladium dichloromethane complex (6.5 mg) were weighed and dissolved in 1,4-dioxane (2 mL) and water (0.6 mL), and after nitrogen replacement was performed for three times, a reaction was carried out at 100° C. for 3 h. TLC monitored that the raw materials were completely reacted. It was cooled to room temperature, and then filtered. Filtrate was added with water and extracted with ethyl acetate. Organic phases were combined, dried over anhydrous sodium sulfate, evaporated under reduced pressure to remove the solvent, and purified by reverse phase preparative chromatography to obtain the title compound (2.56 mg).

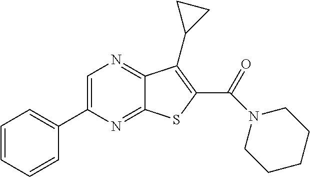 [SMILES: O=C(c1sc2nc(-c3ccccc3)cnc2c1C1CC1)N1CCCCC1]

MS (ESI) m/z (M+H)⁺=364.1. ¹H NMR (400 MHz, DMSO-d₆) δ 9.37 (s, 1H), 8.23-8.20 (m, 2H), 7.59-7.51 (m, 3H), 3.66-3.45 (m, 4H), 2.14-2.09 (m, 1H), 1.64-1.56 (m, 6H), 1.41-1.37 (m, 2H), 1.02-0.98 (m, 2H).

### Example 2: Preparation of (7-cyclopropyl-3-(2-methylpyridin-4-yl)thieno[2,3-b]pyrazin-6-yl)(piperidin-1-yl)methanone

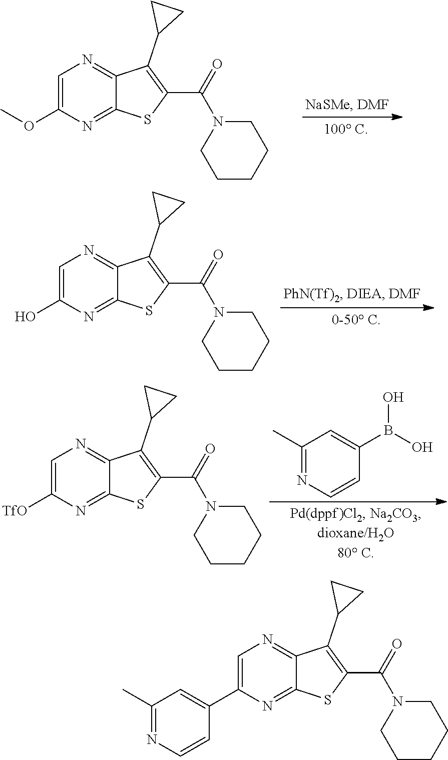

### Step 1: Preparation of (7-cyclopropyl-3-hydroxythieno[2,3-b]pyrazin-6-yl)(piperidin-1-yl)methanone

(7-cyclopropyl-3-methoxythieno[2,3-b]pyrazin-6-yl)(piperidin-1-yl)methanone (0.62 g) was weighed and dissolved in N,N-dimethylformamide (3 mL), into which sodium methyl mercaptide (20% aqueous solution, 1.4 mL) was added to react overnight at 100° C. The reaction was quenched by adding water, adjusted to an acidic pH value with 2 M hydrochloric acid, extracted with ethyl acetate for twice, washed with saturated brine for twice, dried over anhydrous sodium sulfate, and purified by a silica gel column chromatography to obtain a crude product of the title compound (0.6 g). MS (ESI) m/z (M+H)⁺=304.0.

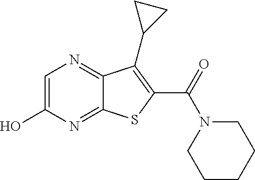 [SMILES: O=C(c1sc2nc(O)cnc2c1C1CC1)N1CCCCC1]

### Step 2: Preparation of 7-cyclopropyl-6-(piperidine-1-carbonyl)thieno[2,3-b]pyrazin-3-yltrifluoromethanesulfonate

(7-cyclopropyl-3-hydroxythieno[2,3-b]pyrazin-6-yl)(piperidin-1-yl)methanone (600 mg) was weighed and dissolved in dichloromethane (12 mL), into which N,N-diisopropylethylamine (0.62 mL) and N-phenylbis(trifluoromethanesulphonimide) (1.1 g) were added to react overnight at 50° C. When TLC and LCMS monitored that the raw materials were completely reacted, the reaction was quenched by adding water, extracted with dichloromethane for twice, dried over anhydrous sodium sulfate, and purified by a silica gel column chromatography to obtain the title compound (0.8 g). MS (ESI) m/z (M+H)⁺=436.0.

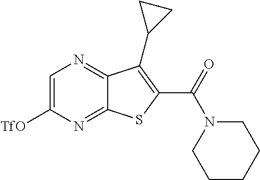 [SMILES: O=C(c1sc2nc(OS(=O)(=O)C(F)(F)F)cnc2c1C1CC1)N1CCCCC1]

### Step 3: Preparation of (7-cyclopropyl-3-(2-methylpyridin-4-yl)thieno[2,3-b]pyrazin-6-yl)(piperidin-1-yl)methanone

7-cyclopropyl-6-(piperidine-1-carbonyl)thieno[2,3-b]pyrazin-3-yltrifluoromethanesulfonate (30 mg), (2-methylpyridin-4-yl)boronic acid (14 mg), sodium carbonate (12 mg) and [1,1′-bis(diphenylphosphino)ferrocene]dichloropalladium (2 mg) were weighed, into which 1,4-dioxane (1 mL) and water (0.3 mL) were added, and after argon replacement for three times, a reaction was carried out at 80° C. for 1.5 h. When LCMS monitored that the raw materials were completely reacted, the reaction was quenched by adding water, extracted with ethyl acetate for twice, dried over anhydrous sodium sulfate, and separated by reverse phase preparative column to obtain the title compound (9.05 mg).

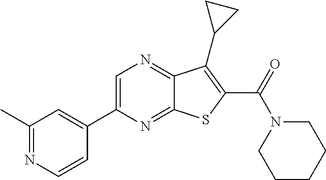 [SMILES: Cc1cc(-c2cnc3c(C4CC4)c(C(=O)N4CCCCC4)sc3n2)ccn1]

MS (ESI) m/z (M+H)⁺=379.0. ¹H NMR (400 MHz, DMSO-d₆) δ 9.46 (s, 1H), 8.64-8.63 (d, J=5.2 Hz, 1H), 8.07 (s, 1H), 7.99-7.97 (d, J=5.9 Hz, 1H), 3.67 (m, 2H), 3.45 (m, 2H), 2.59 (s, 3H), 2.15-2.10 (i, 1H), 1.67-1.55 (m, 6H), 1.41-1.37 (dt, J=5.9, 3.0 Hz, 2H), 1.03-0.99 (dt, J=8.6, 3.1 Hz, 2H).

A series of compounds were prepared from corresponding commercial reagents and the products in the foregoing Preparation Examples and Examples as raw materials, by using a preparation method similar to that of the foregoing example, and structures and characterization data of the compounds are shown in Table 1.

TABLE 1

| | | Refer- | |
|---|---|---|---|
| | | ence | |
| | | example | |
| | | of | |
| Ex- | | prepara- | |
| am- | | tion | |
| ple | Structure | method | MS (M + H)⁺ & ¹H NMR |
| 3 | 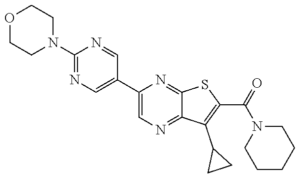 [SMILES: O=C(c1sc2nc(-c3cnc(N4CCOCC4)nc3)cnc2c1C1CC1)N1CCCCC1] | Example 2 | MS (ESI) m/z (M + H)⁺ = 451.1, ¹H NMR (400 MHz, DMSO-d₆) δ 9.30 (s, 1H), 9.18 (s, 2H), 3.85-3.82 (t, J = 4.7 Hz, 4H), 3.71-3.68 (t, J = 4.7 Hz, 4H), 3.45 (m, 4H), 2.13-2.06 (tt, J = 8.5, 5.3 Hz, 1H), 1.65-1.56 (m, 6H), 1.41-1.37 (dt, J = 5.9, 2.9 Hz, 2H), 1.01-0.97 (dt, J = 8.6, 3.2 Hz, 2H) |
| 4 | 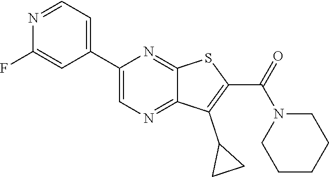 [SMILES: O=C(c1sc2nc(-c3ccnc(F)c3)cnc2c1C1CC1)N1CCCCC1] | Example 2 | MS (ESI) m/z (M + H)⁺ = 383.1, ¹H NMR (400 MHz, DMSO-d₆) δ 9.53 (s, 1H), 8.45- 8.44 (d, J = 5.3 Hz, 1H), 8.18-8.16 (dt, J = 5.4, 1.7 Hz, 1H), 7.98 (s, 1H), 3.67-3.44 (m, 4H), 2.13-2.10 (tt, J = 8.6, 5.3 Hz, 1H), 1.66-1.55 (m, 6H), 1.41- 1.37 (dt, J = 6.0, 3.0 Hz, 2H), 1.04-0.99 (m, 2H) |
| 5 | 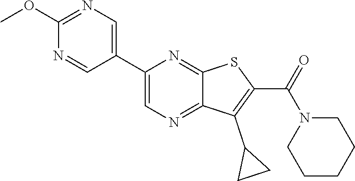 [SMILES: COc1ncc(-c2cnc3c(C4CC4)c(C(=O)N4CCCCC4)sc3n2)cn1] | Example 2 | MS (ESI) m/z (M + H)⁺ = 396.1, ¹H NMR (400 MHz, DMSO-d₆) δ 9.39 (s, 1H), 9.38 (s, 2H), 4.02 (s, 3H), 3.66-3.44 (m, 4H), 2.12-2.08 (m, 1H), 1.67-1.57 (m, 6H), 1.41-1.37 (dt, J = 6.0, 3.0 Hz, 2H), 1.04-0.98 (m, 2H) |
| 6 | 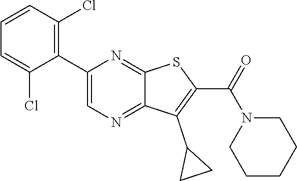 [SMILES: O=C(c1sc2nc(-c3c(Cl)cccc3Cl)cnc2c1C1CC1)N1CCCCC1] | Example 2 | MS (ESI) m/z (M + H)⁺ = 433.1, ¹H NMR (400 MHz, DMSO-d₆) δ 8.89 (s, 1H), 7.71- 7.69 (m, 2H), 7.63-7.59 (m, 1H), 3.68-3.47 (m, 4H), 2.18-2.11 (tt, J = 8.7, 5.3 Hz, 1H), 1.66-1.57 (m, 6H), 1.39-1.35 (dt, J = 6.0, 3.0 Hz, 2H), 1.04-0.99 (dt, J = 8.7, 3.1 Hz, 2H) |
| 7 | 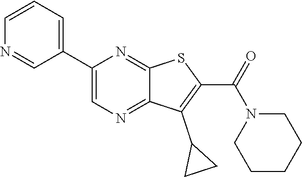 [SMILES: O=C(c1sc2nc(-c3cccnc3)cnc2c1C1CC1)N1CCCCC1] | Example 2 | MS (ESI) m/z (M + H)⁺ = 365.1, ¹H NMR (400 MHz, DMSO-d6) δ 9.47-9.36 (m, 2H), 8.74-8.71 (d, J = 5.0 Hz, 1H), 8.57-8.55 (d, J = 8.0 Hz, 1H), 7.62-7.58 (dd, J = 8.0, 4.8 Hz, 1H), 3.77- 3.42 (m, 4H), 2.12 (dq, J = 11.2, 5.3 Hz, 1H), 1.67- 1.57 (m, 6H), 1.40-1.38 (m, 2H), 1.02-0.99 (dd, J = 8.4, 2.6 Hz, 2H) |
| 8 | 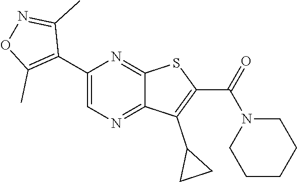 [SMILES: Cc1noc(C)c1-c1cnc2c(C3CC3)c(C(=O)N3CCCCC3)sc2n1] | Example 2 | MS (ESI) m/z (M + H)⁺ = 383.2, ¹H NMR (400 MHz, DMSO-d₆) 8.93 (s, 1H), 3.66- 3.44 (m, 4H), 2.64 (s, 3H), 2.43 (s, 3H), 2.13-2.09 (m, 1H), 1.65-1.56 (m, 6H), 1.40-1.36 (td, J = 6.0, 3.8 Hz, 2H), 1.02-0.98 (m, 2H) |
| 9 | 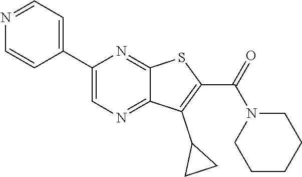 [SMILES: O=C(c1sc2nc(-c3ccncc3)cnc2c1C1CC1)N1CCCCC1] | Example 2 | MS (ESI) m/z (M + H)⁺ = 365.1, ¹H NMR (400 MHz, DMSO-d₆) δ 9.50 (s, 1H), 8.79- 8.77 (d, J = 5.7 Hz, 2H), 8.20-8.18 (m, 2H), 3.67- 3.45 (m, 4H), 2.16-2.10 (m, 1H), 1.67-1.53 (m, 6H), 1.41-1.37 (dt, J = 5.9, 2.9 Hz, 2H), 1.04-0.99 (dt, J = 8.7, 3.1 Hz, 2H) |
| 10 | 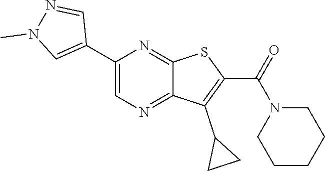 [SMILES: Cn1cc(-c2cnc3c(C4CC4)c(C(=O)N4CCCCC4)sc3n2)cn1] | Example 2 | MS (ESI) m/z (M + H)⁺ = 368.1, ¹H NMR (400 MHz, DMSO-d₆) δ 9.06 (s, 1H), 8.52 (s, 1H), 8.19 (s, 1H), 3.93 (s, 3H), 3.63-3.45 (m, 4H), 2.11-2.04 (m, 1H), 1.66-1.56 (m, 6H), 1.38-1.36 (dd, J = 5.5, 2.2 Hz, 2H), 0.99-0.96 (m, 2H) |
| 15 | 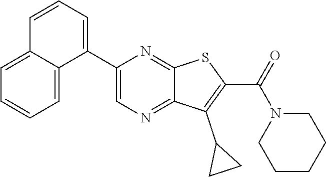 [SMILES: O=C(c1sc2nc(-c3cccc4ccccc34)cnc2c1C1CC1)N1CCCCC1] | Example 2 | MS (ESI) m/z (M + H)⁺ = 414.2, ¹H NMR (400 MHz, DMSO-d₆) δ 9.03 (s, 1H), 8.13- 8.11 (d, J = 8.1 Hz, 2H), 8.08-8.06 (dd, J = 7.8, 1.7 Hz, 1H), 7.80-7.78 (dd, J = 7.1, 1.2 Hz, 1H), 7.70- 7.67 (t, J = 7.6 Hz, 1H), 7.63-7.56 (m, 2H), 3.68-3.49 (m, 4H), 2.19-2.12 (m, 1H), 1.68-1.58 (m, 6H), 1.45-1.41 (m, 2H), 1.05-1.01 (dt, J = 8.6, 3.1 Hz, 2H) |
| 11 | 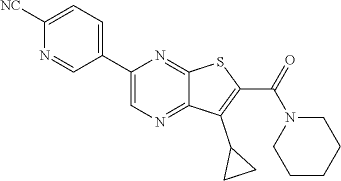 [SMILES: N#Cc1ccc(-c2cnc3c(C4CC4)c(C(=O)N4CCCCC4)sc3n2)cn1] | Example 2 | MS (ESI) m/z (M + H)⁺ = 390.1, ¹H NMR (400 MHz, DMSO-d₆) δ 9.57-9.56 (d, J = 2.2 Hz, 1H), 9.54 (s, 1H), 8.84-8.81 (dd, J = 8.2, 2.3 Hz, 1H), 8.27-8.25 (d, J = 8.2 Hz, 1H), 3.67-3.44 (d, J = 92.6 Hz, 4H), 2.16-2.10 (m, 1H), 1.64-1.56 (d, J = 33.3 Hz, 6H), 1.41-1.37 (dt, J = 6.0, 3.0 Hz, 2H), 1.04-1.00 (m, 2H) |
| 16 | 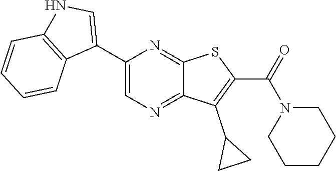 [SMILES: O=C(c1sc2nc(-c3c[nH]c4ccccc34)cnc2c1C1CC1)N1CCCCC1] | Example 2 | MS (ESI) m/z (M + H)⁺ = 403.2, ¹H NMR (400 MHz, DMSO-d₆) δ 11.87 (s, 1H), 9.27 (s, 1H), 8.51-8.49 (d, J = 7.1 Hz, 1H), 8.45 (d, J = 2.8 Hz, 1H), 7.51-7.49 (d, J = 7.1 Hz, 1H), 7.25-7.18 (p, J = 7.2 Hz, 2H), 3.56 (m, 4H), 2.11-2.06 (tt, J = 8.6, 5.3 Hz, 1H), 1.64-1.62 (m, 6H), 1.42-1.40 (dd, J = 5.5, 2.2 Hz, 2H), 1.00-0.97 (dd, J = 8.6, 2.4 Hz, 2H) |
| 17 | 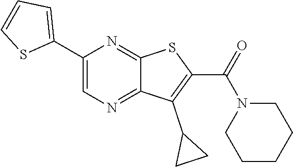 [SMILES: O=C(c1sc2nc(-c3cccs3)cnc2c1C1CC1)N1CCCCC1] | Example 2 | MS (ESI) m/z (M + H)⁺ = 370.1, ¹H NMR (400 MHz, DMSO-d₆) δ 9.34 (s, 1H), 8.10- 8.08 (dd, J = 3.8, 1.1 Hz, 1H), 7.83-7.81 (dd, J = 5.0, 1.1 Hz, 1H), 7.28-7.26 (dd, J = 5.1, 3.7 Hz, 1H), 3.65-3.45 (m, 4H), 2.12-2.05 (m, 1H), 1.64-1.56 (m, 6H), 1.40-1.36 (m, 2H), 1.01-0.97 (m, 2H) |
| 18 | 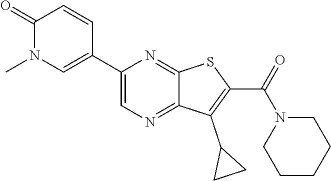 [SMILES: Cn1cc(-c2cnc3c(C4CC4)c(C(=O)N4CCCCC4)sc3n2)ccc1=O] | Example 2 | MS (ESI) m/z (M + H)⁺ = 395.2, ¹H NMR (400 MHz, DMSO-d₆) δ 9.19 (s, 1H), 8.76- 8.75 (d, J = 2.7 Hz, 1H), 8.27-8.24 (dd, J = 9.5, 2.7 Hz, 1H), 6.57-6.55 (d, J = 9.5 Hz, 1H), 3.64-3.43 (m, 7H), 2.12-2.05 (tt, J = 8.5, 5.2 Hz, 1H), 1.65-1.56 (m, 6H), 1.38-1.36 (dd, J = 5.5, 2.2 Hz, 2H), 1.00- 0.97 (dd, J = 8.6, 2.4 Hz, 2H) |
| 19 | 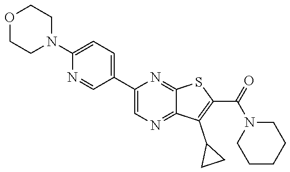 [SMILES: O=C(c1sc2nc(-c3ccc(N4CCOCC4)nc3)cnc2c1C1CC1)N1CCCCC1] | Example 2 | MS (ESI) m/z (M + H)⁺ = 450.2, ¹H NMR (400 MHz, DMSO-d₆) δ 9.29 (s, 1H), 8.99- 8.98 (d, J = 2.5 Hz, 1H), 8.35-8.32 (dd, J = 9.0, 2.6 Hz, 1H), 7.00-6.98 (d, J = 9.1 Hz, 1H), 3.73-3.43 (m, 12H), 2.12-2.05 (tt, J = 8.6, 5.3 Hz, 1H), 1.64-1.56 (m, 6H), 1.40-1.38 (dd, J = 5.5, 2.1 Hz, 2H), 0.99- 0.97 (m, 2H) |
| 20 | 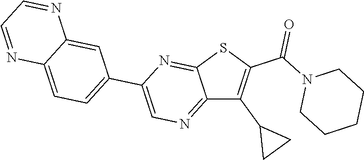 [SMILES: O=C(c1sc2nc(-c3ccc4nccnc4c3)cnc2c1C1CC1)N1CCCCC1] | Example 2 | MS (ESI) m/z (M + H)⁺ = 416.2, ¹H NMR (400 MHz, DMSO-d₆) δ 9.62 (s, 1H), 9.04- 9.00 (dd, J = 12.8, 1.9 Hz, 2H), 8.94-8.93 (d, J = 2.1 Hz, 1H), 8.70-8.68 (dd, J = 8.8, 2.0 Hz, 1H), 8.26- 8.24 (d, J = 8.8 Hz, 1H), 3.68-3.47 (m, 4H), 2.16- 2.09 (m, 1H), 1.66-1.58 (m, 6H), 1.41-1.39 (dd, J = 5.5, 2.2 Hz, 2H), 1.03-1.01 (m, 2H) |
| 21 | 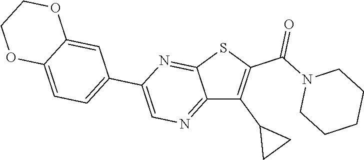 [SMILES: O=C(c1sc2nc(-c3ccc4c(c3)OCCO4)cnc2c1C1CC1)N1CCCCC1] | Example 2 | MS (ESI) m/z (M + H)⁺ = 422.2, ¹H NMR (400 MHz, DMSO-d₆) δ 9.28 (s, 1H), 7.74- 7.71 (m, 2H), 7.04-7.02 (m, 1H), 4.32 (s, 4H), 3.65- 3.44 (m, 4H), 2.12-2.06 (m, 1H), 1.66-1.56 (m, 6H), 1.40-1.36 (dt, J = 5.4, 3.1 Hz, 2H), 1.01-0.96 (m, 2H) |
| 12 | 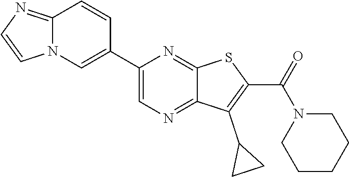 [SMILES: O=C(c1sc2nc(-c3ccc4nccn4c3)cnc2c1C1CC1)N1CCCCC1] | Example 2 | MS (ESI) m/z (M + H)⁺ = 404.1, ¹H NMR (400 MHz, DMSO-d₆) δ 9.53 (s, 1H), 9.38 (s, 1H), 8.09 (s, 1H), 8.07-8.04 (dd, J = 9.5, 1.9 Hz, 1H), 7.75-7.72 (d, J = 9.5 Hz, 1H), 7.67 (s, 1H), 3.67-3.46 (m, 4H), 2.15-2.10 (ddd, J = 8.6, 7.0, 4.3 Hz, 1H), 1.64-1.57 (m, 6H), 1.41-1.37 (dt, J = 5.4, 3.1 Hz, 2H), 1.04-0.99 (dt, J = 8.7, 3.2 Hz, 2H) |
| 13 | 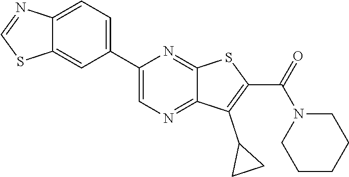 [SMILES: O=C(c1sc2nc(-c3ccc4ncsc4c3)cnc2c1C1CC1)N1CCCCC1] | Example 2 | MS (ESI) m/z (M + H)⁺ = 421.1, ¹H NMR (400 MHz, DMSO-d₆) δ 9.52 (s, 1H), 9.47 (s, 1H), 9.07 (d, J = 1.8 Hz, 1H), 8.42-8.39 (dd, J = 8.6, 1.8 Hz, 1H), 8.25-8.23 (d, J = 8.6 Hz, 1H), 3.67- 3.47 (m, 4H), 2.16-2.10 (m, 1H), 1.66-1.57 (m, 6H), 1.42-1.38 (dt, J = 5.4, 3.1 Hz, 2H), 1.04-0.99 (dt, J = 10.1, 3.2 Hz, 2H) |
| 14 | 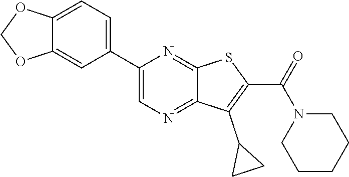 [SMILES: O=C(c1sc2nc(-c3ccc4c(c3)OCO4)cnc2c1C1CC1)N1CCCCC1] | Example 2 | MS (ESI) m/z (M + H)⁺ = 408.2, ¹H NMR (400 MHz, DMSO-d₆) δ 9.28 (s, 1H), 7.80- 7.76 (m, 2H), 7.10-7.08 (d, J = 8.1 Hz, 1H), 6.13 (s, 2H), 3.65-3.45 (m, 4H), 2.12-2.05 (dt, J = 8.6, 4.0 Hz, 1H), 1.66-1.56 (m, 6H), 1.40-1.36 (dt, J = 5.4, 3.1 Hz, 2H), 1.01-0.96 (m, 2H) |
| 22 | 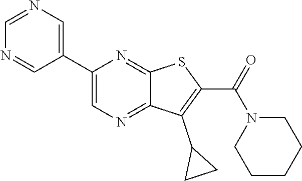 [SMILES: O=C(c1sc2nc(-c3cncnc3)cnc2c1C1CC1)N1CCCCC1] | Example 2 | MS (ESI) m/z (M + H)⁺ = 366.2, ¹H NMR (400 MHz, DMSO-d₆) δ 9.57 (s, 2H), 9.49 (s, 1H), 9.33 (s, 1H), 3.67-3.44 (m, 4H), 2.16-2.08 (ddd, J = 8.6, 7.0, 4.3 Hz, 1H), 1.66-1.57 (m, 6H), 1.41-1.37 (dt, J = 6.0, 3.0 Hz, 2H), 1.04-0.99 (m, 2H) |
| 23 | 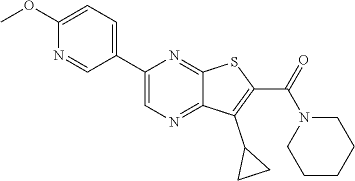 [SMILES: COc1ccc(-c2cnc3c(C4CC4)c(C(=O)N4CCCCC4)sc3n2)cn1] | Example 2 | MS (ESI) m/z (M + H)⁺ = 395.2, ¹H NMR (400 MHz, DMSO-d₆) δ 9.36 (s, 1H), 9.03 (d, J = 2.5 Hz, 1H), 8.51-8.48 (dd, J = 8.7, 2.5 Hz, 1H), 7.03-7.00 (d, J = 8.7 Hz, 1H), 3.95 (s, 3H), 3.66-3.45 (m, 4H), 2.14-2.07 (m, 1H), 1.64-1.57 (m, 6H), 1.41-1.38 (dt, J = 5.9, 3.0 Hz, 2H), 1.02-0.98 (m, 2H) |
| 24 | 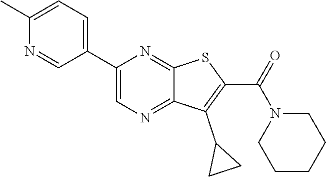 [SMILES: Cc1ccc(-c2cnc3c(C4CC4)c(C(=O)N4CCCCC4)sc3n2)cn1] | Example 2 | MS (ESI) m/z (M + H)⁺ = 379.1, ¹H NMR (400 MHz, DMSO-d₆) δ 9.39 (s, 1H), 9.26- 9.25 (d, J = 2.4 Hz, 1H), 8.47-8.44 (dd, J = 8.2, 2.4 Hz, 1H), 7.45-7.44 (d, J = 8.1 Hz, 1H), 3.66-3.45 (m, 4H), 2.56 (s, 3H), 2.14-2.07 (ddd, J = 8.6, 7.0, 4.3 Hz, 1H), 1.66-1.57 (m, 6H), 1.41-1.37 (m, 2H), 1.03-0.98 (m, 2H) |
| 25 | 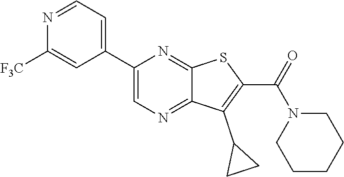 [SMILES: O=C(c1sc2nc(-c3ccnc(C(F)(F)F)c3)cnc2c1C1CC1)N1CCCCC1] | Example 2 | MS (ESI) m/z (M + H)⁺ = 433.2, ¹H NMR (400 MHz, DMSO-d₆) δ 9.62 (s, 1H), 8.98- 8.97 (d, J = 5.1 Hz, 1H), 8.63 (s, 1H), 8.54-8.53 (d, J = 5.1 Hz, 1H), 3.68-3.44 (m, 4H), 2.16-2.09 (tt, J = 8.6, 5.3 Hz, 1H), 1.65-1.55 (m, 6H), 1.41- 1.37 (dt, J = 5.4, 3.1 Hz, 2H), 1.04-1.00 (dt, J = 8.7, 3.1 Hz, 2H) |
| 26 | 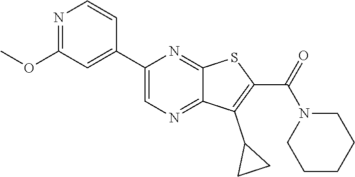 [SMILES: COc1cc(-c2cnc3c(C4CC4)c(C(=O)N4CCCCC4)sc3n2)ccn1] | Example 2 | MS (ESI) m/z (M + H)⁺ = 395.2, ¹H NMR (400 MHz, DMSO-d₆) δ 9.45 (d, J = 1.1 Hz, 1H), 8.35-8.34 (d, J = 5.4 Hz, 1H), 7.78-7.76 (d, J = 5.5 Hz, 1H), 7.60 (s, 1H), 3.93 (s, 3H), 3.67- 3.44 (m, 4H), 2.14-2.07 (ddd, J = 8.6, 7.0, 4.3 Hz, 1H), 1.66-1.56 (m, 6H), 1.40-1.36 (dt, J = 5.9, 3.0 Hz, 2H), 1.03-0.98 (m, 2H) |
| 27 | 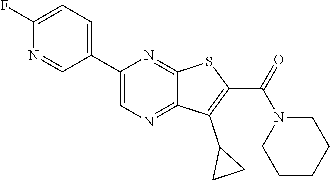 [SMILES: O=C(c1sc2nc(-c3ccc(F)nc3)cnc2c1C1CC1)N1CCCCC1] | Example 2 | MS (ESI) m/z (M + H)⁺ = 383.1, ¹H NMR (400 MHz, DMSO-d₆) δ 9.43 (s, 1H), 9.07 (d, J = 2.5 Hz, 1H), 8.79-8.74 (td, J = 8.2, 2.6 Hz, 1H), 7.43-7.40 (dd, J = 8.7, 2.7 Hz, 1H), 3.67-3.45 (m, 4H), 2.15-2.09 (ddd, J = 8.6, 7.0, 4.3 Hz, 1H), 1.67-1.57 (m, 6H), 1.41-1.37 (dt, J = 6.0, 3.0 Hz, 2H), 1.03-0.99 (m, 2H) |
| 28 | 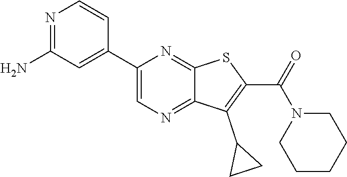 [SMILES: Nc1cc(-c2cnc3c(C4CC4)c(C(=O)N4CCCCC4)sc3n2)ccn1] | Example 2 | MS (ESI) m/z (M + H)⁺ = 380.1, ¹H NMR (400 MHz, DMSO-d₆) δ 9.29 (s, 1H), 8.09- 8.08 (d, J = 5.3 Hz, 1H), 7.26 (d, J = 1.7 Hz, 2H), 6.18 (s, 2H), 3.67-3.44 (m, 4H), 2.15-2.08 (m, 1H), 1.65-1.56 (m, 6H), 1.40-1.36 (m, 2H), 1.03-0.98 (m, 2H) |
| 29 | 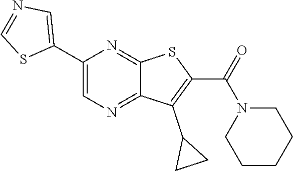 [SMILES: O=C(c1sc2nc(-c3cncs3)cnc2c1C1CC1)N1CCCCC1] | Example 2 | MS (ESI) m/z (M + H)⁺ = 371.1, ¹H NMR (400 MHz, DMSO-d₆) δ 9.42 (s, 1H), 9.29 (s, 1H), 8.87 (s, 1H), 3.66 (s, 2H), 3.44 (s, 2H), 2.13- 2.08 (m, 1H), 1.64-1.52 (m, 6H), 1.38 (dd, J = 5.4, 2.2 Hz, 2H), 1.01 (dt, J = 8.6, 3.0 Hz, 2H) |
| 30 | 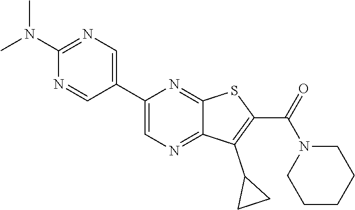 [SMILES: CN(C)c1ncc(-c2cnc3c(C4CC4)c(C(=O)N4CCCCC4)sc3n2)cn1] | Example 2 | MS (ESI) m/z (M + H)⁺ = 409.1, ¹H NMR (400 MHz, DMSO-d₆) δ 9.27 (s, 1H), 9.14 (s, 2H), 3.65 (s, 2H), 3.45 (s, 2H), 3.22 (s, 6H), 2.09 (ddd, J = 8.6, 5.8, 3.3 Hz, 1H), 1.68-1.51 (m, 6H), 1.42-1.37 (m, 2H), 1.02-0.96 (m, 2H) |
| 31 | 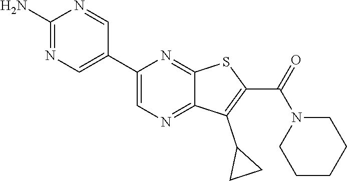 [SMILES: Nc1ncc(-c2cnc3c(C4CC4)c(C(=O)N4CCCCC4)sc3n2)cn1] | Example 2 | MS (ESI) m/z (M + H)⁺ = 381.1, ¹H NMR (400 MHz, DMSO-d₆) δ 9.26 (s, 1H), 9.05 (s, 2H), 7.25 (s, 2H), 3.65 (s, 2H), 3.45 (s, 2H), 2.09 (tt, J = 8.5, 5.3 Hz, 1H), 1.67-1.51 (m, 6H), 1.37 (dt, J = 5.9, 2.9 Hz, 2H), 1.02-0.96 (m, 2H) |
| 32 | 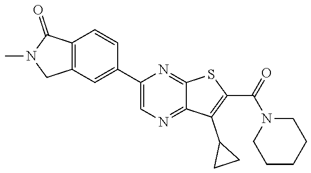 [SMILES: CN1Cc2cc(-c3cnc4c(C5CC5)c(C(=O)N5CCCCC5)sc4n3)ccc2C1=O] | Example 2 | MS (ESI) m/z (M + H)⁺ = 433.1, ¹H NMR (400 MHz, DMSO-d₆) δ 9.44 (s, 1H), 8.44 (s, 1H), 8.33 (d, J = 8.0 Hz, 1H), 7.82 (d, J = 8.0 Hz, 1H), 4.58 (s, 2H), 3.67 (s, 2H), 3.46 (s, 2H), 3.12 (s, 3H), 2.12 (d, J = 7.5 Hz, 1H), 1.64-1.57 (m, 6H), 1.39 (d, J = 5.3 Hz, 2H), 1.01 (d, J = 8.4 Hz, 2H) |

### Example 33: Preparation of (7-cyclopropyl-3-(2-methylpyridin-4-yl)thieno[2,3-b]pyrazin-6-yl)(piperidin-1-yl)methanone

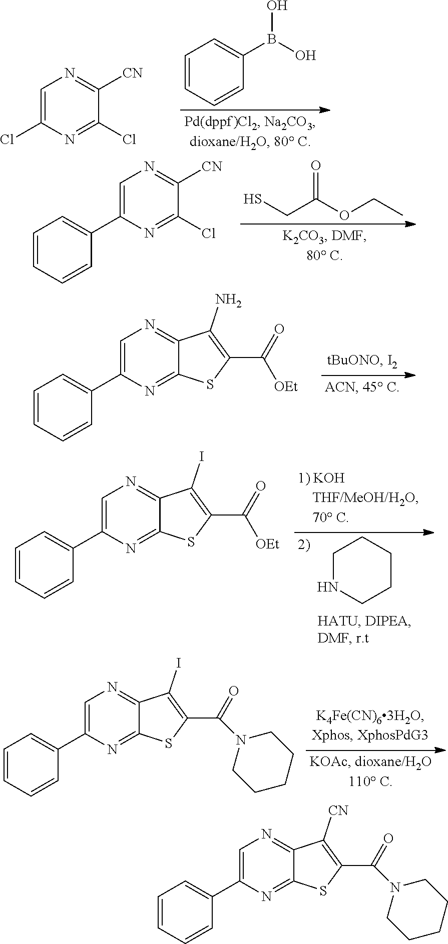

### Step 1: Preparation of 3-chloro-5-phenylpyrazine-2-carbonitrile

3,5-dichloropyrazine-2-carbonitrile (2.5 g), phenylboronic acid (1.95 g), sodium carbonate (1.84 g) and [1,1′-bis(diphenylphosphino)ferrocene]dichloropalladium (0.53 g) were weighed and dissolved in a mixed solvent of 1,4-dioxane (20 mL) and water (5 mL), and after nitrogen replacement for three times, a reaction was carried out at 80° C. for 2 h. TLC monitored that the raw materials were completely reacted. It was cooled to room temperature, and filtered. Filtrate was added with water and extracted with ethyl acetate. Organic phases were combined, dried over anhydrous sodium sulfate, and evaporated under reduced pressure to remove the solvent. A residue was purified by a silica gel column to obtain a crude product of the title compound (3.1 g). MS (ESI) m/z (M+H)⁺=216.0.

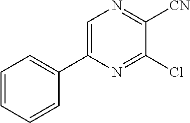 [SMILES: N#Cc1ncc(-c2ccccc2)nc1Cl]

### Step 2: Preparation of ethyl 7-amino-3-phenylthieno[2,3-b]pyrazine-6-carboxylate

3-chloro-5-phenylpyrazine-2-carbonitrile (80 mg) was weighed and dissolved in N,N-dimethylformamide (2 mL), into which potassium carbonate (120 mg) and ethyl mercaptoacetate (54 μL) were added to react overnight at 80° C. LC-MS monitored that the reaction was completed. It was cooled to room temperature, quenched by adding water, and extracted with ethyl acetate for three times. Organic phases were combined, dried over anhydrous sodium sulfate, concentrated under reduced pressure, and purified by a silica gel column to obtain the title compound (90 mg). MS (ESI) m/z (M+H)⁺=300.1.

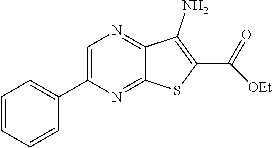 [SMILES: CCOC(=O)c1sc2nc(-c3ccccc3)cnc2c1N]

### Step 3: Preparation of ethyl 7-iodo-3-phenylthieno[2,3-b]pyrazine-6-carboxylate (BT-120-348-062)

Iodine (0.76 g) and tert-butyl nitrite (0.18 mL) were weighed and dissolved in dry acetonitrile (10 mL), into which ethyl 7-amino-3-phenylthieno[2,3-b]pyrazine-6-carboxylate (300 mg) was added under the protection of nitrogen, copper bromide (490 mg) was added thereto, and then tert-butyl nitrite (0.3 mL) was added dropwise to react at 45° C. for 2 h. TLC monitored that the reaction was completed. It was cooled to room temperature, quenched by adding a saturated sodium bisulfite solution, then adjusted to pH 8 with a saturated sodium bicarbonate solution, and extracted with ethyl acetate. Organic phases were combined and dried over anhydrous sodium sulfate. Evaporation was performed under reduced pressure to remove the solvent, and purification was performed by a silica gel column to obtain the title compound (80 mg). MS (ESI) m/z (M+H)⁺=411.1.

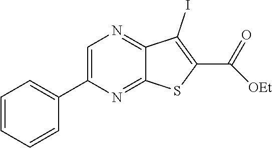 [SMILES: CCOC(=O)c1sc2nc(-c3ccccc3)cnc2c1I]

### Step 4: Preparation of (7-iodo-3-phenylthieno[2,3-b]pyrazin-6-yl)(piperidin-1-yl)methanone (BT-120-348-063)

Ethyl 7-iodo-3-phenylthieno[2,3-b]pyrazine-6-carboxylate (80 mg) was weighed and dissolved in tetrahydrofuran (1 mL), methanol (0.3 mL) and water (0.3 mL), into which potassium hydroxide (34 mg) was added to react at 70° C. for 2 h. LCMS monitored that the reaction was completed. It was cooled to room temperature, water was added thereto, and extraction was performed with ethyl acetate for three times to discard an organic phase. An aqueous phase was adjusted to pH 2 with 2 M diluted hydrochloric acid, and then extracted with ethyl acetate. Organic phases were combined, dried over anhydrous sodium sulfate, and evaporated under reduced pressure to remove the solvent to obtain a crude product. The crude product was dissolved in N,N-dimethylformamide (2 mL), into which diisopropylethylamine (65 μL), piperidine (25 μL) and 2-(7-azabenzotriazol-1-yl)-N,N,N′,N′-tetramethyluronium hexafluorophosphate (114 mg) were sequentially added under an ice water bath, then transferred to room temperature, and stirred for 2 h. LCMS monitored that a reaction was completed. The reaction was quenched by adding water, and extracted with ethyl acetate for three times. Organic phases were combined, dried over anhydrous sodium sulfate, and concentrated under reduced pressure. A residue was purified by a silica gel column to obtain the title compound (30 mg). MS (ESI) m/z (M+H)⁺=449.9.

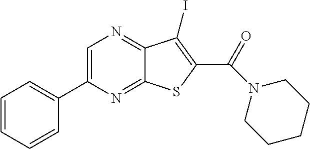 [SMILES: O=C(c1sc2nc(-c3ccccc3)cnc2c1I)N1CCCCC1]

### Step 5: Preparation of 3-phenyl-6-(piperidine-1-carbonyl)thieno[2,3-b]pyrazine-7-carbonitrile (BT-120-348-065)

(7-iodo-3-phenylthieno[2,3-b]pyrazin-6-yl)(piperidin-1-yl)methanone (30 mg) was weighed and dissolved in a mixed solvent of 1,4-dioxane (1 mL) and water (0.3 mL), into which potassium acetate (15 mg), potassium hexacyanoferrate(II) trihydrate (16 mg), 2-dicyclohexylphosphino-2,4,6-triisopropylbiphenyl (7 mg) and methanesulfonato(2-dicyclohexylphosphino-2′,4′,6′-tri-isopropyl-1,1′-biphenyl)(2′-amino-1,1′-biphenyl-2-yl)palladium (II) (6 mg) were sequentially added. After the system was replaced with nitrogen for 3 times, the system was sealed to carry out a reaction at 110° C. for 3 h. LC-MS monitored that the reaction was completed. It was cooled to room temperature, and filtered. Filter cake was washed with ethyl acetate for several times. Filtrate was added with water to separate an organic layer. An aqueous phase was extracted with ethyl acetate for three times. Organic phases were combined, dried over anhydrous sodium sulfate, and concentrated to dryness under reduced pressure. A residue was separated by Prep-HPLC to obtain the title compound (2.18 mg).

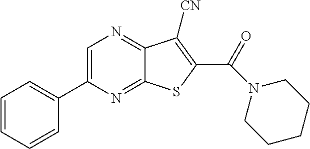 [SMILES: N#Cc1c(C(=O)N2CCCCC2)sc2nc(-c3ccccc3)cnc12]

MS (ESI) m/z (M+H)⁺=349.0. ¹H NMR (400 MHz, DMSO-d₆) δ 9.60 (s, 1H), 8.30-8.27 (m, 2H), 7.64-7.58 (m, 3H), 3.68-3.51 (m, 4H), 1.66-1.60 (m, 6H).

### Example 34: Preparation of (7-cyclopropyl-3-(2-methylpyridin-4-yl)thieno[2,3-b]pyrazin-6-yl)(piperidin-1-yl)methanone

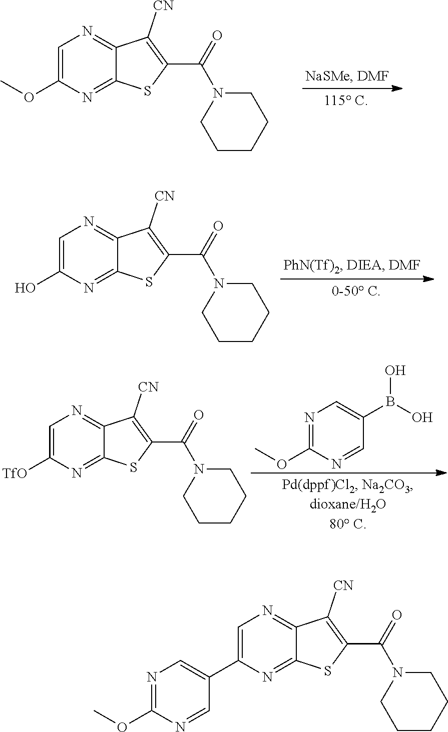

### Step 1: Preparation of 3-hydroxy-6-(piperidine-1-carbonyl)thieno[2,3-b]pyrazine-7-carbonitrile

3-methoxy-6-(piperidine-1-carbonyl)thieno[2,3-b]pyrazine-7-carbonitrile (1.2 g) was weighed and dissolved in N,N-dimethylformamide (5 mL), into which sodium methyl mercaptide (20% aqueous solution, 3 mL) was added to react at 115° C. for 1.5 h. The reaction was quenched by adding water, adjusted to an acidic pH value with 2 M hydrochloric acid, extracted with ethyl acetate for twice, washed with saturated brine for twice, dried over anhydrous sodium sulfate, and purified by a silica gel column chromatography to obtain a crude product of the title compound (1.2 g). MS (ESI) m/z (M+H)⁺=289.0.

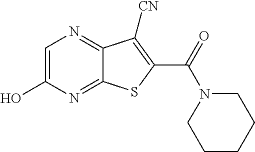 [SMILES: N#Cc1c(C(=O)N2CCCCC2)sc2nc(O)cnc12]

### Step 2: Preparation of 7-cyano-6-(piperidine-1-carbonyl)thieno[2,3-b]pyrazin-3-yltrifluoromethanesulfonate

(7-cyano-3-hydroxythieno[2,3-b]pyrazin-6-yl)(piperidin-1-yl)methanone (1.2 g) was weighed and dissolved in dichloromethane (12 mL), into which N,N-diisopropylethylamine (1.3 mL) and N-phenylbis(trifluoromethanesulphonimide) (2.9 g) were added to react overnight at 50° C. When TLC and LCMS monitored that the raw materials were completely reacted, the reaction was quenched by adding water, extracted with dichloromethane for twice, dried over anhydrous sodium sulfate, and purified by a silica gel column chromatography to obtain the title compound (0.9 g). MS (ESI) m/z (M+H)⁺=421.1.

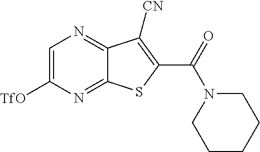 [SMILES: N#Cc1c(C(=O)N2CCCCC2)sc2nc(OS(=O)(=O)C(F)(F)F)cnc12]

### Step 3: Preparation of 3-(2-methoxypyrimidin-5-yl)-6-(piperidine-1-carbonyl)thieno[2,3-b]pyrazine-7-carbonitrile

7-cyano-6-(piperidine-1-carbonyl)thieno[2,3-b]pyrazin-3-yltrifluoromethanesulfonate (30 mg), 2-methoxy-5-pyrimidineboronic acid (22 mg), sodium carbonate (12 mg) and [1,1′-bis(diphenylphosphino)ferrocene]dichloropalladium (2 mg) were weighed, into which 1,4-dioxane (1 mL) and water (0.3 mL) were added, and after argon replacement for three times, a reaction was carried out at 80° C. for 1.5 h. When LCMS monitored that the raw materials were completely reacted, the reaction was quenched by adding water, extracted with ethyl acetate for twice, dried over anhydrous sodium sulfate, and separated by reverse phase preparative column to obtain the title compound (5.34 mg).

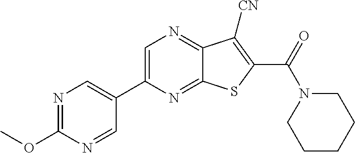 [SMILES: COc1ncc(-c2cnc3c(C#N)c(C(=O)N4CCCCC4)sc3n2)cn1]

MS (ESI) m/z (M+H)⁺=381.1. ¹H NMR (400 MHz, DMSO-d₆) δ 9.63 (s, 1H), 9.44 (s, 2H), 4.04 (s, 3H), 3.68-3.51 (m, 4H), 1.68-1.61 (in, 6H).

A series of compounds were prepared from corresponding commercial reagents and the products in the foregoing Preparation Examples and Examples as raw materials, by using a preparation method similar to that of the foregoing example, and structures and characterization data of the compounds are shown in Table 2.

TABLE 2

| | | Refer- | |
|---|---|---|---|
| | | ence | |
| | | example | |
| | | of | |
| Ex- | | prepara- | |
| am- | | tion | |
| ple | Structure | method | MS (M + H)⁺ & ¹H NMR |
| 35 | 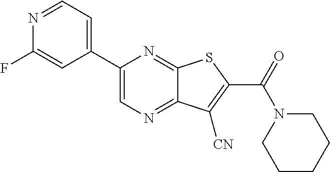 [SMILES: N#Cc1c(C(=O)N2CCCCC2)sc2nc(-c3ccnc(F)c3)cnc12] | Example 34 | MS (ESI) m/z (M + H)⁺ = 368.1, ¹H NMR (400 MHz, DMSO-d₆) δ 9.75 (s, 1H), 8.49 (d, J = 5.2 Hz, 1H), 8.23 (d, J = 3.8 Hz, 1H), 8.06 (s, 1H), 3.70-3.50 (m, 4H), 1.69-1.59 (m, 6H) |
| 36 | 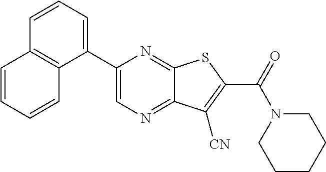 [SMILES: N#Cc1c(C(=O)N2CCCCC2)sc2nc(-c3cccc4ccccc34)cnc12] | Example 34 | MS (ESI) m/z (M + H)⁺ = 399.1, ¹H NMR (400 MHz, DMSO-d₆) δ 9.27 (d, J = 2.1 Hz, 1H), 8.17-8.15 (d, J = 8.1 Hz, 2H), 8.10-8.09 (d, J = 7.9 Hz, 1H), 7.85-7.74 (d, J = 7.0 Hz, 1H), 7.74-7.70 (t, J = 7.7 Hz, 1H), 7.65-7.58 (p, J = 7.1 Hz, 2H), 3.71-3.55 (d, J = 64.7 Hz, 4H), 1.69-1.63 (m, 6H) |
| 37 | 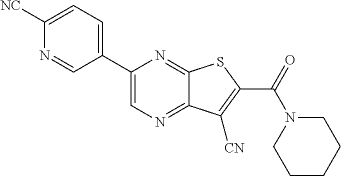 [SMILES: N#Cc1ccc(-c2cnc3c(C#N)c(C(=O)N4CCCCC4)sc3n2)cn1] | Example 34 | MS (ESI) m/z (M + H)⁺ = 375.1, ¹H NMR (400 MHz, DMSO-d₆) δ 9.76 (s, 1H), 9.62 (d, J = 2.1 Hz, 1H), 8.90 (d, J = 8.1 Hz, 1H), 8.30 (d, J = 8.2 Hz, 1H), 3.60-3.50 (m, 4H), 1.67-1.61 (m, 6H) |
| 38 | 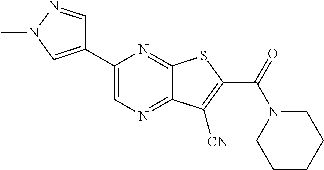 [SMILES: Cn1cc(-c2cnc3c(C#N)c(C(=O)N4CCCCC4)sc3n2)cn1] | Example 34 | MS (ESI) m/z (M + H)⁺ = 53.1, ¹H NMR (400 MHz, DMSO-d₆) δ 9.29 (s, 1H), 8.63 (s, 1H), 8.27 (s, 1H), 3.95 (s, 3H), 3.66-3.52 (m, 4H), 1.66-1.60 (m, 6H) |
| 39 | 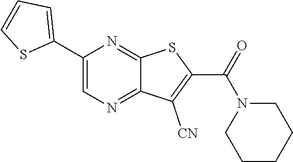 [SMILES: N#Cc1c(C(=O)N2CCCCC2)sc2nc(-c3cccs3)cnc12] | Example 34 | MS (ESI) m/z (M + H)⁺ = 355.1, ¹H NMR (400 MHz, DMSO-d₆) δ 9.56 (s, 1H), 8.23-8.22 (d, J = 3.7 Hz, 1H), 7.91-7.90 (d, J = 5.0 Hz, 1H), 7.33-7.31 (t, J = 4.4 Hz, 1H), 3.67-3.52 (m, 4H), 1.67-1.61 (m, 6H) |
| 40 | 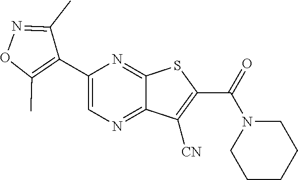 [SMILES: Cc1noc(C)c1-c1cnc2c(C#N)c(C(=O)N3CCCCC3)sc2n1] | Example 34 | MS (ESI) m/z (M + H)⁺ = 368.1, ¹H NMR (400 MHz, DMSO-d₆) δ 9.15 (s, 1H), 3.68-3.50 (m, 4H), 2.69 (s, 3H), 2.47 (s, 3H), 1.68- 1.61 (m, 6H) |
| 41 | 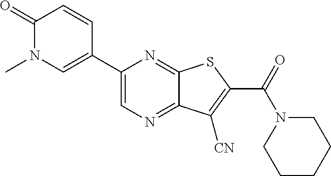 [SMILES: Cn1cc(-c2cnc3c(C#N)c(C(=O)N4CCCCC4)sc3n2)ccc1=O] | Example 34 | MS (ESI) m/z (M + H)⁺ = 380.1, ¹H NMR (400 MHz, DMSO-d₆) δ 9.42 (s, 1H), 8.90-8.89 (m, 1H), 8.32-8.29 (dt, J = 9.6, 1.6 Hz, 1H), 6.61-6.58 (d, J = 9.6 Hz, 1H), 3.66-3.51 (m, 7H), 1.69-1.60 (m, 6H) |
| 42 | 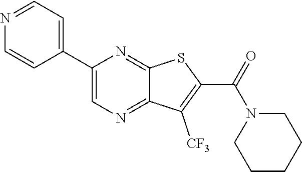 [SMILES: O=C(c1sc2nc(-c3ccncc3)cnc2c1C(F)(F)F)N1CCCCC1] | Example 34 | MS (ESI) m/z (M + H)⁺ = 350.1, ¹H NMR (400 MHz, DMSO-d₆) δ 9.72 (d, J = 1.6 Hz, 1H), 8.84-8.82 (m, 2H), 8.26-8.24 (q, J = 2.0 Hz, 2H), 3.69-3.50 (m, 4H), 1.67-1.61 (m, 6H) |
| 43 | 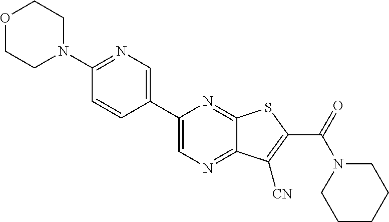 [SMILES: N#Cc1c(C(=O)N2CCCCC2)sc2nc(-c3ccc(N4CCOCC4)nc3)cnc12] | Example 34 | MS (ESI) m/z (M + H)⁺ = 435.2, ¹H NMR (400 MHz, DMSO-d₆) δ 9.52 (s, 1H), 9.07-9.06 (d, J = 2.5 Hz, 1H), 8.42-8.39 (dd, J = 9.1, 2.5 Hz, 1H), 7.04-7.02 (d, J = 9.1 Hz, 1H), 3.74-3.62 (m, 12H), 1.68-1.61 (m, 6H) |
| 44 | 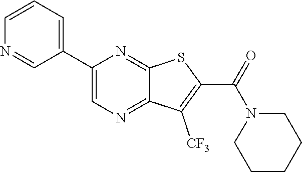 [SMILES: O=C(c1sc2nc(-c3cccnc3)cnc2c1C(F)(F)F)N1CCCCC1] | Example 34 | MS (ESI) m/z (M + H)⁺ = 350.1, ¹H NMR (400 MHz, DMSO-d₆) δ 9.68-9.67 (d, J = 1.4 Hz, 1H), 9.46 (s, 1H), 8.78-8.77 (d, J = 4.7 Hz, 1H), 8.65-8.63 (dd, J = 8.2, 2.0 Hz, 1H), 7.67-7.62 (dd, J = 8.0, 4.8 Hz, 1H), 3.74-3.48 (m, 4H), 1.71- 1.55 (m, 6H) |
| 45 | 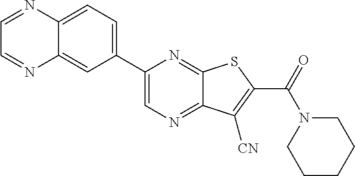 [SMILES: N#Cc1c(C(=O)N2CCCCC2)sc2nc(-c3ccc4nccnc4c3)cnc12] | Example 34 | MS (ESI) m/z (M + H)⁺ = 401.1, ¹H NMR (400 MHz, DMSO-d₆) δ 9.89 (s, 1H), 9.08 (d, J = 1.8 Hz, 1H), 9.06 (dd, J = 4.3, 1.8 Hz, 2H), 8.76 (dd, J = 8.7, 1.9 Hz, 1H), 8.32 (d, J = 8.9 Hz, 1H), 3.70 (m, 2H), 3.53 (m, 2H), 1.71-1.59 (m, 6H) |
| 46 | 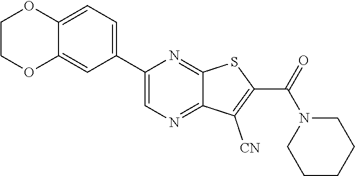 [SMILES: N#Cc1c(C(=O)N2CCCCC2)sc2nc(-c3ccc4c(c3)OCCO4)cnc12] | Example 34 | MS (ESI) m/z (M + H)⁺ = 407.1, ¹H NMR (400 MHz, DMSO-d₆) δ 9.53 (s, 1H), 7.81 (d, J = 8.3 Hz, 2H), 7.07 (d, J = 8.3 Hz, 1H), 4.38- 4.31 (m, 4H), 3.67 (m, 2H), 3.51 (m, 2H), 1.64 (d, J = 23.9 Hz, 6H) |
| 47 | 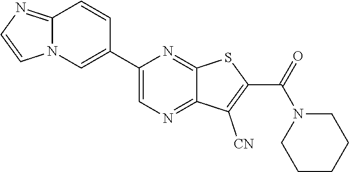 [SMILES: N#Cc1c(C(=O)N2CCCCC2)sc2nc(-c3ccc4nccn4c3)cnc12] | Example 34 | MS (ESI) m/z (M + H)⁺ = 389.1, ¹H NMR (400 MHz, DMSO-d₆) δ 9.64 (s, 1H), 9.60 (s, 1H), 8.10 (d, J = 7.4 Hz, 2H), 7.77 (d, J = 9.5 Hz, 1H), 7.70 (s, 1H), 3.69 (m, 2H), 3.52 (m, 2H), 1.71-1.56 (m, 6H) |
| 48 | 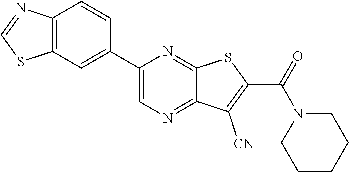 [SMILES: N#Cc1c(C(=O)N2CCCCC2)sc2nc(-c3ccc4ncsc4c3)cnc12] | Example 34 | MS (ESI) m/z (M + H)⁺ = 406.1, ¹H NMR (400 MHz, DMSO-d₆) δ 9.70 (s, 1H), 9.56 (s, 1H), 9.16 (s, 1H), 8.47 (d, J = 8.6 Hz, 1H), 8.29 (d, J = 8.8 Hz, 1H), 3.69 (m, 2H), 3.53 (m, 2H), 1.71-1.59 (m, 6H) |
| 49 | 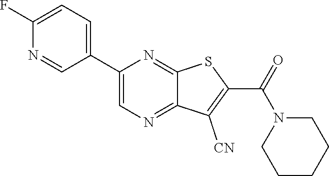 [SMILES: N#Cc1c(C(=O)N2CCCCC2)sc2nc(-c3ccc(F)nc3)cnc12] | Example 34 | MS (ESI) m/z (M + H)⁺ = 368.1, ¹H NMR (400 MHz, DMSO-d₆) δ 9.67 (s, 1H), 9.16 (s, 1H), 8.84 (t, J = 8.6 Hz, 1H), 7.47 (d, J = 8.9 Hz, 1H), 3.69 (m, 2H), 3.51 (m, 2H), 1.64 (m, 6H) |
| 50 | 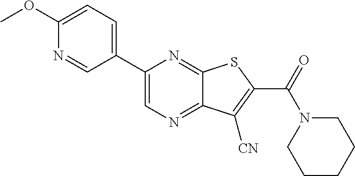 [SMILES: COc1ccc(-c2cnc3c(C#N)c(C(=O)N4CCCCC4)sc3n2)cn1] | Example 34 | MS (ESI) m/z (M + H)⁺ = 380.1, ¹H NMR (400 MHz, DMSO-d₆) δ 9.60 (s, 1H), 9.11 (d, J = 2.4 Hz, 1H), 8.56 (dd, J = 8.8, 2.4 Hz, 1H), 7.05 (d, J = 8.8 Hz, 1H), 3.97 (s, 3H), 3.68 (m, 2H), 3.51 (m, 2H), 1.72 - 1.55 (m, 6H) |
| 51 | 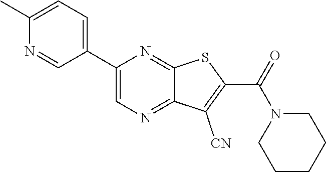 [SMILES: Cc1ccc(-c2cnc3c(C#N)c(C(=O)N4CCCCC4)sc3n2)cn1] | Example 34 | MS (ESI) m/z (M + H)⁺ = 364.1, ¹H NMR (400 MHz, DMSO-d₆) δ 9.64 (s, 1H), 9.34 (s, 1H), 8.54 (d, J = 8.8 Hz, 1H), 7.51 (d, J = 8.2 Hz, 1H), 3.69 (m, 2H), 3.51 (m, 2H), 2.59 (s, 3H), 1.64 (m, 6H) |
| 52 | 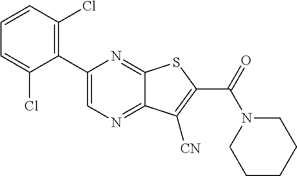 [SMILES: N#Cc1c(C(=O)N2CCCCC2)sc2nc(-c3c(Cl)cccc3Cl)cnc12] | Example 34 | MS (ESI) m/z (M + H)⁺ = 417.1, ¹H NMR (400 MHz, DMSO-d₆) δ 9.15 (s, 1H), 7.73 (d, J = 7.8 Hz, 2H), 7.64 (dd, J = 8.9, 7.2 Hz, 1H), 3.69 (m, 2H), 3.55 (m, 2H), 1.71-1.57 (m, 6H) |
| 53 | 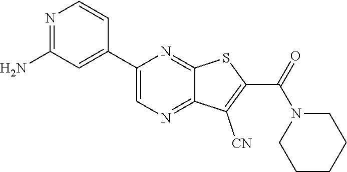 [SMILES: N#Cc1c(C(=O)N2CCCCC2)sc2nc(-c3ccnc(N)c3)cnc12] | Example 34 | MS (ESI) m/z (M + H)⁺ = 365.1, ¹H NMR (400 MHz, DMSO-d₆) δ 9.50 (s, 1H), 8.13 (d, J = 5.3 Hz, 1H), 7.32-7.28 (m, 2H), 6.25 (s, 2H), 3.69 (m, 2H), 3.51 (m, 2H), 1.72-1.55 (m, 6H) |
| 54 | 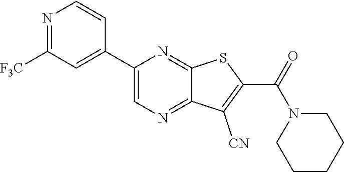 [SMILES: N#Cc1c(C(=O)N2CCCCC2)sc2nc(-c3ccnc(C(F)(F)F)c3)cnc12] | Example 34 | MS (ESI) m/z (M + H)⁺ = 418.1, ¹H NMR (400 MHz, DMSO-d₆) δ 9.85 (d, J = 1.5 Hz, 1H), 9.03 (d, J = 5.1 Hz, 1H), 8.71 (s, 1H), 8.60 (d, J = 5.1 Hz, 1H), 3.70 (m, 2H), 3.51 (m, 2H), 1.64 (m, 6H) |
| 55 | 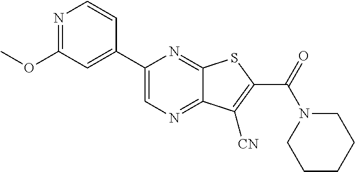 [SMILES: COc1cc(-c2cnc3c(C#N)c(C(=O)N4CCCCC4)sc3n2)ccn1] | Example 34 | MS (ESI) m/z (M + H)⁺ = 380.1, ¹H NMR (400 MHz, DMSO-d₆) δ 9.69 (d, J = 2.0 Hz, 1H), 8.41 (d, J = 5.2 Hz, 1H), 7.84 (d, J = 5.4 Hz, 1H), 7.69 (s, 1H), 3.96 (s, 3H), 3.75-3.64 (m, 2H), 3.50 (m, 2H), 1.63 (m, 6H) |
| 56 | 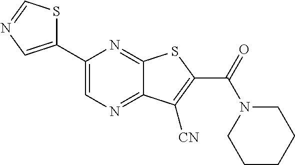 [SMILES: N#Cc1c(C(=O)N2CCCCC2)sc2nc(-c3cncs3)cnc12] | Example 34 | MS (ESI) m/z (M + H)⁺ = 356.1, ¹H NMR (400 MHz, DMSO-d₆) δ 9.65 (s, 1H), 9.36 (s, 1H), 8.99 (d, J = 0.6 Hz, 1H), 3.68 (m, 2H), 3.50 (m, 2H), 1.70-1.56 (m, 6H) |
| 57 | 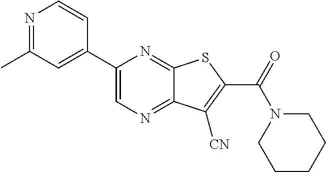 [SMILES: Cc1cc(-c2cnc3c(C#N)c(C(=O)N4CCCCC4)sc3n2)ccn1] | Example 34 | MS (ESI) m/z (M + H)⁺ = 364.1, ¹H NMR (400 MHz, DMSO-d₆) δ 9.68 (s, 1H), 8.68 (d, J = 5.1 Hz, 1H), 8.16-8.11 (m, 1H), 8.04 (dd, J = 5.2, 1.7 Hz, 1H), 3.69 (m, 2H), 3.50 (m, 2H), 2.62 (s, 3H), 1.72-1.54 (m, 6H) |
| 58 | 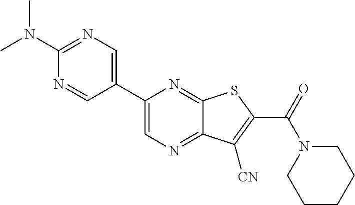 [SMILES: CN(C)c1ncc(-c2cnc3c(C#N)c(C(=O)N4CCCCC4)sc3n2)cn1] | Example 34 | MS (ESI) m/z (M + H)⁺ = 394.1, ¹H NMR (400 MHz, DMSO-d₆) δ 9.51 (s, 1H), 9.22 (s, 2H), 3.67 (m, 2H), 3.51 (m, 2H), 3.24 (s, 6H), 1.63 (m, 6H) |
| 59 | 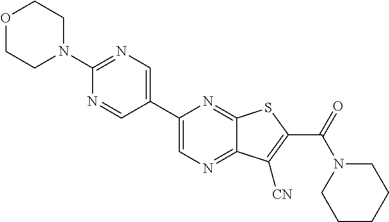 [SMILES: N#Cc1c(C(=O)N2CCCCC2)sc2nc(-c3cnc(N4CCOCC4)nc3)cnc12] | Example 34 | MS (ESI) m/z (M + H)⁺ = 436.1, ¹H NMR (400 MHz, DMSO-d₆) δ 9.53 (s, 1H), 9.25 (s, 2H), 3.86 (t, J = 4.9 Hz, 4H), 3.70 (dd, J = 7.0, 2.6 Hz, 4H), 3.64 (m, 2H), 3.51 (m, 2H), 1.69- 1.58 (m, 6H) |
| 60 | 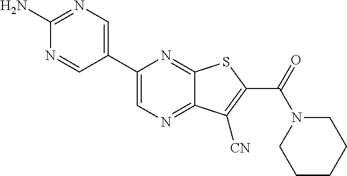 [SMILES: N#Cc1c(C(=O)N2CCCCC2)sc2nc(-c3cnc(N)nc3)cnc12] | Example 34 | MS (ESI) m/z (M + H)⁺ = 366.1, ¹H NMR (400 MHz, DMSO-d₆) δ 9.50 (s, 1H), 9.13 (s, 2H), 7.39 (s, 2H), 3.67 (m, 2H), 3.51 (m, 2H), 1.63 (m, 6H) |
| 61 | 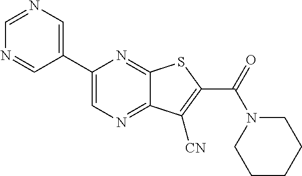 [SMILES: N#Cc1c(C(=O)N2CCCCC2)sc2nc(-c3cncnc3)cnc12] | Example 34 | MS (ESI) m/z (M + H)⁺ = 351.1, ¹H NMR (400 MHz, DMSO-d₆) δ 9.73 (s, 1H), 9.64 (s, 2H), 9.38 (s, 1H), 3.69 m, 2H), 3.51 (m, 2H), 1.64 (m, 6H) |
| 62 | 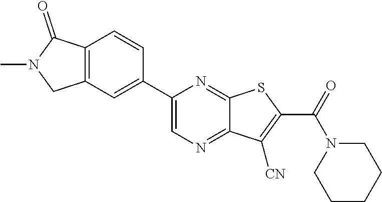 [SMILES: CN1Cc2cc(-c3cnc4c(C#N)c(C(=O)N5CCCCC5)sc4n3)ccc2C1=O] | Example 34 | MS (ESI) m/z (M + H)⁺ = 418.1, ¹H NMR (400 MHz, DMSO-d₆) δ 9.67 (s, 1H), 8.51 (s, 1H), 8.40 (d, J = 8.1 Hz, 1H), 7.87 (d, J = 8.0 Hz, 1H), 4.61 (s, 2H), 3.69 (m, 2H), 3.52 (m, 2H), 3.13 (s, 3H), 1.64 (m, 6H) |

### Example 63: Preparation of (3-phenyl-7-(trifluoromethyl)thieno[2,3-b]pyrazin-6-yl)(piperidin-1-yl)methanone

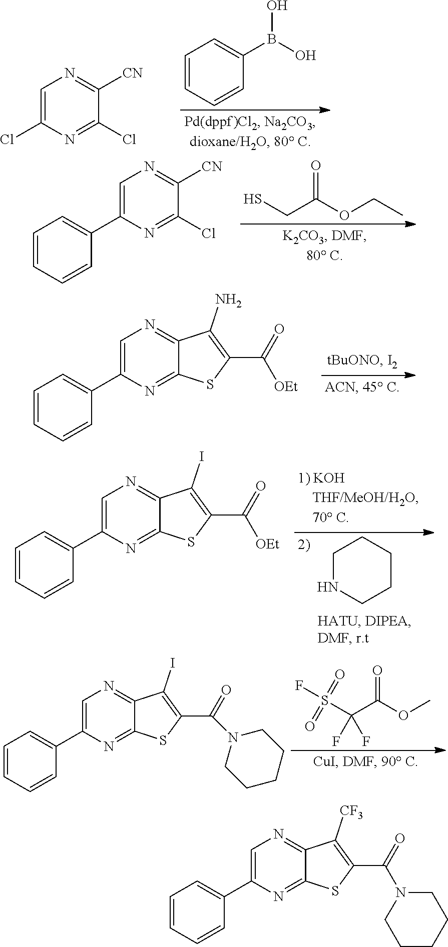

### Step 1: Preparation of 3-chloro-5-phenylpyrazine-2-carbonitrile

3,5-dichloropyrazine-2-carbonitrile (2.5 g), phenylboronic acid (1.95 g), sodium carbonate (1.84 g) and [1,1′-bis(diphenylphosphino)ferrocene]dichloropalladium (0.53 g) were weighed and dissolved in a mixed solvent of 1,4-dioxane (20 mL) and water (5 mL), and after nitrogen replacement for three times, a reaction was carried out at 80° C. for 2 h. TLC monitored that the raw materials were completely reacted. It was cooled to room temperature, and filtered. Filtrate was added with water and extracted with ethyl acetate. Organic phases were combined, dried over anhydrous sodium sulfate, and evaporated under reduced pressure to remove the solvent. A residue was purified by a silica gel column to obtain a crude product of the title compound (3.1 g). MS (ESI) m/z (M+H)⁺=216.0.

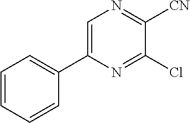 [SMILES: N#Cc1ncc(-c2ccccc2)nc1Cl]

### Step 2: Preparation of ethyl 7-amino-3-phenylthieno[2,3-b]pyrazine-6-carboxylate

3-chloro-5-phenylpyrazine-2-carbonitrile (80 mg) was weighed and dissolved in N,N-dimethylformamide (2 mL), into which potassium carbonate (120 mg) and ethyl mercaptoacetate (54 μL) were added to react overnight at 80° C. LC-MS monitored that the reaction was completed. It was cooled to room temperature, quenched by adding water, and extracted with ethyl acetate for three times. Organic phases were combined, dried over anhydrous sodium sulfate, concentrated under reduced pressure, and purified by a silica gel column to obtain the title compound (90 mg). MS (ESI) m/z (M+H)⁺=300.1.

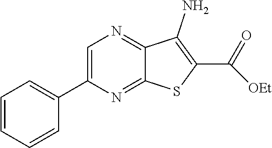 [SMILES: CCOC(=O)c1sc2nc(-c3ccccc3)cnc2c1N]

### Step 3: Preparation of ethyl 7-iodo-3-phenylthieno[2,3-b]pyrazine-6-carboxylate (BT-120-348-062)

Iodine (0.76 g) and tert-butyl nitrite (0.18 mL) were weighed and dissolved in dry acetonitrile (10 mL), into which ethyl 7-amino-3-phenylthieno[2,3-b]pyrazine-6-carboxylate (300 mg) was added under the protection of nitrogen, copper bromide (490 mg) was added thereto, and then tert-butyl nitrite (0.3 mL) was added dropwise to react at 45° C. for 2 h. TLC monitored that the reaction was completed. It was cooled to room temperature, quenched by adding a saturated sodium bisulfite solution, then adjusted to pH 8 with a saturated sodium bicarbonate solution, and extracted with ethyl acetate. Organic phases were combined and dried over anhydrous sodium sulfate. Evaporation was performed under reduced pressure to remove the solvent, and purification was performed by a silica gel column to obtain the title compound (80 mg). MS (ESI) m/z (M+H)⁺=411.1.

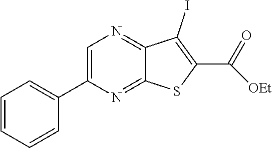 [SMILES: CCOC(=O)c1sc2nc(-c3ccccc3)cnc2c1I]

### Step 4: Preparation of (7-iodo-3-phenylthieno[2,3-b]pyrazin-6-yl)(piperidin-1-yl)methanone (BT-120-348-063)

Ethyl 7-iodo-3-phenylthieno[2,3-b]pyrazine-6-carboxylate (80 mg) was weighed and dissolved in tetrahydrofuran (1 mL), methanol (0.3 mL) and water (0.3 mL), into which potassium hydroxide (34 mg) was added to react at 70° C. for 2 h. LCMS monitored that the reaction was completed. It was cooled to room temperature, added with water, and extraction was performed with ethyl acetate for three times to discard an organic phase. An aqueous phase was adjusted to pH 2 with 2 M diluted hydrochloric acid, and then extracted with ethyl acetate. Organic phases were combined, dried over anhydrous sodium sulfate, and evaporated under reduced pressure to remove the solvent to obtain a crude product. The crude product was dissolved in N,N-dimethylformamide (2 mL), into which diisopropylethylamine (65 μL), piperidine (25 μL) and 2-(7-azabenzotriazol-1-yl)-N,N,N′,N′-tetramethyluronium hexafluorophosphate (114 mg) were sequentially added under an ice water bath, then transferred to room temperature, and stirred for 2 h. LCMS monitored that a reaction was completed. The reaction was quenched by adding water, and extracted with ethyl acetate for three times. Organic phases were combined, dried over anhydrous sodium sulfate, and concentrated under reduced pressure. A residue was purified by a silica gel column to obtain the title compound (30 mg). MS (ESI) m/z (M+H)⁺=449.9.

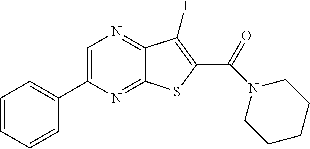 [SMILES: O=C(c1sc2nc(-c3ccccc3)cnc2c1I)N1CCCCC1]

### Step 5: Preparation of 3-phenyl-6-(piperidine-1-carbonyl)thieno[2,3-b]pyrazine-7-carbonitrile (BT-120-348-065)

(7-iodo-3-phenylthieno[2,3-b]pyrazin-6-yl)(piperidin-1-yl)methanone (15 mg), methyl 2,2-difluoro-2-(fluorosulfonyl)acetate (20 mg) and cuprous iodide (1 mg) were weighed and dissolved in N,N-dimethylformamide (1 mL) to react at 90° C. for 3 h under the protection of nitrogen. LCMS monitored that the reaction was completed. It was cooled to room temperature, the reaction was quenched by adding water, and extracted with ethyl acetate for three times. Organic phases were combined, dried over anhydrous sodium sulfate, concentrated under reduced pressure, and purified by Prep-HPLC to obtain the title compound (2.27 mg).

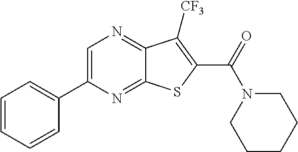 [SMILES: O=C(c1sc2nc(-c3ccccc3)cnc2c1C(F)(F)F)N1CCCCC1]

MS (ESI) m/z (M+H)+: 392.1. ¹H NMR (400 MHz, DMSO-d₆) δ 9.58-9.56 (t, J=2.3 Hz, 1H), 8.28-8.26 (d, J=7.0 Hz, 2H), 7.64-7.60 (m, 3H), 3.67-3.66 (m, 2H), 3.35-3.33 (m, 2H), 1.65-1.59 (m, 4H), 1.48 (m, 2H).

### Example 64: Preparation of 3-(2-methoxypyrimidin-5-yl)-6-(piperidine-1-carbonyl)thieno[2,3-b]pyrazine-7-carbonitrile

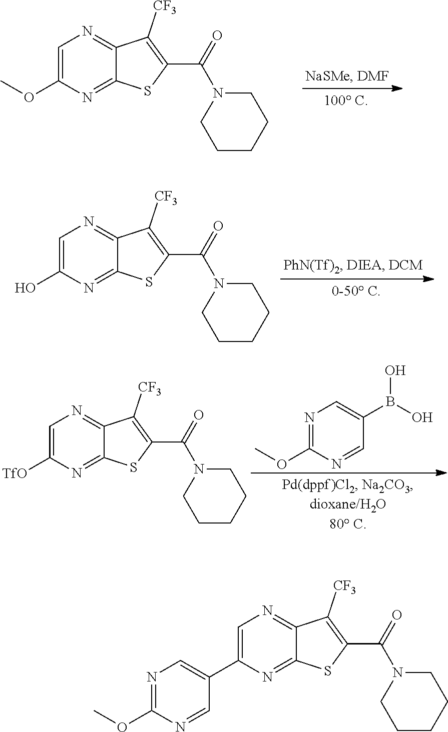

### Step 1: Preparation of (3-hydroxy-7-(trifluoromethyl)thieno[2,3-b]pyrazin-6-yl)(piperidin-1-yl)methanone

(3-methoxy-7-(trifluoromethyl)thieno[2,3-b]pyrazin-6-yl)(piperidin-1-yl)methanone (845 mg) obtained in Preparation Example 3 was weighed and dissolved in N,N-dimethylformamide (10 mL), into which sodium methyl mercaptide (20% aqueous solution, 1.46 mL) was added to react overnight at 100° C. The reaction was quenched by adding water, adjusted to an acidic pH value with 2 M hydrochloric acid, extracted with ethyl acetate for twice, washed with saturated brine for twice, dried over anhydrous sodium sulfate, and purified by a silica gel column chromatography to obtain the title compound (800 mg). MS (ESI) m/z (M+H)⁺=332.0.

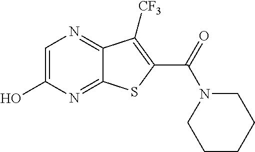 [SMILES: O=C(c1sc2nc(O)cnc2c1C(F)(F)F)N1CCCCC1]

### Step 2: Preparation of 6-(piperidine-1-carbonyl)-7-(trifluoromethyl)thieno[2,3-b]pyrazin-3-yltrifluoromethanesulfonate

(3-hydroxy-7-(trifluoromethyl)thieno[2,3-b]pyrazin-6-yl)(piperidin-1-yl)methanone (800 mg) was weighed and dissolved in dichloromethane (12 mL), into which N,N-diisopropylethylamine (0.84 mL) and N-phenylbis(trifluoromethanesulphonimide) (1.72 g) were added to react overnight at 50° C. When TLC and LCMS monitored that the raw materials were completely reacted, the reaction was quenched by adding water, extracted with dichloromethane for twice, dried over anhydrous sodium sulfate, and purified by a silica gel column chromatography to obtain the title compound (560 mg). MS (ESI) m/z (M+H)⁺=464.0.

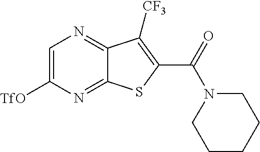 [SMILES: O=C(c1sc2nc(OS(=O)(=O)C(F)(F)F)cnc2c1C(F)(F)F)N1CCCCC1]

### Step 3: Preparation of (3-(2-methoxypyrimidin-5-yl)-7-(trifluoromethyl)thieno[2,3-b]pyrazin-6-yl)(piperidin-1-yl)methanone

6-(piperidine-1-carbonyl)-7-(trifluoromethyl)thieno[2,3-b]pyrazin-3-yltrifluoromethanesulfonate (25 mg), 2-methoxy-5-pyrimidineboronic acid (9 mg), sodium carbonate (8 mg) and [1,1′-bis(diphenylphosphino)ferrocene]dichloropalladium (2 mg) were weighed, into which 1,4-dioxane (1.5 mL) and water (0.3 mL) were added, and after argon replacement for three times, a reaction was carried out at 80° C. for 1.5 h. When LCMS monitored that the raw materials were completely reacted, the reaction was quenched by adding water, extracted with ethyl acetate for twice, dried over anhydrous sodium sulfate, and separated by a reverse phase preparative column to obtain the title compound (8.30 mg).

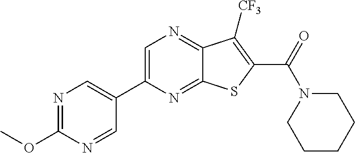 [SMILES: COc1ncc(-c2cnc3c(C(F)(F)F)c(C(=O)N4CCCCC4)sc3n2)cn1]

MS (ESI) m/z (M+H)⁺=424.1. ¹H NMR (400 MHz, DMSO-d₆) δ 9.59 (s, 1H), 9.43 (s, 2H), 4.04 (s, 3H), 3.66 (t, J=5.3 Hz, 2H), 3.32 (m, 2H), 1.64 (d, J=6.3 Hz, 2H), 1.59 (t, J=5.7 Hz, 2H), 1.47 (dd, J=10.5, 5.6 Hz, 2H).

A series of compounds were prepared from corresponding commercial reagents and the products in the foregoing Preparation Examples and Examples as raw materials, by using preparation methods similar to those of the foregoing examples, and structures and characterization data of the compounds are shown in Table 3.

TABLE 3

| | | Reference | |
|---|---|---|---|
| | | examples | |
| Ex- | | of | |
| am- | | preparation | |
| ple | Structure | methods | MS (M + H)⁺ & ¹H NMR |
| 65 | 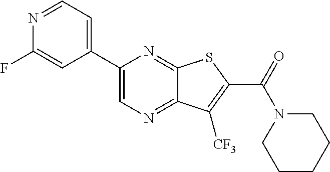 [SMILES: O=C(c1sc2nc(-c3ccnc(F)c3)cnc2c1C(F)(F)F)N1CCCCC1] | Example 64 | MS (ESI) m/z (M + H)⁺ = 411.1, ¹H NMR (400 MHz, DMSO-d₆) δ 9.72 (s, 1H), 8.50 (d, J = 5.2 Hz, 1H), 8.22 (dt, J = 5.3, 1.7 Hz, 1H), 8.05 (s, 1H), 3.67 (t, J = 5.4 Hz, 2H), 3.36 (m, 2H), 1.65 (t, J = 5.7 Hz, 2H), 1.62-1.56 (m, 2H), 1.48 (t, J = 5.6 Hz, 2H) |
| 66 | 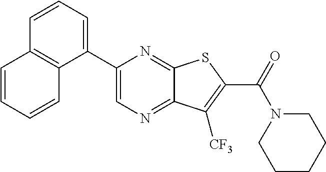 [SMILES: O=C(c1sc2nc(-c3cccc4ccccc34)cnc2c1C(F)(F)F)N1CCCCC1] | Example 64 | MS (ESI) m/z (M + H)⁺ = 442.1, ¹H NMR (400 MHz, DMSO-d₆) δ 9.16 (s, 1H), 8.20-8.16 (m, 1H), 8.10 (d, J = 8.3 Hz, 1H), 8.04 (dd, J = 6.9, 2.6 Hz, 1H), 7.82 (dd, J = 7.1, 1.2 Hz, 1H), 7.70 (d, J = 7.8 Hz, 1H), 7.63 - 7.56 (m, 2H), 3.81 (t, J = 5.4 Hz, 2H), 3.47 (t, J = 5.6 Hz, 2H), 1.77 (t, J = 10.6 Hz, 4H), 1.66 (d, J = 4.9 Hz, 2H) |
| 67 | 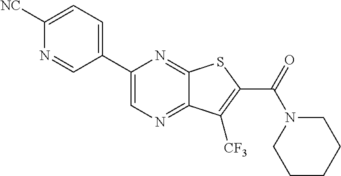 [SMILES: N#Cc1ccc(-c2cnc3c(C(F)(F)F)c(C(=O)N4CCCCC4)sc3n2)cn1] | Example 64 | MS (ESI) m/z (M + H)⁺ = 418.1, ¹H NMR (400 MHz, DMSO-d₆) δ 9.76 (s, 1H), 9.62 (d, J = 2.1 Hz, 1H), 8.90 (d, J = 8.1 Hz, 1H), 8.30 (d, J = 8.2 Hz, 1H), 3.60-3.50 (m, 4H), 1.67- 1.61 (m, 6H) |
| 68 | 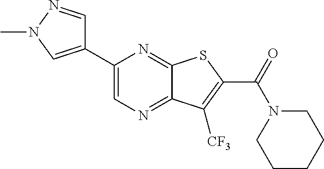 [SMILES: Cn1cc(-c2cnc3c(C(F)(F)F)c(C(=O)N4CCCCC4)sc3n2)cn1] | Example 64 | MS (ESI) m/z (M + H)⁺ = 396.1, ¹H NMR (400 MHz, DMSO-d₆) δ 9.26 (s, 1H), 8.61 (s, 1H), 8.25 (s, 1H), 3.95 (s, 3H), 3.65 (t, J = 5.3 Hz, 2H), 3.32 (m, 2H), 1.67-1.55 (m, 4H), 1.50-1.44 (m, 2H) |
| 69 | 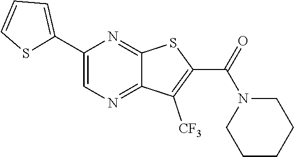 [SMILES: O=C(c1sc2nc(-c3cccs3)cnc2c1C(F)(F)F)N1CCCCC1] | Example 64 | MS (ESI) m/z (M + H)⁺ = 398.1, ¹H NMR (400 MHz, DMSO-d₆) δ 9.53 (s, 1H), 8.18 (dd, J = 3.8, 1.1 Hz, 1H), 7.89 (dd, J = 5.0, 1.1 Hz, 1H), 7.31 (dd, J = 5.1, 3.7 Hz, 1H), 3.65 (t, J = 5.3 Hz, 2H), 3.33 (d, J = 4.8 Hz, 2H), 1.64 (q, J = 5.7 Hz, 2H), 1.61-1.55 (m, 2H), 1.47 (p, J = 6.0 Hz, 2H) |
| 70 | 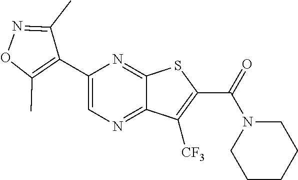 [SMILES: Cc1noc(C)c1-c1cnc2c(C(F)(F)F)c(C(=O)N3CCCCC3)sc2n1] | Example 64 | MS (ESI) m/z (M + H)⁺ = 411.1, ¹H NMR (400 MHz, DMSO-d₆) δ 9.13 (s, 1H), 3.66 (t, J = 5.3 Hz, 2H), 3.33 (d, J = 5.3 Hz, 2H), 2.68 (s, 3H), 2.47 (s, 3H), 1.64 (q, J = 5.8 Hz, 2H), 1.61-1.55 (m, 2H), 1.47 (t, J = 5.6 Hz, 2H) |
| 71 | 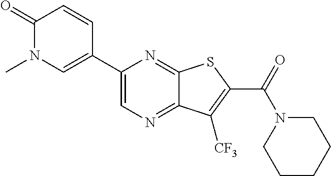 [SMILES: Cn1cc(-c2cnc3c(C(F)(F)F)c(C(=O)N4CCCCC4)sc3n2)ccc1=O] | Example 64 | MS (ESI) m/z (M + H)⁺ = 423.1, ¹H NMR (400 MHz, DMSO-d₆) δ 9.29 (s, 1H), 8.76 (d, J = 2.6 Hz, 1H), 8.43 (dd, J = 9.5, 2.7 Hz, 1H), 6.73 (d, J = 9.5 Hz, 1H), 3.79 (t, J = 5.4 Hz, 2H), 3.74 (s, 3H), 3.42 (t, J = 5.6 Hz, 2H), 1.75 (dq, J = 15.8, 5.9, 5.0 Hz, 4H), 1.62 (p, J = 5.7 Hz, 2H) |
| 72 | 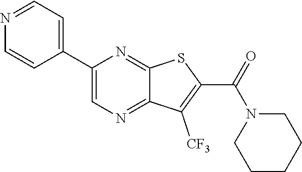 [SMILES: O=C(c1sc2nc(-c3ccncc3)cnc2c1C(F)(F)F)N1CCCCC1] | Example 64 | MS (ESI) m/z (M + H)⁺ = 393.1, ¹H NMR (400 MHz, DMSO-d₆) δ 9.68 (s, 1H), 8.82 (d, J = 5.3 Hz, 2H), 8.23 (d, J = 5.3 Hz, 2H), 3.67 (d, J = 5.6 Hz, 2H), 3.33 (m, 2H), 1.65 (t, J = 6.0 Hz, 2H), 1.62-1.55 (m, 2H), 1.48 (d, J = 7.1 Hz, 2H) |
| 73 | 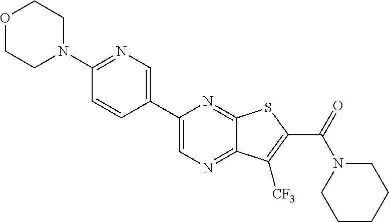 [SMILES: O=C(c1sc2nc(-c3ccc(N4CCOCC4)nc3)cnc2c1C(F)(F)F)N1CCCCC1] | Example 64 | MS (ESI) m/z (M + H)⁺ = 478.1, ¹H NMR (400 MHz, DMSO-d₆)) δ 9.50 (s, 1H), 9.05 (d, J = 2.4 Hz, 1H), 8.40 (dd, J = 9.1, 2.5 Hz, 1H), 7.04 (d, J = 9.1 Hz, 1H), 3.73 (t, J = 4.7 Hz, 4H), 3.63 (q, J = 5.4, 4.6 Hz, 6H), 3.32 (m, 2H), 1.64-1.58 (m, 4H), 1.47 (s, 2H) |
| 74 | 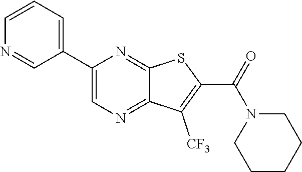 [SMILES: O=C(c1sc2nc(-c3cccnc3)cnc2c1C(F)(F)F)N1CCCCC1] | Example 64 | MS (ESI) m/z (M + H)⁺ = 393.1, ¹H NMR (400 MHz, DMSO-d₆) δ 9.49 (s, 1H), 9.42 (dd, J = 2.3, 0.9 Hz, 1H), 8.74-8.68 (m, 2H), 7.68 (ddd, J = 8.1, 4.9, 0.9 Hz, 1H), 3.80 (t, J = 5.4 Hz, 2H), 3.46 - 3.42 (m, 2H), 1.80-1.71 (m, 4H), 1.64 (q, J = 5.6, 5.2 Hz, 2H) |
| 75 | 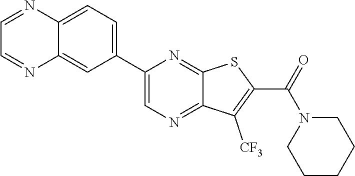 [SMILES: O=C(c1sc2nc(-c3ccc4nccnc4c3)cnc2c1C(F)(F)F)N1CCCCC1] | Example 64 | MS (ESI) m/z (M + H)⁺ = 441.1, ¹H NMR (400 MHz, DMSO-d₆) δ 9.67 (s, 1H), 9.01 (dd, J = 3.3, 1.9 Hz, 2H), 8.98 (d, J = 1.8 Hz, 1H), 8.78 (dd, J = 8.9, 2.1 Hz, 1H), 8.32 (d, J = 8.9 Hz, 1H), 3.81 (t, J = 5.4 Hz, 2H), 3.46 (t, J = 5.6 Hz, 2H), 1.80-1.73 (m, 4H), 1.67-1.62 (m, 2H) |
| 76 | 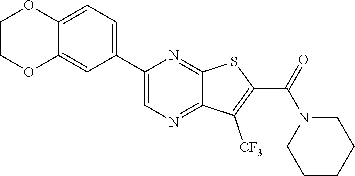 [SMILES: O=C(c1sc2nc(-c3ccc4c(c3)OCCO4)cnc2c1C(F)(F)F)N1CCCCC1] | Example 64 | MS (ESI) m/z (M + H)⁺ = 450.1, ¹H NMR (400 MHz, DMSO-d₆) δ 9.31 (s, 1H), 7.80-7.72 (m, 2H), 7.03 (d, J = 8.4 Hz, 1H), 4.38- 4.30 (m, 4H), 3.79 (t, J = 5.4 Hz, 2H), 3.42 (t, J = 5.6 Hz, 2H), 1.75 (dt, J = 16.4, 5.9 Hz, 4H), 1.62 (p, J = 5.6 Hz, 2H) |
| 77 | 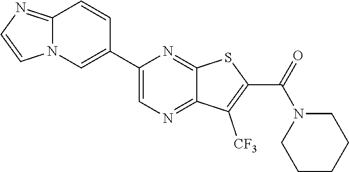 [SMILES: O=C(c1sc2nc(-c3ccc4nccn4c3)cnc2c1C(F)(F)F)N1CCCCC1] | Example 64 | MS (ESI) m/z (M + H)⁺ = 432.1, ¹H NMR (400 MHz, DMSO-d₆) δ 9.50-9.47 (m, 1H), 9.46 (s, 1H), 8.21 (dd, J = 9.5, 1.8 Hz, 1H), 8.03 (d, J = 1.4 Hz, 1H), 7.75 (d, J = 9.5 Hz, 1H), 7.69 (d, J = 1.4 Hz, 1H), 3.80 (t, J = 5.4 Hz, 2H), 3.44 (t, J = 5.6 Hz, 2H), 1.75 (dt, J = 11.4, 4.8 Hz, 4H), 1.64 (q, J = 5.6 Hz, 2H) |
| 78 | 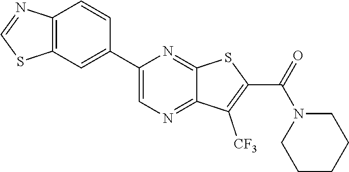 [SMILES: O=C(c1sc2nc(-c3ccc4ncsc4c3)cnc2c1C(F)(F)F)N1CCCCC1] | Example 64 | MS (ESI) m/z (M + H)⁺ = 449.1, ¹H NMR (400 MHz, DMSO-d₆) δ 9.70 (s, 1H), 9.56 (s, 1H), 9.16 (s, 1H), 8.47 (d, J = 8.6 Hz, 1H), 8.29 (d, J = 8.8 Hz, 1H), 3.69 (m, 2H), 3.53 (m, 2H), 1.71-1.59 (m, 6H) |
| 79 | 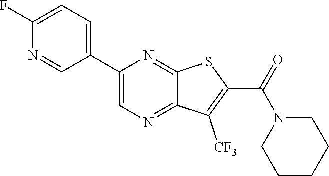 [SMILES: O=C(c1sc2nc(-c3ccc(F)nc3)cnc2c1C(F)(F)F)N1CCCCC1] | Example 64 | MS (ESI) m/z (M + H)⁺ = 411.1, ¹H NMR (400 MHz, DMSO-d₆) δ 9.46 (s, 1H), 9.10 (d, J = 2.5 Hz, 1H), 8.80 (ddd, J = 8.6, 7.5, 2.6 Hz, 1H), 7.31 (dd, J = 8.7, 2.6 Hz, 1H), 3.80 (t, J = 5.4 Hz, 2H), 3.46-3.41 (m, 2H), 1.81-1.70 (m, 4H), 1.63 (t, J = 5.7 Hz, 2H) |
| 80 | 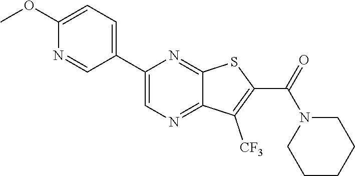 [SMILES: COc1ccc(-c2cnc3c(C(F)(F)F)c(C(=O)N4CCCCC4)sc3n2)cn1] | Example 64 | MS (ESI) m/z (M + H)⁺ = 423.1, ¹H NMR (400 MHz, DMSO-d₆) δ 9.39 (s, 1H), 9.04 (d, J = 2.5 Hz, 1H), 8.54 (dd, J = 8.8, 2.6 Hz, 1H), 7.01 (d, J = 8.8 Hz, 1H), 4.03 (s, 3H), 3.79 (t, J = 5.4 Hz, 2H), 3.45-3.41 (m, 2H), 1.80-1.70 (m, 4H), 1.63 (q, J = 5.6 Hz, 2H) |
| 81 | 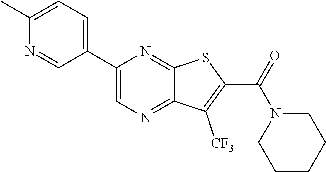 [SMILES: Cc1ccc(-c2cnc3c(C(F)(F)F)c(C(=O)N4CCCCC4)sc3n2)cn1] | Example 64 | MS (ESI) m/z (M + H)⁺ = 407.1, ¹H NMR (400 MHz, DMSO-d₆) δ 9.45 (s, 1H), 9.28 (d, J = 2.4 Hz, 1H), 8.58 (dd, J = 8.2, 2.4 Hz, 1H), 7.54 (d, J = 8.2 Hz, 1H), 3.80 (t, J = 5.3 Hz, 2H), 3.43 (t, J = 5.6 Hz, 2H), 2.66 (s, 3H), 1.80- 1.71 (m, 4H), 1.63 (d, J = 6.9 Hz, 2H) |
| 82 | 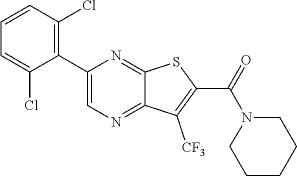 [SMILES: O=C(c1sc2nc(-c3c(Cl)cccc3Cl)cnc2c1C(F)(F)F)N1CCCCC1] | Example 64 | MS (ESI) m/z (M + H)⁺ = 460.1, ¹H NMR (400 MHz, DMSO-d₆) δ 8.93 (s, 1H), 7.65-7.62 (m, 2H), 7.56 (dd, J = 9.2, 6.8 Hz, 1H), 3.80 (t, J = 5.3 Hz, 2H), 3.47-3.42 (m, 2H), 1.75 (dt, J = 10.0, 4.5 Hz, 4H), 1.63 (p, J = 5.8 Hz, 2H) |
| 83 | 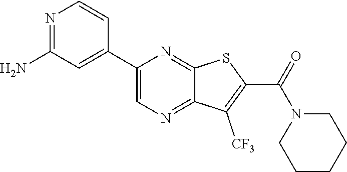 [SMILES: Nc1cc(-c2cnc3c(C(F)(F)F)c(C(=O)N4CCCCC4)sc3n2)ccn1] | Example 64 | MS (ESI) m/z (M + H)⁺ = 408.1, ¹H NMR (400 MHz, DMSO-d₆) δ 9.50 (s, 1H), 8.13 (d, J = 5.4 Hz, 1H), 7.35 (d, J = 5.2 Hz, 2H), 6.52 (s, 2H), 3.66 (t, J = 5.3 Hz, 2H), 3.36 (m, 2H), 1.65 (d, J = 7.1 Hz, 2H), 1.62-1.56 (m, 2H), 1.48 (d, J = 7.0 Hz, 2H) |
| 84 | 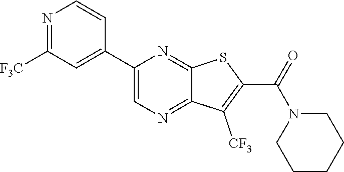 [SMILES: O=C(c1sc2nc(-c3ccnc(C(F)(F)F)c3)cnc2c1C(F)(F)F)N1CCCCC1] | Example 64 | MS (ESI) m/z (M + H)⁺ = 461.1, ¹H NMR (400 MHz, DMSO-d₆) δ 9.80 (s, 1H), 9.02 (d, J = 5.1 Hz, 1H), 8.68 (d, J = 1.6 Hz, 1H), 8.58 (dd, J = 5.1, 1.6 Hz, 1H), 3.67 (t, J = 5.3 Hz, 2H), 3.32 (m, 2H), 1.63 (ddd, J = 17.8, 9.8, 4.4 Hz, 4H), 1.49 (q, J = 5.6 Hz, 2H) |
| 85 | 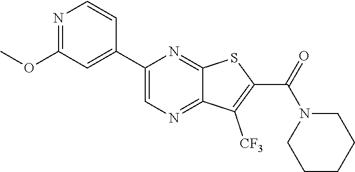 [SMILES: COc1cc(-c2cnc3c(C(F)(F)F)c(C(=O)N4CCCCC4)sc3n2)ccn1] | Example 64 | MS (ESI) m/z (M + H)⁺ = 423.1, ¹H NMR (400 MHz, DMSO-d₆) δ 9.47 (s, 1H), 8.35 (d, J = 5.4 Hz, 1H), 7.79 (dd, J = 5.4, 1.6 Hz, 1H), 7.64 (d, J = 1.5 Hz, 1H), 4.02 (s, 3H), 3.80 (t, J = 5.3 Hz, 2H), 3.43 (t, J = 5.6 Hz, 2H), 1.75 (tt, J = 10.0, 6.1 Hz, 4H), 1.63 (q, J = 5.7 Hz, 2H) |
| 86 | 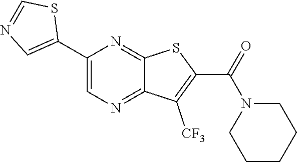 [SMILES: O=C(c1sc2nc(-c3cncs3)cnc2c1C(F)(F)F)N1CCCCC1] | Example 64 | MS (ESI) m/z (M + H)⁺ = 399.1, ¹H NMR (400 MHz, DMSO-d₆) δ 9.61 (s, 1H), 9.35 (d, J = 0.6 Hz, 1H), 8.95 (d, J = 0.7 Hz, 1H), 3.68-3.63 (m, 2H), 3.32 (m, 2H), 1.64 (d, J = 5.4 Hz, 2H), 1.61-1.56 (m, 2H), 1.47 (s, 2H) |
| 87 | 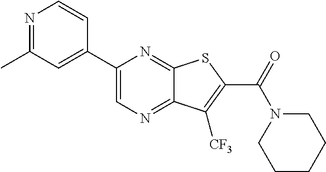 [SMILES: Cc1cc(-c2cnc3c(C(F)(F)F)c(C(=O)N4CCCCC4)sc3n2)ccn1] | Example 64 | MS (ESI) m/z (M + H)⁺ = 407.1, ¹H NMR (400 MHz, DMSO-d₆) δ 9.63 (s, 1H), 8.67-8.66 (d, J = 5.2 Hz, 1H), 8.10 (s, 1H), 8.02- 8.00 (dd, J = 5.3, 1.8 Hz, 1H), 3.68-3.65 (t, J = 5.4 Hz, 2H), 3.35-3.33(m, 2H), 2.61 (s, 3H), 1.66- 1.58 (m, 4H), 1.49-1.46 (t, J = 5.9 Hz, 2H) |
| 88 | 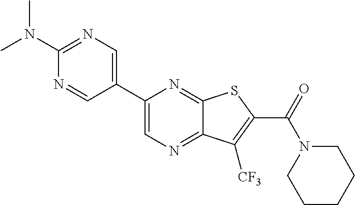 [SMILES: CN(C)c1ncc(-c2cnc3c(C(F)(F)F)c(C(=O)N4CCCCC4)sc3n2)cn1] | Example 64 | MS (ESI) m/z (M + H)⁺ = 437.1, ¹H NMR (400 MHz, DMSO-d₆) δ 9.47 (s, 1H), 9.20 (s, 2H), 3.66 (t, J = 5.4 Hz, 2H), 3.31 (m, 2H), 3.24 (s, 6H), 1.64 (d, J = 5.5 Hz, 2H), 1.59 (d, J = 4.7 Hz, 2H), 1.48 (s, 2H) |
| 89 | 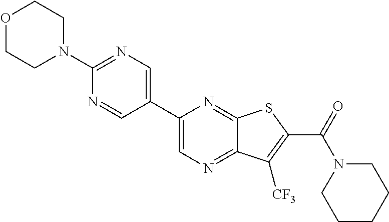 [SMILES: O=C(c1sc2nc(-c3cnc(N4CCOCC4)nc3)cnc2c1C(F)(F)F)N1CCCCC1] | Example 64 | MS (ESI) m/z (M + H)⁺ = 479.1, ¹H NMR (400 MHz, DMSO-d₆) δ 9.49 (s, 1H), 9.22 (s, 2H), 3.87-3.84 (t, J = 4.8 Hz, 4H), 3.71- 3.69 (dd, J = 5.6, 4.1 Hz, 4H), 3.67-3.64 (d, J = 5.7 Hz, 2H), 3.34-3.31 (d, J = 5.9 Hz, 2H), 1.65-1.58 (dd, J = 22.1, 5.4 Hz, 4H), 1.481-1.47 (m, 2H) |
| 90 | 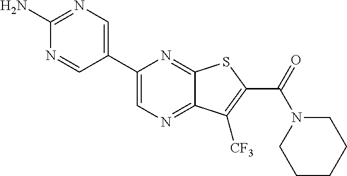 [SMILES: Nc1ncc(-c2cnc3c(C(F)(F)F)c(C(=O)N4CCCCC4)sc3n2)cn1] | Example 64 | MS (ESI) m/z (M + H)⁺ = 410.1, ¹H NMR (400 MHz, DMSO-d₆) δ 9.46 (s, 1H), 9.11 (s, 2H), 7.36 (s, 2H), 3.65 (t, J = 5.3 Hz, 2H), 3.35-3.33(m, 2H), 1.69-1.62 (m, 2H), 1.61- 1.56 (m, 2H), 1.47 (t, J = 5.6 Hz, 2H) |
| 91 | 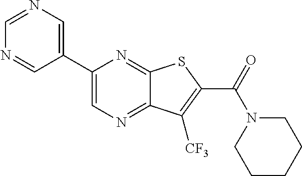 [SMILES: O=C(c1sc2nc(-c3cncnc3)cnc2c1C(F)(F)F)N1CCCCC1] | Example 64 | MS (ESI) m/z (M + H)⁺ = 394.1, ¹H NMR (400 MHz, DMSO-d₆) δ 9.69 (s, 1H), 9.62 (s, 2H), 9.38 (s, 1H), 3.67 (t, J = 5.3 Hz, 2H), 3.32 (d, J = 5.2 Hz, 2H), 1.69-1.63 (m, 2H), 1.62- 1.56 (m, 2H), 1.49 (d, J = 6.2 Hz, 2H) |
| 92 | 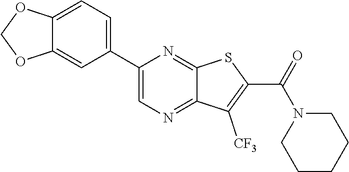 [SMILES: O=C(c1sc2nc(-c3ccc4c(c3)OCO4)cnc2c1C(F)(F)F)N1CCCCC1] | Example 64 | MS (ESI) m/z (M + H)⁺ = 436.1, ¹H NMR (400 MHz, DMSO-d₆) δ 9.50 (s, 1H), 7.86 (dd, J = 8.2, 1.9 Hz, 1H), 7.83 (d, J = 1.8 Hz, 1H), 7.14 (d, J = 8.2 Hz, 1H), 6.16 (s, 2H), 3.66 (t, J = 5.3 Hz, 2H), 3.32 (d, J = 2.8 Hz, 2H), 1.64- 1.55 (m, 4H), 1.48 (s, 2H) |
| 93 | 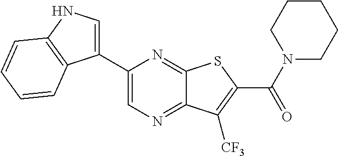 [SMILES: O=C(c1sc2nc(-c3c[nH]c4ccccc34)cnc2c1C(F)(F)F)N1CCCCC1] | Example 64 | MS (ESI) m/z (M + H)⁺ = 431.1, ¹H NMR (400 MHz, DMSO-d₆) δ 11.99 (s, 1H), 9.46 (d, J = 2.0 Hz, 1H), 8.58-8.51 (m, 2H), 7.53 (d, J = 7.5 Hz, 1H), 7.25 (t, J = 6.7 Hz, 2H), 3.66 (t, J = 5.2 Hz, 2H), 3.36 (m, 2H), 1.65 (d, J = 6.2 Hz, 2H), 1.62-1.56 (m, 2H), 1.53-1.45 (m, 2H) |
| 94 | 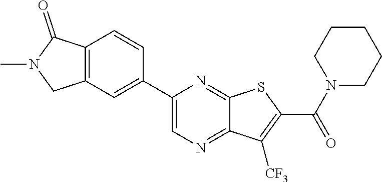 [SMILES: CN1Cc2cc(-c3cnc4c(C(F)(F)F)c(C(=O)N5CCCCC5)sc4n3)ccc2C1=O] | Example 64 | MS (ESI) m/z (M + H)⁺ = 461.1, ¹H NMR (400 MHz, DMSO-d₆) δ 9.63 (d, J = 1.5 Hz, 1H), 8.49 (s, 1H), 8.38 (d, J = 8.1 Hz, 1H), 7.86 (d, J = 8.2 Hz, 1H), 4.60 (s, 2H), 3.67 (t, J = 5.1 Hz, 2H), 3.36 (d, J = 5.1 Hz, 2H), 3.13 (s, 3H), 1.69-1.57 (m, 4H), 1.49 (d, J = 6.1 Hz, 2H) |
| 95 | 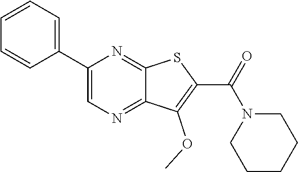 [SMILES: COc1c(C(=O)N2CCCCC2)sc2nc(-c3ccccc3)cnc12] | Example 1 | MS (ESI) m/z (M + H)⁺ = 354.1, ¹H NMR (400 MHz, DMSO-d₆) δ 9.41 (m, 1H), 8.25-8.23 (m, 2H), 7.60-7.537 (m, 3H), 4.24 (s, 3H), 3.63-3.49 (m, 4H), 1.68-1.57 (m, 6H). |
| 96 | 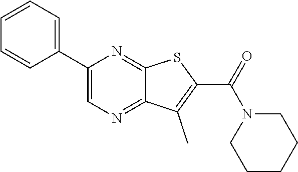 [SMILES: Cc1c(C(=O)N2CCCCC2)sc2nc(-c3ccccc3)cnc12] | Example 1 | MS (ESI) m/z (M + H)⁺ = 338.1, ¹H NMR (400 MHz, DMSO-d₆) δ 9.43 (s, 1H), 8.25-8.23 (m, 2H), 7.61-7.55(m, 3H), 3.60-3.33 (m, 4H), 2.42 (s, 3H), 1.65-1.56 (m, 6H). |
| 97 | 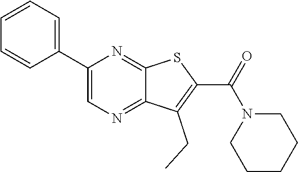 [SMILES: CCc1c(C(=O)N2CCCCC2)sc2nc(-c3ccccc3)cnc12] | Example 1 | MS (ESI) m/z (M + H)⁺ = 352.1, ¹H NMR (400 MHz, DMSO-d₆) δ 9.41 (s, 1H), 8.24-8.21 (dd, J = 8.2, 1.6 Hz, 2H), 7.60-7.51 (m, 3H), 3.64-3.42 (m, 4H), 2.90-2.85 (q, J = 7.5 Hz, 2H), 1.66-1.54 (m, 6H), 1.28-1.25 (t, J = 7.5 Hz, 3H). |
| 98 | 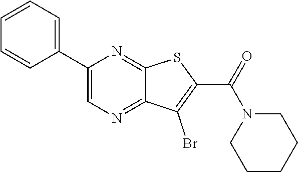 [SMILES: O=C(c1sc2nc(-c3ccccc3)cnc2c1Br)N1CCCCC1] | Example 1 | MS (ESI) m/z (M + H)⁺ = 402.0, ¹H NMR (400 MHz, DMSO-d₆) δ 9.52 (s, 1H), 8.28-8.26 (m, 2H), 7.63-7.55 (m, 3H), 3.68 (m, 2H), 3.40 (m, 2H), 1.64-1.58 (m, 6H). |
| 99 | 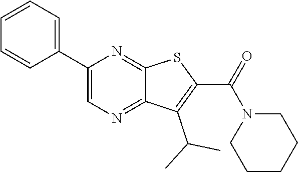 [SMILES: CC(C)c1c(C(=O)N2CCCCC2)sc2nc(-c3ccccc3)cnc12] | Example 1 | MS (ESI) m/z (M + H)⁺ = 366.1, ¹H NMR (400 MHz, DMSO-d₆) δ 9.42 (s, 1H), 8.24-8.21 (m, 2H), 7.60-7.54 (m, 3H), 3.66 (m, 2H), 3.37 (m, 2H), 3.36-3.29 (m, 1H), 1.64-1.50 (m, 6H), 1.45-1.44 (d, J = 7.0 Hz, 6H). |
| 100 | 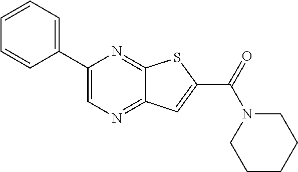 [SMILES: O=C(c1cc2ncc(-c3ccccc3)nc2s1)N1CCCCC1] | Example 1 | MS (ESI) m/z (M + H)⁺ = 324.1, ¹H NMR (400 MHz, DMSO-d₆) δ 9.45 (s, 1H), 8.27-8.25(m, 2H), 7.86 (s, 1H), 7.61-7.55(m, 3H), 3.67-3.64 (t, J = 5.4 Hz, 4H), 1.69-1.58 (m, 6H). |
| 101 | 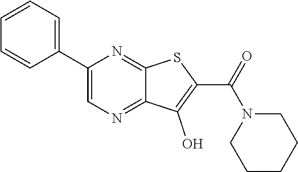 [SMILES: O=C(c1sc2nc(-c3ccccc3)cnc2c1O)N1CCCCC1] | Example 1 | MS (ESI) m/z (M + H)⁺ = 340.1, ¹H NMR (400 MHz, DMSO-d₆) δ 9.39 (s, 1H), 8.27-8.24 (m, 2H), 7.61-7.55 (m, 3H), 3.59-3.56 (t, J = 5.3 Hz, 4H), 1.64-1.57 (m, 6H). |

### Biological Tests

### Test Example 1: Detection of Activity of 15-PGDH Enzyme

### 1. Experimental Materials:

| Reagents/Materials/Instruments | Manufacturer | Item No. / Model No. |
|---|---|---|
| 15-PGDH | Sino Biological Inc. | 11205-H08E |
| β-NAD | Sigma-Aldrich Corporation | N6522 |
| PGF2α | MedChemExpress LLC | HY-12956A |
| DMSO | Sigma-Aldrich Corporation | D8418 |
| 384-well plate | Corning United States Corporation | 4513 |
| Tween 20 | Shanghai Macklin Biochemical | T818927 |
| | Technology Co., Ltd. | |
| Tris-HCl | Shanghai Beyotime Biotech Inc. | ST774 |
| Multifunctional microplate | BMG LABTECH Corporation | PHERAstar ® FSX |
| reader | | |

### 2. Experimental Method:

a. A solution of pH 7.5 containing 50 mM Tris-HCl, 0.01% Tween 20 was prepared with ultrapure water as a reaction buffer;
b. A 10 mM mother liquor of the compound to be tested was prepared with DMSO, and then the reaction buffer was used to dilute the mother liquor of the compound to be tested to obtain solution 1 of the compound to be tested at a concentration of 40,000 nM, and then the solution 1 of the compound to be tested was serially diluted into solutions 2-9 (or 2-12) of the compound to be tested at 9 (or 11) concentrations with a gradient difference of three-fold. 5 μL of each concentration of solutions of the compound to be tested was respectively taken and added into a 384-well plate as test wells;
c. 5 μL of the reaction buffer was then added to the blank wells of the 384-well plate as positive control and blank control wells, respectively;
d. The reaction buffer was used to prepare a 15-PGDH protein solution at a concentration of 5 ng/μL, 5 μL of the 15-PGDH protein solution was taken and added to the test wells and positive control wells, and meanwhile 5 μL of the reaction buffer was added to the blank control wells, then the plate was centrifuged at 2000 rpm for 30 seconds;
e. The reaction buffer was used to prepare 5 mM $3-NAD and 2 mM PGF2α, respectively, which were mixed at 1:1 by volume to obtain a substrate mixture, 10 μL of the substrate mixture was taken and added to the test wells, positive control wells and blank control wells to start the reaction;
f. The fluorescence signal value (Ex/Em=340/450) of each well was detected continuously by using a multifunctional microplate reader.

### 3. Data Analysis:

a) Continuous fluorescence signal values were analyzed by using the “kinetic calculations-slope calculation method” in the PHERAstar Data analysis software to obtain the slope of each test well;
b) The inhibition rate % was calculated by using the following formula: inhibition rate %=[1−(slope of test well-signal value of positive control well)/(signal value of blank control well-average signal value of positive control well)]×100%.
c) Calculation of IC50 and plotting of inhibition rate-dose curves: IC50 values were calculated by fitting compound concentrations and corresponding inhibition rates with a nonlinear regression (dose response-variable slope) via using GraphPad Prism 6.0. The formula was shown below:

Y=Bottom+(Top−Bottom)/(1+10\hat{ }((Log IC50−X)*HillSlope)), wherein X is a log value of a concentration of the compound, and Y is inhibition rate %.

### 4. Experimental Results:

Inhibitory activities of some compounds in the present application against the 15-PGDH enzyme are as follows:

| Example No. | IC₅₀ (nM) |
|---|---|
| Example 1 | A |
| Example 2 | B |
| Example 3 | A |
| Example 4 | B |
| Example 5 | A |
| Example 6 | B |
| Example 7 | B |
| Example 8 | B |
| Example 9 | B |
| Example 10 | B |
| Example 11 | C |
| Example 16 | C |
| Example 17 | B |
| Example 18 | A |
| Example 19 | B |
| Example 20 | B |
| Example 21 | B |
| Example 12 | A |
| Example 13 | B |
| Example 14 | B |
| Example 22 | A |
| Example 23 | C |
| Example 24 | A |
| Example 25 | C |
| Example 26 | A |
| Example 27 | B |
| Example 28 | B |
| Example 29 | B |
| Example 30 | B |
| Example 31 | A |
| Example 32 | A |
| Example 33 | C |
| Example 34 | C |
| Example 43 | C |
| Example 44 | C |
| Example 45 | B |
| Example 47 | B |
| Example 48 | B |
| Example 50 | C |
| Example 51 | C |
| Example 55 | C |
| Example 56 | B |
| Example 57 | B |
| Example 58 | B |
| Example 59 | B |
| Example 60 | B |
| Example 61 | B |
| Example 62 | B |
| Example 63 | B |
| Example 64 | B |
| Example 65 | B |
| Example 67 | C |
| Example 68 | B |
| Example 69 | A |
| Example 70 | C |
| Example 71 | B |
| Example 72 | B |
| Example 74 | A |
| Example 75 | C |
| Example 76 | C |
| Example 77 | B |
| Example 78 | C |
| Example 79 | B |
| Example 80 | B |
| Example 83 | B |
| Example 85 | B |
| Example 86 | B |
| Example 87 | A |
| Example 88 | C |
| Example 89 | C |
| Example 90 | A |
| Example 91 | B |
| Example 94 | B |
| Example 95 | B |
| Example 96 | C |
| Example 97 | B |
| Example 98 | A |
| Example 99 | C |
| Example 100 | C |
| Example 101 | E |

In the table, “/” represents no detection; “A” represents that IC₅₀ of the inhibitory activity against 15-PGDH enzyme is in a range of less than 3 nM; “B” represents that IC₅₀ of the inhibitory activity against 15-PGDH enzyme is in a range of equal to or greater than 3 nM and less than 10 nM; “C” represents that IC₅₀ of the inhibitory activity against 15-PGDH enzyme is in a range of equal to or greater than 10 nM and less than 20 nM; “D” represents that IC₅₀ of the inhibitory activity against 15-PGDH enzyme is in a range of equal to or greater than 20 nM and equal to or less than 50 nM; and “E” represents that IC₅₀ of the inhibitory activity against 15-PGDH enzyme is in a range of equal to or greater than 50 nM and equal to or less than 100 nM

It was found via the tests that the inhibitory activity of the compounds of the present application against 15-PGDH enzyme is in a range of equal to or less than 100 nM. IC₅₀ of the inhibitory activity of some compounds in the present application against 15-PGDH enzyme is in a range of less than 50 nM; IC₅₀ of the inhibitory activity of some compounds of the present application against 15-PGDH enzyme is in a range of less than 20 nM; IC₅₀ of the inhibitory activity of some compounds in the present application against 15-PGDH enzyme is in a range of less than 10 nM; and IC₅₀ of the inhibitory activity of some compounds of the present application against 15-PGDH enzyme is in a range of less than 3 nM.

The results showed that the compounds of the present application, especially some compounds in examples, can exhibit strong inhibitory activity against 15-PGDH enzyme.

### Test Example 2: Assay of Intracellular PGE2 Up-Regulatory Activity

### 1. Experimental Materials:

| Reagents/Materials/Instruments | Manufacturer | Item No. |
|---|---|---|
| F12k Kaighn's Modification culture | Hyclone Laboratories, Inc | SH3052601/AG29722854 |
| medium | | |
| TRYPSIN | Hyclone Laboratories, Inc | J190002 |
| Fetal Bovine Serum | PAN-Biotech | ST-30-3302 |
| Penicillin-Streptomycin | Hyclone Laboratories, Inc | J190007 |
| DMSO | Sigma-Aldrich Corporation | D8418 |
| A549 cells | Nanjing Cobioer Biosciences | CBP60084 |
| | Co., Ltd. | |
| Prostaglandin E2 Kit | PerkinElmer Corporation | 62P2APEG |
| 24-well plate | Corning United States | 3337 |
| | Corporation | |
| 384-well plate | Corning United States | 3570 |
| | Corporation | |
| Multifunctional microplate reader | BMG LABTECH Corporation | PHERAstar ® FSX |
| CO₂ cell incubator | Thermo Fisher Scientific | RI-250 |
| | Corporation | |
| Microscope | Thermo Fisher Scientific | DMI1 |
| | Corporation | |

### 2. Experimental Method:

a) A549 cells were inoculated in the 24-well plate, and after cell adhesion, IL-1β was added thereto for 16 h of stimulation to induce COX2 expression and PGE2 production;
b) A solution of the compound to be tested was prepared with the culture medium and gradiently diluted to 3 concentrations of 5 nM, 50 nM and 500 nM, and meanwhile the positive control group (only IL-1β was added to the cells for stimulation) and negative control group (only cells were added in the wells without any treatment) were set up; the cell supernatants were collected after 8 h of action, in which the positive control group was induced by IL-1β without treatment of the compounds, and the negative control group was neither stimulated by IL-1β, nor treated with the compounds;
c) The PGE2 content of the samples was determined by Prostaglandin E2 Kit, and the fluorescence signal was detected by a multifunctional microplate reader (Ex/Em=337/620, 337/665).

### 3. Data Analysis:

a) A standard curve was plotted with the PGE2 standard in the Prostaglandin E2 Kit, and the PGE2 concentration was calculated by substituting with the fluorescence signal of the sample.
b) The PGE2 up-regulation rate % was calculated by using the following formula:

PGE2 up-regulation rate %=(PGE2 concentration of sample group/PGE2 concentration of positive control group)×100%.

### 4. Experimental Results

The compounds in the examples of the present application are able to achieve a PGE2 up-regulation rate of greater than 100% in A549 cells. The compounds of the present application have good intracellular PGE2 up-regulatory activity.

For the purpose of describing and disclosing, all patents, patent applications and other established publications are expressly incorporated herein by reference. These publications are provided solely for their disclosure prior to the filing date of this application. All statements regarding the dates of these documents or the representation of the contents of these documents are based on the information available to the applicants and do not constitute any admission as to the correctness of the dates of these documents or the contents of these documents. Moreover, any reference to these publications herein does not constitute an admission that the publications form part of the common general knowledge in the art in any country.

Those skilled in the art will recognize that the scope of the present application is not limited to the various specific embodiments and examples described above, but is capable of making various modifications, substitutions, or recombinations without departing from the spirit of the present application, and that these adjusted technical solutions fall within the protection scope of the present application.

1. A compound represented by formula (I), a stereoisomer,
tautomer or mixture form thereof, or a pharmaceutically acceptable salt thereof, or a solvate thereof, or a prodrug thereof: 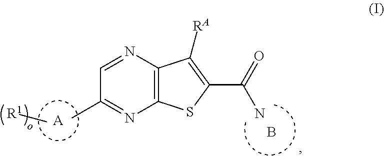
ring A an aromatic ring, an aromatic heterocycle, an unsaturated aliphatic heterocycle, a fused ring consisted of an aromatic ring and an unsaturated aliphatic heterocycle, and a fused ring consisted of an aromatic heterocycle o an unsaturated aliphatic heterocycle;
ring B is a 3-12 membered saturated aliphatic heterocycle;
R^{A} is hydrogen, deuterium, tritium, hydroxy, halogen, cyano, C₁-C₆ alkyl, C₁-C₆ alkoxy, C₁-C₆ haloalkyl, or 3-8 membered cycloalkyl;
o is 0, 1, 2, or 3;
R¹ is each independently selected from the group consisting of deuterium, tritium, nitro, hydroxy, mercapto, halogen, cyano, =O, imino, an amine group, an ester group, an aldehyde group, carboxyl, amido, C₁-C₆ alkyl, C₁-C₆ halogenated alkyl, C₁-C₆ alkoxy, C₁-C₆ halogenated alkoxy, 3-8 membered cycloalkyl, and 3-8 membered saturated aliphatic heterocyclyl;
wherein the aromatic heterocycle, the saturated aliphatic heterocycle, the unsaturated aliphatic heterocycle, the aliphatic heterocyclyl, and the fused ring each independently comprise 1-3 heteroatoms which are independently selected from the group consisting of N, O, and S, and ring B comprises at least 1 nitrogen atom;
the ring B and R¹ are optionally substituted by one or more independently selected from the group consisting of deuterium, tritium, nitro, hydroxy, an aldehyde group, an amine group, imino, halogen, cyano, an ester group, carboxyl, amido, =O, C₁-C₆ alkyl, C₁-C₆ alkoxy, 3-8 membered cycloalkyl, 6-10 membered aryl, 5-10 membered aliphatic heterocyclyl, and 5-10 membered heteroaryl.

2-15. (canceled)

16. The compound, the stereoisomer, tautomer or mixture form thereof, or the pharmaceutically acceptable salt thereof, or the solvate thereof, or the prodrug thereof according to claim 1, wherein the ring B is a monocyclic ring or a bicyclic ring.

17. The compound, the stereoisomer, tautomer or mixture form thereof, or the pharmaceutically acceptable salt thereof, or the solvate thereof, or the prodrug thereof according to claim 1, wherein
ring B is 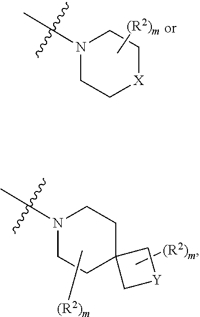
wherein X is a covalent bond, O, S, NH, (CH₂)ₙ, or SO₂; Y is a covalent bond, S, NH, (CH₂)ₙ, or SO₂; m is 0, 1, 2, or 3; R² is each independently selected from the group consisting of deuterium, tritium, nitro, hydroxy, mercapto, cyano, halogen, an amine group, an ester group, an aldehyde group, carboxyl, amido, C₁-C₆ alkyl, C₁-C₆ haloalkyl, C₁-C₆ alkoxy, 3-8 membered cycloalkyl, 6-10 membered aryl, and 5-10 membered heteroaryl; n is 0, 1, 2, or 3.

18. The compound, the stereoisomer, tautomer or mixture form thereof, or the pharmaceutically acceptable salt thereof, or the solvate thereof, or the prodrug thereof according to claim 17, wherein the ring B is

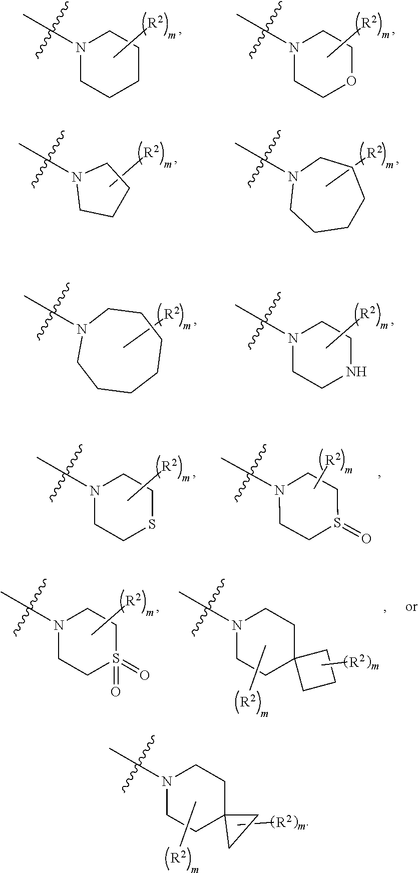

19. The compound, the stereoisomer, tautomer or mixture form thereof, or the pharmaceutically acceptable salt thereof, or the solvate thereof, or the prodrug thereof according to claim 17, wherein the R² is each independently selected from the group consisting of deuterium, tritium, nitro, hydroxy, mercapto, cyano, fluoro, chloro, bromo, an amine group, an ester group, an aldehyde group, carboxyl, amido, methyl, ethyl, n-propyl, isopropyl, methoxy, ethoxy, n-propoxy, isopropoxy, trifluoromethyl, trifluoroethyl, trichloromethyl, trichloroethyl, cyclobutyl, cyclopropyl, phenyl, and pyridyl.

20. The compound, the stereoisomer, tautomer or mixture form thereof, or the pharmaceutically acceptable salt thereof, or the solvate thereof, or the prodrug thereof according to claim 1, wherein the R^{A} is hydrogen, deuterium, tritium, hydroxy, halogen, cyano, cyclopropyl, cyclobutyl, methyl, ethyl, methoxy, ethoxy, or trifluoromethyl;
or, the R^{A} is hydrogen, deuterium, tritium, hydroxy, fluoro, chloro, bromo, cyano, cyclopropyl, cyclobutyl, methyl, ethyl, propyl, butyl, pentyl, methoxy, ethoxy, propoxy, C₁-C₃ fluoroalkyl, or C₁-C₃ bromoalkyl.

21. The compound, the stereoisomer, tautomer or mixture form thereof, or the pharmaceutically acceptable salt thereof, or the solvate thereof, or the prodrug thereof according to claim 1, wherein the compound has a structure represented by formula (II), 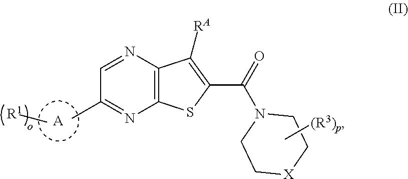
wherein X is a covalent bond, S, CH₂, (CH₂)₂, or (CH₂)₃; R³ is each independently selected from the group consisting of deuterium, tritium, nitro, hydroxy, mercapto, cyano, halogen, an amine group, an ester group, an aldehyde group, carboxyl, amido, C₁-C₆ alkyl, C₁-C₆ haloalkyl, C₁-C₆ alkoxy, C₃-C₈ cycloalkyl, 6-10 membered aryl, and 5-10 membered heteroaryl; p is 0 or 1.

22. The compound, the stereoisomer, tautomer or mixture form thereof, or the pharmaceutically acceptable salt thereof, or the solvate thereof, or the prodrug thereof according to claim 21, wherein the p is 0.

23. The compound, the stereoisomer, tautomer or mixture form thereof, or the pharmaceutically acceptable salt thereof, or the solvate thereof, or the prodrug thereof according to claim 21, wherein the R³ is each independently selected from the group consisting of deuterium, tritium, nitro, hydroxy, mercapto, cyano, fluoro, chloro, bromo, an amine group, an ester group, an aldehyde group, carboxyl, amido, methyl, ethyl, n-propyl, isopropyl, methoxy, ethoxy, n-propoxy, isopropoxy, trifluoromethyl, trifluoroethyl, trichloromethyl, trichloroethyl, cyclobutyl, cyclopropyl, phenyl, and pyridyl; the p is 0, and the X is CH₂.

24. The compound, the stereoisomer, tautomer or mixture form thereof, or the pharmaceutically acceptable salt thereof, or the solvate thereof, or the prodrug thereof according to claim 21, wherein the R^{A} is hydrogen, deuterium, tritium, hydroxy, halogen, cyano, cyclopropyl, cyclobutyl, methyl, ethyl, methoxy, ethoxy, or trifluoromethyl;
or, the R^{A} is hydrogen, deuterium, tritium, hydroxy, fluoro, chloro, bromo, cyano, cyclopropyl, cyclobutyl, methyl, ethyl, propyl, butyl, pentyl, methoxy, ethoxy, propoxy, C₁-C₃ fluoroalkyl, or C₁-C₃ bromoalkyl.

25. The compound, the stereoisomer, tautomer or mixture form thereof, or the pharmaceutically acceptable salt thereof, or the solvate thereof, or the prodrug thereof according to claim 24, wherein the R^{A} is hydrogen, deuterium, tritium, cyano, cyclopropyl, trifluoromethyl, halogen, or methyl; or the R^{A} is hydrogen, deuterium, tritium, cyano, cyclopropyl, cyclobutyl, trifluoromethyl, fluoro, chloro, bromo, methyl, ethyl, propyl, butyl, pentyl, methoxy, or ethoxy.

26. The compound, the stereoisomer, tautomer or mixture form thereof, or the pharmaceutically acceptable salt thereof, or the solvate thereof, or the prodrug thereof according to claim 1, wherein ring A is a 6-10 membered aromatic ring, a 5-10 membered aromatic heterocycle, a 3-8 membered unsaturated aliphatic heterocycle, a 7-12 membered fused ring consisted of an aromatic ring and an unsaturated aliphatic heterocycle, or a 7-12 membered fused ring consisted of an aromatic heterocycle and an unsaturated aliphatic heterocycle;
the aromatic ring and the aromatic heterocycle are a monocyclic ring or a bicyclic ring, the unsaturated aliphatic heterocycle is a monocyclic ring, the fused ring is a bicyclic ring, and the aromatic heterocycle, the unsaturated aliphatic heterocycle, and the fused ring each independently comprise 1-3 heteroatoms which are independently selected from the group consisting of N, O, and S.

27. The compound, the stereoisomer, tautomer or mixture form thereof, or the pharmaceutically acceptable salt thereof, or the solvate thereof, or the prodrug thereof according to claim 26, wherein ring A is

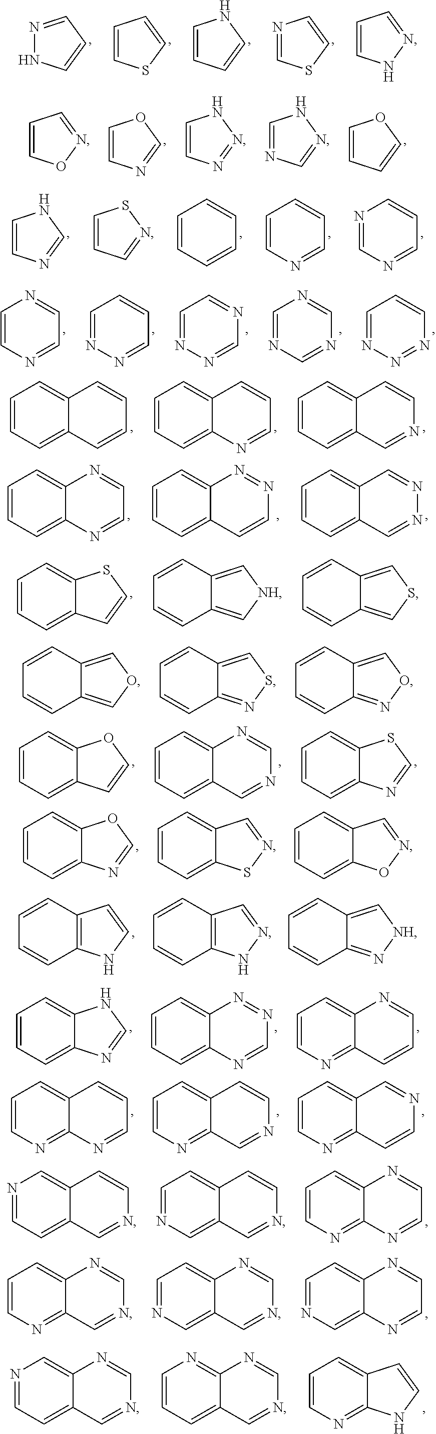

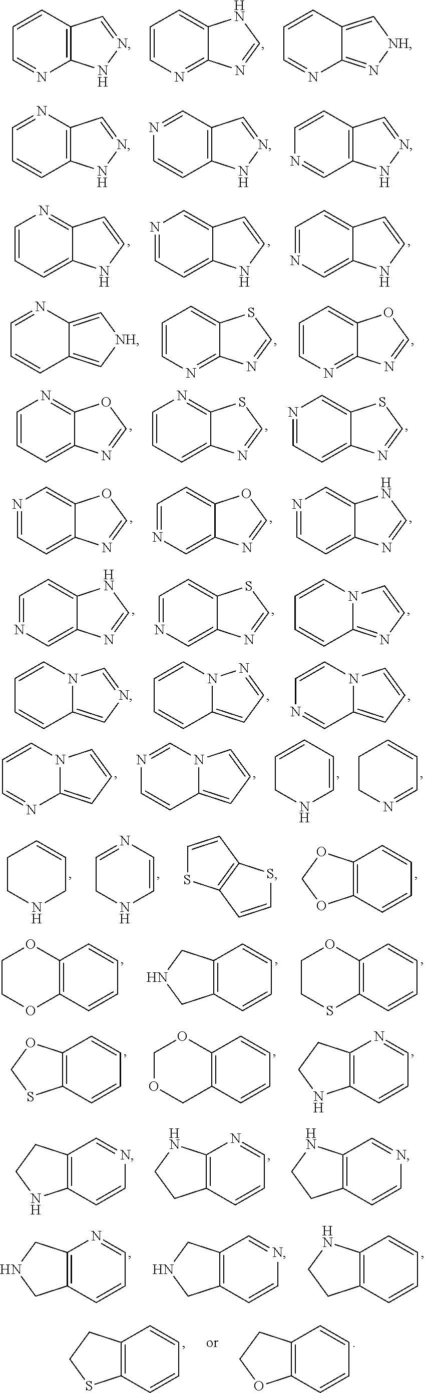

28. The compound, the stereoisomer, tautomer or mixture form thereof, or the pharmaceutically acceptable salt thereof, or the solvate thereof, or the prodrug thereof according to claim 1, wherein
R¹ is each independently selected from the group consisting of deuterium, tritium, nitro, hydroxy, mercapto, halogen, cyano, =O, imino, an amine group, an ester group, an aldehyde group, carboxyl, amido, cyclopropyl, cyclopropylmethyl, cyclobutyl, cyclohexyl, cyclopentyl, methyl, trifluoromethyl, ethyl, isopropyl, n-butyl, isobutyl, tert-butyl, n-pentyl, isopentyl, tert-pentyl, n-hexyl, morpholinyl, thiomorpholinyl, piperidinyl, piperazinyl, oxazolidinyl, isoxazolidinyl, thiazolidinyl, isothiazolidinyl, dioxolanyl, dioxanyl, methoxy, ethoxy, n-propoxy, isopropoxy, cyclopropoxy, cyclopropylmethoxy, n-butoxy, isobutoxy, tert-butoxy, n-pentyloxy, isopentyloxy, tert-pentyloxy, and n-hexyloxy, wherein the R¹ is optionally substituted by one or more independently selected from the group consisting of deuterium, tritium, nitro, hydroxy, —NH₂, mercapto, halogen, cyano, an ester group, carboxyl, amido, =O, =NH, C₁-C₆ alkyl, C₁-C₆ halogenated alkyl, C₁-C₆ alkoxy, C₁-C₆ halogenated alkoxy, 3-8 membered cycloalkyl, 6-10 membered aryl, 5-10 membered aliphatic heterocyclyl, and 5-10 membered heteroaryl.

29. The compound, the stereoisomer, tautomer or mixture form thereof, or the pharmaceutically acceptable salt thereof, or the solvate thereof, or the prodrug thereof according to claim 1, wherein
the ring A is a 6-10 membered aromatic ring, a 5-10 membered aromatic heterocycle comprising 1-2 heteroatoms selected from the group consisting of N, O, and S, a 4-7 membered unsaturated aliphatic heterocycle comprising 1-2 heteroatoms selected from the group consisting of N, O, and S, a 8-12 membered fused ring consisted of an aromatic ring and an unsaturated aliphatic heterocycle, or a 8-12 membered fused ring consisted of an aromatic heterocycle and an unsaturated aliphatic heterocycle, and the fused ring comprises 1-2 heteroatoms selected from the group consisting of N, O, and S;
the ring B is a 5-10 membered saturated aliphatic heterocycle comprising at least 1 nitrogen atom;
m is 0, 1, 2, or 3; R² is each independently selected from the group consisting of deuterium, tritium, nitro, hydroxy, mercapto, cyano, halogen, an amine group, an ester group, an aldehyde group, carboxyl, amido, C₁-C₆ alkyl, C₁-C₆ haloalkyl, C₁-C₆ alkoxy, 3-8 membered cycloalkyl, 6-10 membered aryl, and 5-10 membered heteroaryl;
the R^{A} is hydrogen, deuterium, tritium, hydroxy, fluoro, chloro, bromo, cyano, C₁-C₅ alkyl, C₁-C₅ alkoxy, C₁-C₅ haloalkyl, or 3-6 membered cycloalkyl;
o is 0, 1, or 2, and R¹ is each independently selected from the group consisting of deuterium, tritium, hydroxy, halogen, cyano, =O, imino, an amine group, C₁-C₆ alkyl, C₁-C₆ halogenated alkyl, C₁-C₆ alkoxy, 4-8 membered cycloalkyl, and 4-8 membered saturated aliphatic heterocyclyl, and the R¹ is optionally substituted by C₁-C₆ alkyl.

30. The compound, the stereoisomer, tautomer or mixture form thereof, or the pharmaceutically acceptable salt thereof, or the solvate thereof, or the prodrug thereof according to claim 1, wherein
the ring A is 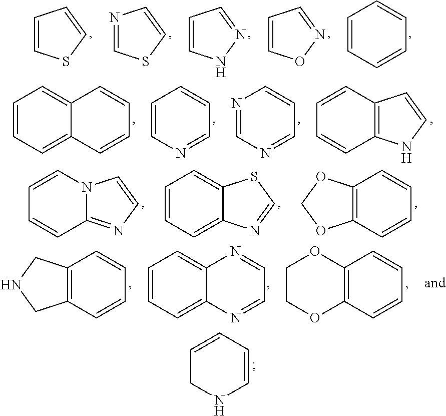
the ring B is 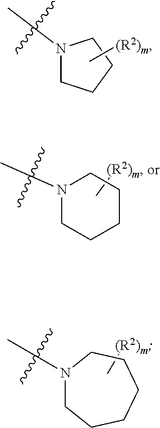
m is 0 or 1;
R² is each independently selected from the group consisting of deuterium, tritium, hydroxy, fluoro, chloro, bromo, an amine group, methyl, and ethyl;
the R^{A} is hydrogen, deuterium, tritium, fluoro, chloro, bromo, cyano, cyclopropyl, cyclobutyl, methyl, ethyl, propyl, butyl, methoxy, ethoxy, or C₁-C₃ fluoroalkyl;
o is 0, 1, or 2, and R¹ is each independently selected from the group consisting of fluoro, chloro, bromo, cyano, =O, an amine group, methyl, ethyl, propyl, methoxy, ethoxy, propoxy, morpholinyl, thiomorpholinyl, piperidinyl, and piperazinyl, wherein the amine group is optionally substituted by methyl, ethyl, or propyl.

31. The compound, the stereoisomer, tautomer or mixture form thereof, or the pharmaceutically acceptable salt thereof, or the solvate thereof, or the prodrug thereof according to claim 1, wherein the compound is selected from the group consisting of the following compounds:

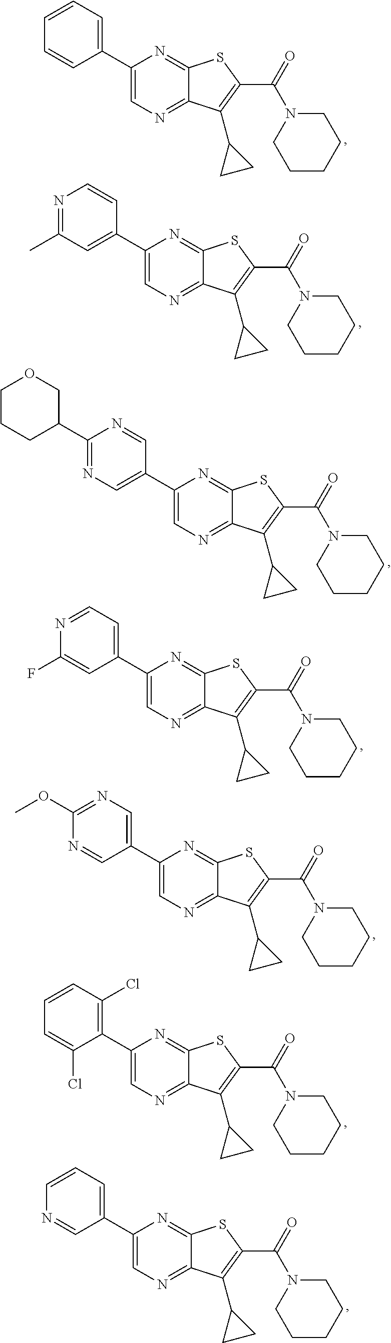

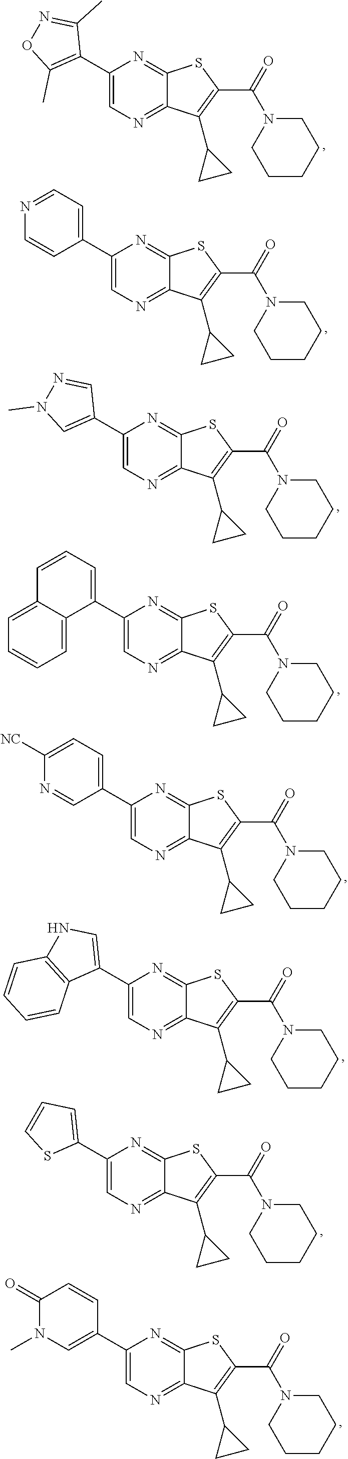

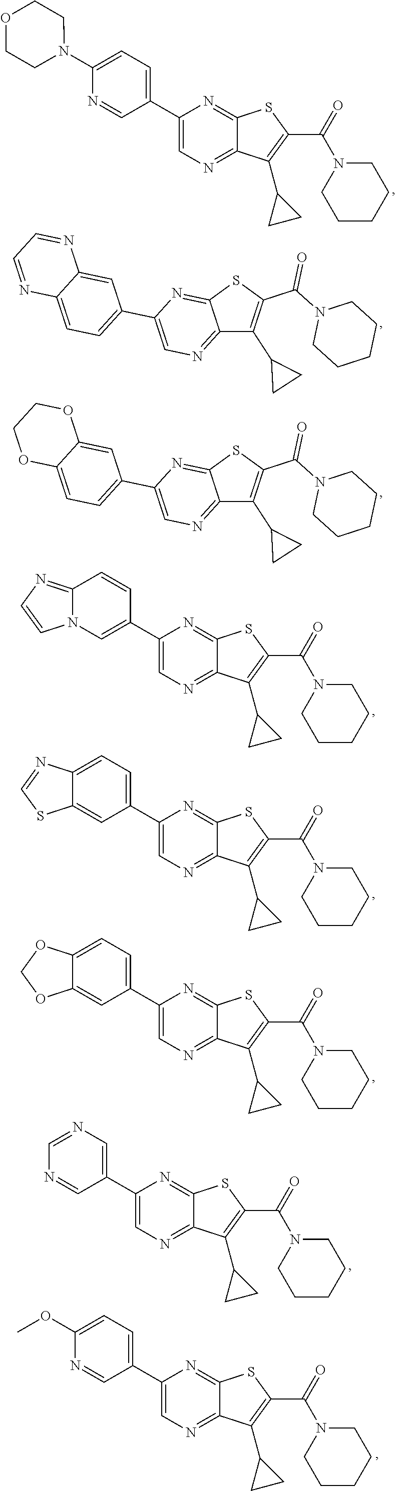

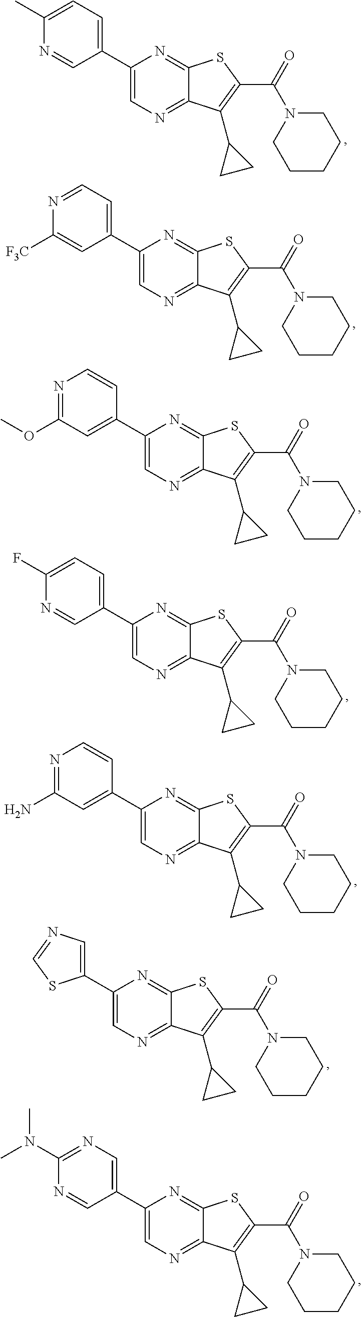

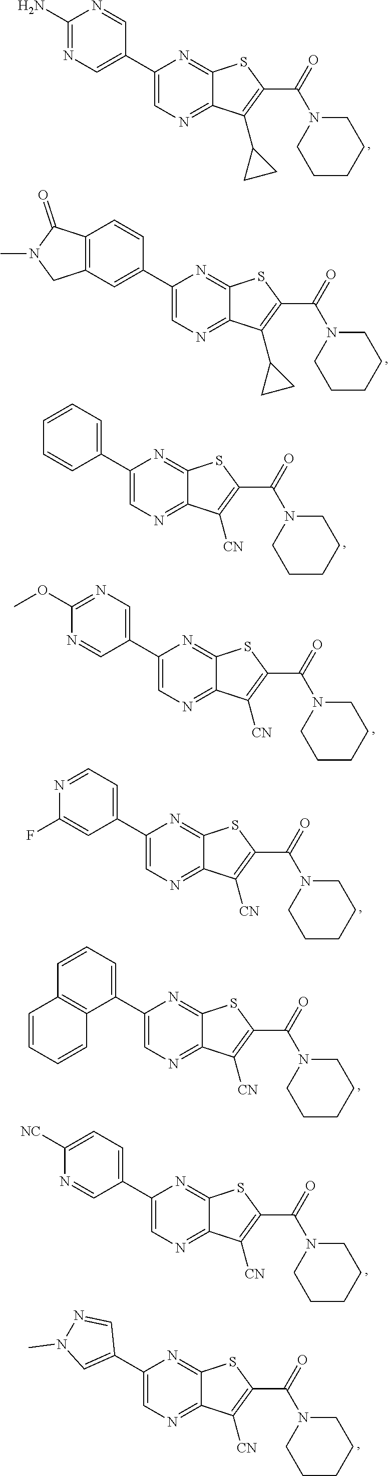

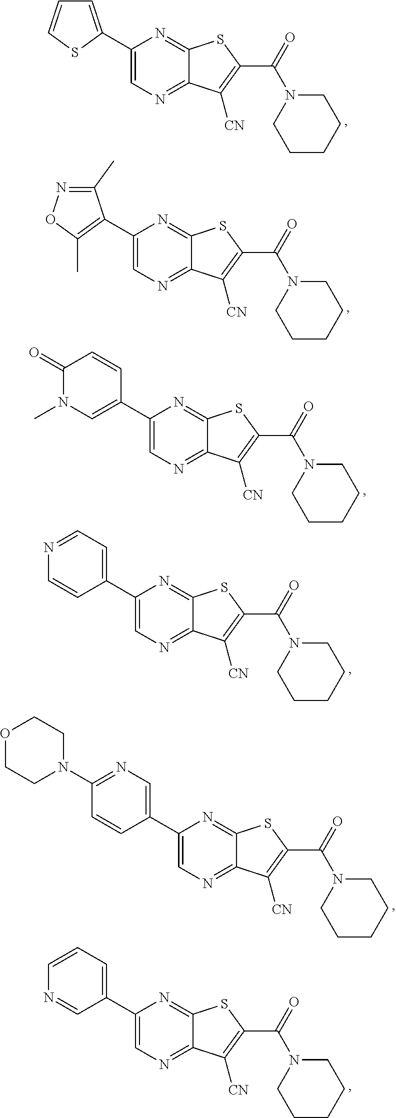 [SMILES: N#Cc1c(C(=O)N2CCCCC2)sc2nc(-c3cccs3)cnc12 ; Cc1noc(C)c1-c1cnc2c(C#N)c(C(=O)N3CCCCC3)sc2n1 ; Cn1cc(-c2cnc3c(C#N)c(C(=O)N4CCCCC4)sc3n2)ccc1=O ; N#Cc1c(C(=O)N2CCCCC2)sc2nc(-c3ccncc3)cnc12 ; N#Cc1c(C(=O)N2CCCCC2)sc2nc(-c3ccc(N4CCOCC4)nc3)cnc12 ; N#Cc1c(C(=O)N2CCCCC2)sc2nc(-c3cccnc3)cnc12]

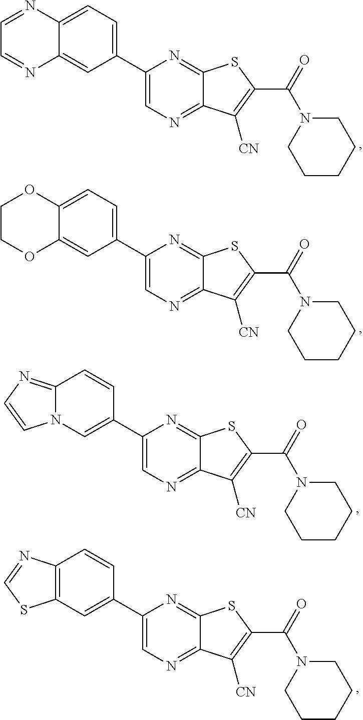 [SMILES: N#Cc1c(C(=O)N2CCCCC2)sc2nc(-c3ccc4nccnc4c3)cnc12 ; N#Cc1c(C(=O)N2CCCCC2)sc2nc(-c3ccc4c(c3)OCCO4)cnc12 ; N#Cc1c(C(=O)N2CCCCC2)sc2nc(-c3ccc4nccn4c3)cnc12 ; N#Cc1c(C(=O)N2CCCCC2)sc2nc(-c3ccc4ncsc4c3)cnc12]

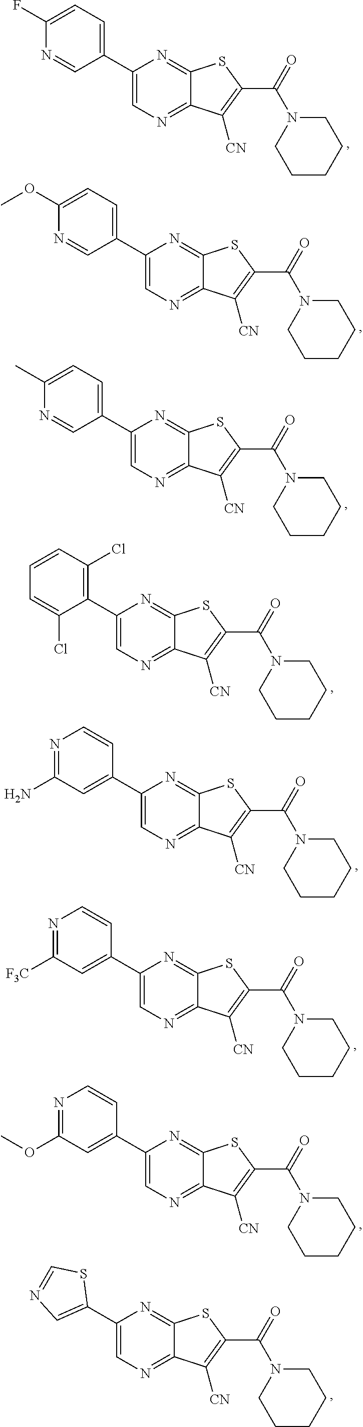

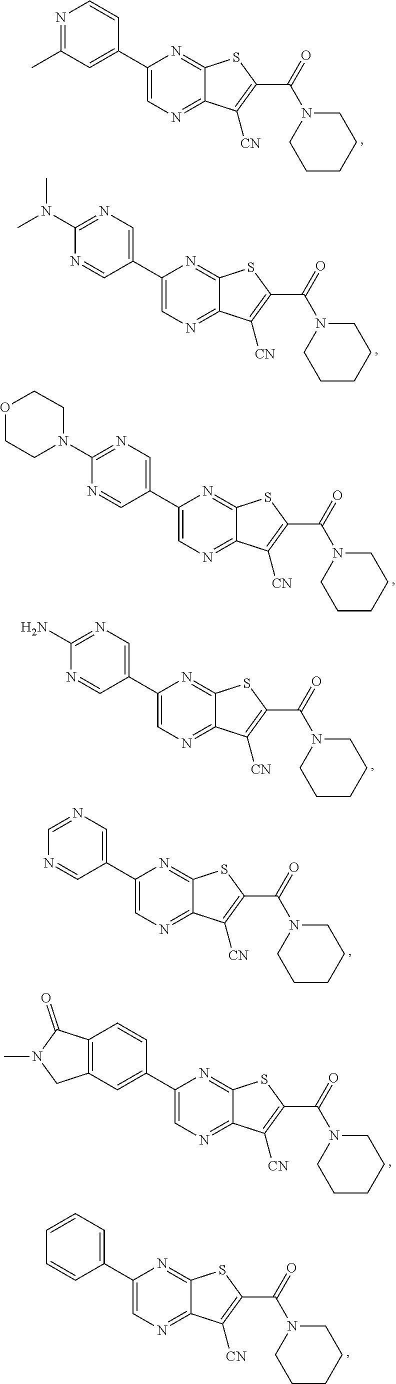

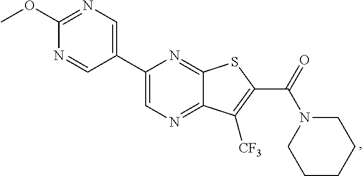 [SMILES: COc1ncc(-c2cnc3c(C(F)(F)F)c(C(=O)N4CCCCC4)sc3n2)cn1]

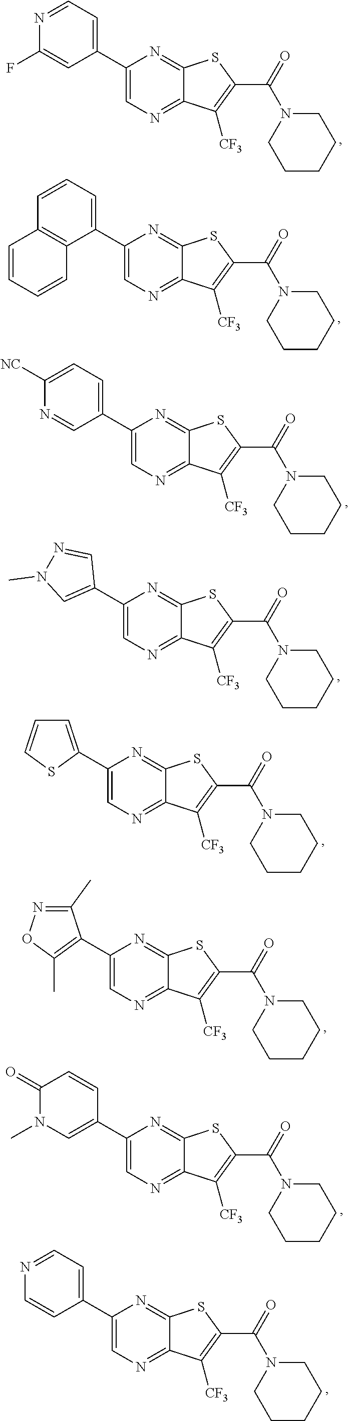

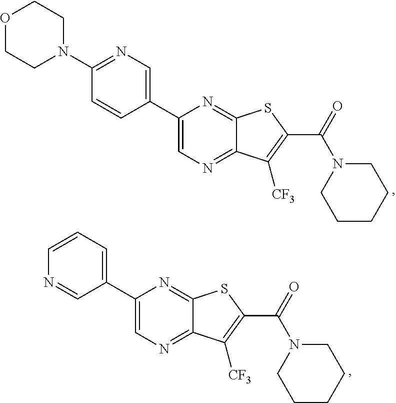 [SMILES: O=C(c1sc2nc(-c3ccc(N4CCOCC4)nc3)cnc2c1C(F)(F)F)N1CCCCC1 ; O=C(c1sc2nc(-c3cccnc3)cnc2c1C(F)(F)F)N1CCCCC1]

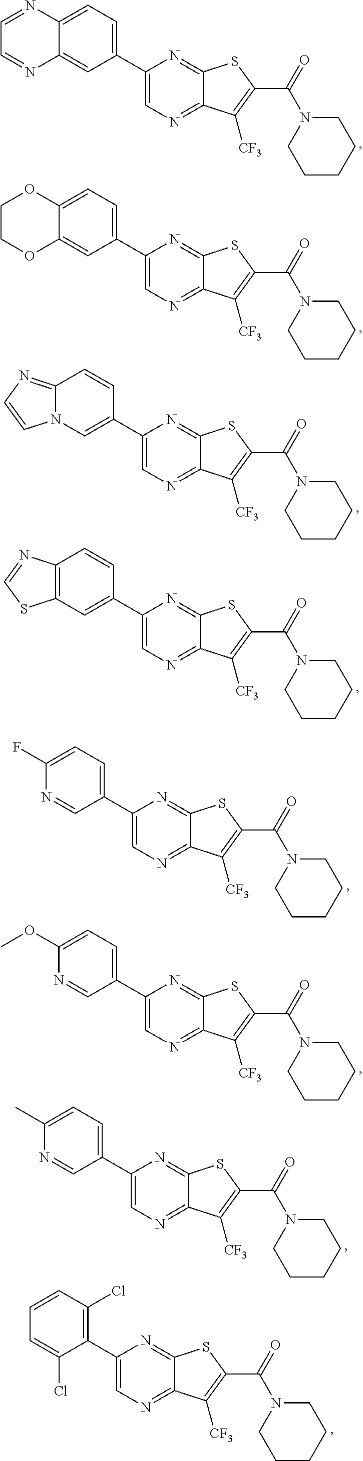

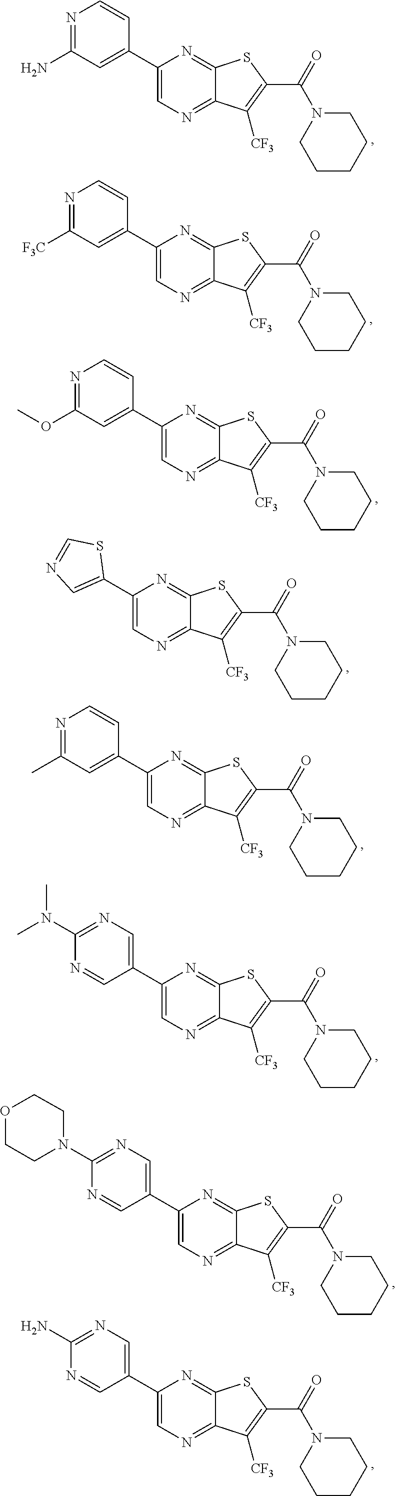

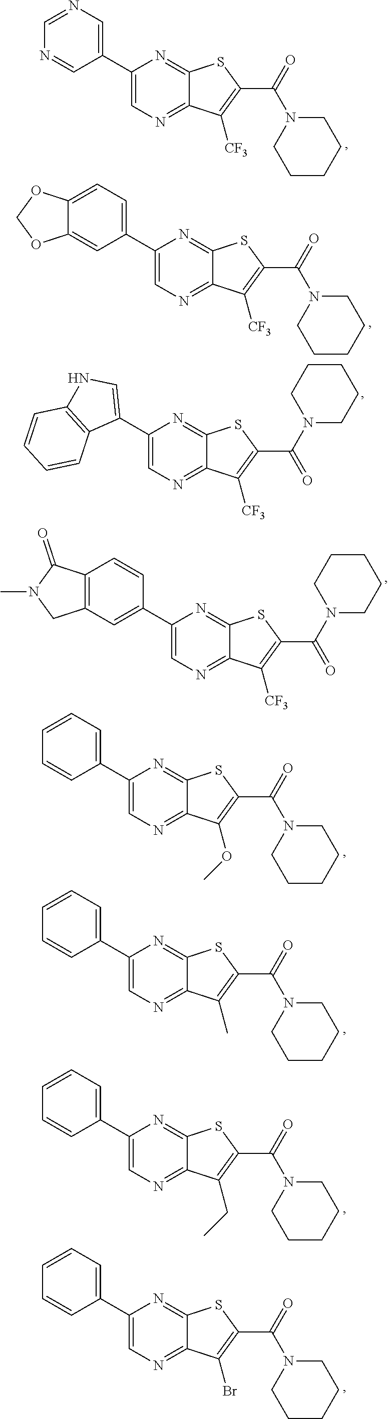

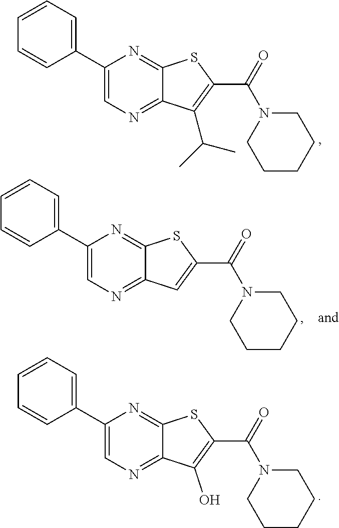 [SMILES: CC(C)c1c(C(=O)N2CCCCC2)sc2nc(-c3ccccc3)cnc12 ; O=C(c1cc2ncc(-c3ccccc3)nc2s1)N1CCCCC1 ; O=C(c1sc2nc(-c3ccccc3)cnc2c1O)N1CCCCC1]

32. A pharmaceutical composition, comprising at least one of the compound, the stereoisomer, tautomer or mixture form thereof, or the pharmaceutically acceptable salt thereof, or the solvate thereof, or the prodrug thereof according to claim 1, and at least one pharmaceutically acceptable excipient.

33. A method of treating or preventing a disease associated with 15-PGDH, comprising administering to a subject in need thereof the compound, the stereoisomer, tautomer or mixture form thereof, or the pharmaceutically acceptable salt thereof, or the solvate thereof, or the prodrug thereof according to claim 1, or a pharmaceutical composition thereof.

34. The method of according to claim 33, wherein the medicament is used for treating or preventing fibrosis, oral ulcer, gum disease, colitis, ulcerative colitis, gastroduodenal ulcer, inflammatory disease, vascular insufficiency, Raynaud's disease, Buerger's disease, neuropathy, pulmonary arterial hypertension, cardiovascular and renal disease, cardiovascular disease, trauma, skin damage, autoimmune disease, graft-versus-host disease, osteoporosis, ear disease, eye disease, neutropenia, diabetes mellitus, underactive bladder, or for promoting hair growth, pigmentation, tissue repair, tissue regeneration, implant in stem cell transplantation or bone marrow transplantation or organ transplantation, neurogenesis and neuronal cell death, or muscle regeneration, and cervical ripening, or for enhancing resistance to the toxicity of radiation exposure, the toxicity of chemotherapy and the toxicity of immunosuppressants.
